SEQUENCE LISTING (34 sequences)

SEQ ID NO: 1 - protein, 15 aa, synthetic construct
RASKGVSTSGYSYLH

SEQ ID NO: 2 - protein, 7 aa, synthetic construct
LASYLES

SEQ ID NO: 3 - protein, 9 aa, synthetic construct
QHSRDLPLT

SEQ ID NO: 4 - protein, 111 aa, synthetic construct [sequence omitted]

SEQ ID NO: 5 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, synthetic construct
NYYMY

SEQ ID NO: 7 - protein, 17 aa, synthetic construct
GINPSNGGTNFNEKFKN

SEQ ID NO: 8 - protein, 11 aa, synthetic construct
RDYRFDMGFDY

SEQ ID NO: 9 - protein, 120 aa, synthetic construct [sequence omitted]

SEQ ID NO: 10 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 11 aa, synthetic construct
RASQSVSSYLA

SEQ ID NO: 12 - protein, 7 aa, synthetic construct
DASNRAT

SEQ ID NO: 13 - protein, 9 aa, synthetic construct
QQSSNWPRT

SEQ ID NO: 14 - protein, 107 aa, synthetic construct [sequence omitted]

SEQ ID NO: 15 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, synthetic construct
NSGMH

SEQ ID NO: 17 - protein, 17 aa, synthetic construct
VIWYDGSKRYYADSVKG

SEQ ID NO: 18 - protein, 4 aa, synthetic construct
NDDY

SEQ ID NO: 19 - protein, 113 aa, synthetic construct [sequence omitted]

SEQ ID NO: 20 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 15 aa, synthetic construct
RASKSVSTSGFSYLH

SEQ ID NO: 22 - protein, 7 aa, synthetic construct
LASNLES

SEQ ID NO: 23 - protein, 9 aa, synthetic construct
QHSWELPLT

SEQ ID NO: 24 - protein, 5 aa, synthetic construct
SYYLY

SEQ ID NO: 25 - protein, 17 aa, synthetic construct
GVNPSNGGTNFSEKFKS

SEQ ID NO: 26 - protein, 11 aa, synthetic construct
RDSNYDGGFDY

SEQ ID NO: 27 - protein, 120 aa, synthetic construct [sequence omitted]

SEQ ID NO: 28 - protein, 111 aa, synthetic construct [sequence omitted]

SEQ ID NO: 29 - protein, 111 aa, synthetic construct
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 30 - protein, 111 aa, synthetic construct
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 31 - protein, 447 aa, synthetic construct; identical to SEQ ID NO: 10

SEQ ID NO: 32 - protein, 218 aa, synthetic construct; identical to SEQ ID NO: 5

SEQ ID NO: 33 - protein, 218 aa, synthetic construct
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 218 aa, synthetic construct
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0356947 A1
(43) Pub. Date: Nov. 20, 2025

(54) GENOME WIDE TUMOR DERIVED GENE EXPRESSION BASED SIGNATURES ASSOCIATED WITH POOR PROGNOSIS FOR MELANOMA PATIENTS WITH EARLY STAGE DISEASE

(71) Applicant: MERCK SHARP & DOHME LLC, Rahway, NJ (US)

(72) Inventors: Andrey LOBODA, Canton, MA (US); Michael NEBOZHYN, Colmar, PA (US)

(73) Assignee: MERCK SHARP & DOHME LLC, Rahway, NJ (US)

(21) Appl. No.: 18/874,709

(22) PCT Filed: Jun. 14, 2023
(86) PCT No.: PCT/US2023/025241

§ 371 Date: Dec. 13, 2024

Related U.S. Application Data
(60) Provisional application No. 63/353,074, filed on Jun. 17, 2022.

(51) Int. Cl. G16B 25/10; G16B 40/20

(52) U.S. Cl. CPC G16B 25/10; G16B 40/20

(57) ABSTRACT

The invention relates to a gene expression based biomarker that is predictive of patient clinical need for treatment that includes a PD-1 antagonist, wherein the gene expression based biomarker comprises five or more genes selected from the genes listed in Table 1 or Table 2 disclosed herein. More specifically, a negative level of a gene expression based biomarker wherein the biomarker comprises five or more genes selected from the genes listed in Table 1 or a positive level of a gene expression based biomarker wherein the biomarker comprises 5 or more genes selected from the genes listed in Table 2 is associated with favorable prognosis in a patient with cancer. Also provided are methods of treating a cancer patient with a PD-1 antagonist that were identified as positive for a gene expression based biomarker of the invention. The disclosure also provides methods and kits for testing tumor samples for the biomarkers.

### FIELD OF THE INVENTION

The invention relates generally to genomic prognostic genes and signatures for screening, diagnostics, and prognostics of cancer, which in some embodiments is melanoma. The invention relates to the utility of a gene signature in patient selection for future clinical trials. In addition, the invention relates to identifying patients who are likely to respond to or need further treatment with a PD-1 antagonist by determining if they are positive or negative for a gene expression based biomarker.

### REFERENCE TO SEQUENCE LISTING SUBMITTED ELECTRONICALLY

The sequence listing of the present application is submitted electronically via EFS-Web as an ASCII formatted sequence listing with a file name “25540WOPCT-SequenceListing”, with a creation date of May 24, 2023, and a size of 32.7 KB. This sequence listing submitted via EFS-Web is part of the specification and is herein incorporated by reference in its entirety.

### BACKGROUND OF THE INVENTION

Melanoma is a type of skin cancer that develops when melanocytes start to grow out of control. Melanoma accounts for only 1% of skin cancers but cause a large majority of skin cancer deaths (www.cancer.org/cancer/melanoma-skin-cancer/treating/immunotherapy). Melanoma is likely to spread to other parts of the body if early detection and treatment is not sought early.

Pembrolizumab, nivolumab, and ipilimumab block proteins that normally suppress the T-cell immune response against melanoma cells. Pembrolizumab and nivolumab are drugs that target PD-1, a protein on immune system cells called T cells that normally help keep these cells from attacking other cells in the body. By blocking PD-1, these drugs boost the immune response against melanoma cells.

Gene expression based biomarkers have been implemented successfully for tumor characterization, classification, and prediction of disease outcome. Gene expression based biomarkers have been described in the literature and are currently used to guide the use of therapy for melanoma in the market.

Prognostic factors are critical to distinguish patients with poor prognosis, likely to advance from primary melanoma to metastatic melanoma, and therefore, those that would benefit from further treatment. It is also critical to distinguish patients with favorable prognosis.

Previous research has explored relationships between biological gene expression signatures and pembrolizumab response. (Cristescu, R. et al., Transcriptomic Determinants of Response to Pembrolizumab Monotherapy across Solid Tumor Types, Clin Cancer Res, 28 (8): 1680-1689 (2022)).

PD-1 is recognized as an important player in immune regulation and the maintenance of peripheral tolerance. PD-1 is moderately expressed on naive T, B and NKT cells and up-regulated by TiB cell receptor signaling on lymphocytes, monocytes and myeloid cells (Sharpe et al., The function of programmed cell death 1 and its ligands in regulating autoimmunity and infection. Nature Immunology, 8:239-245 (2007)).

Two known ligands for PD-1, PD-L1 (B7-H1) and PD-L2 (B7-DC), are expressed in human cancers arising in various tissues. In large sample sets of e.g., ovarian, renal, colorectal, pancreatic, liver cancers and melanoma, it was shown that PD-L1 expression correlated with poor prognosis and reduced overall survival irrespective of subsequent treatment (Dong et al., Nat Med. 8(8):793-800 (2002); Yang et al. Invest Ophthalmol Vis Sci. 49: 2518-2525 (2008), Ghebeh et al. Neoplasia 8:190-198 (2006); Hamanishi et al., Proc. Natl. Acad. Sci. ISA 104: 3360-3365 (2007); Thompson et al., Cancer 5: 206-211 (2006): Nomi et al., Clin. Cancer Research 13:2151-2157 (2007); Ohigashi et al., Clin. Cancer Research 11: 2947-2953 (2005); Inman et al., Cancer 109: 1499-1505 (2007); Shimauchi et al. Int. J. Cancer 121:2585-2590 (2007); Gao et al. Clin. Cancer Research 15: 971-979 (2009); Nakanishi J. Cancer Immunol Immunother. 56: 1173-1182 (2007); and Hino et al., Cancer 00: 1-9 (2010)).

Similarly, PD-1 expression on tumor infiltrating lymphocytes was found to mark dysfunctional T cells in breast cancer and melanoma (Ghebeh et al, BMC Cancer. 8.5714-15 (2008); Ahmadzadeh et al., Blood 114 1537-1544 (2009)) and to correlate with poor prognosis in renal cancer (Thompson et al., Clinical Cancer Research 15: 1757-1761 (2007)). Thus, it has been proposed that PD-L1 expressing tumor cells interact with PD-1 expressing T cells to attenuate T cell activation and evasion of immune surveillance, thereby contributing to an impaired immune response against the tumor.

Immune checkpoint therapies targeting the PD-1 axis have resulted in groundbreaking improvements in clinical response in multiple human cancers (Brahmer et al., N Engl J Med 2012, 366: 2455-65; Garon et al. N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54, Topalian et al., J Clin Oncol 2014, 32: 1020-30; Wolchok et al., N Engl J Med 2013, 369: 122-33). Immune therapies targeting the PD-1 axis include monoclonal antibodies directed to the PD-1 receptor (KEYTRUDA™ (pembrolizumab), Merck Sharp & Dohme LLC, Rahway, NJ, USA; OPDIVOT™ (nivolumab), Bristol-Myers Squibb Company, Princeton, NJ, USA, and LIBTAYO™ (cemiplimab), Regeneron Pharmaceuticals, Inc., Tarrytown, NY, USA) and also those that bind to the PD-L1 ligand (MPDL3280A; TECENTRIQ™ (atezolizumab), Genentech, San Francisco, CA, USA; IMFINZI™ (durvalumab), AstraZeneca Pharmaceuticals LP, Wilmington, DE; BAVENCIO™ (avelumab), Merck KGaA, Darmstadt, Germany; JEMPERLM (dostarlimab), GlaxoSmithKline Biologics LLC, Philadelphia, PA, USA). Both therapeutic approaches have demonstrated anti-tumor effects in numerous cancer types.

Although PD-1 antagonists can induce durable anti-tumor responses in some patients in certain cancer types, a significant number of patients fail to respond to therapies targeting PD-1/PD-L1. Thus, a need exists for diagnostic tools to identify which cancer patients are most likely to achieve a clinical benefit to treatment with a PD-1 antagonist.

An active area in cancer research is the identification of intratumoral expression patterns for sets of genes, commonly referred to as gene signatures or molecular signatures, which are characteristic of particular types or subtypes of cancer, and which may be associated with clinical outcomes. PD-L1 immunohistochemistry and gene expression profiles (GEP) are associated with response to PD-1/PD-L1 inhibitor therapies in multiple tumor types (McDermott et al. Nat Med. 24:749-757 (2018); Ayers et al. J Clin Invest. 127:2930-2940 (2017); O'Donnell et al. J Clin Oncol. 35: 4502 (2017)). An 18-gene GEP was shown to be associated with a pan tumor response to pembrolizumab (Ayers et al., supra). A biomarker study of patients with cisplatin-ineligible advanced urothelial cancer who were enrolled in clinical trial Keynote-052 also showed that GEP was associated with response to pembrolizumab (O'Donnell et al., supra).

### SUMMARY OF THE INVENTION

The invention relates to the utility of a tumor derived gene expression profile associated with prognosis (e.g., likelihood of reoccurrence, metastatic disease progression, and poor overall survival) in patients with cancer. In particular, the invention relates to a gene expression based biomarker for identifying melanoma patients who are most likely to need treatment, e.g., treatment with a PD-1 antagonist.

Provided is a gene expression based biomarker for use in prognosing or classifying a patient who has been diagnosed with melanoma. The invention also relates to patient selection using a signature score derived from a gene expression based biomarker or comparison to a pre-specified threshold to identify patients who are most likely to need treatment. The invention further relates to predicting the survival or determining the prognosis of a patient and classifying them into a poor survival prognosis group or a favorable survival prognosis group based on signature score. Additionally, the invention relates to the identification of prognostic gene expression based biomarkers associated with differential expression between primary and metastatic disease.

Provided herein is a method for determining the prognosis of a melanoma patient comprising the steps: obtaining or receiving a sample from the tumor of a patient, determining the patient's biomarker expression profile, obtaining a biomarker reference expression profile associated with metastatic disease progression, determining the signature score from the biomarker expression profile, and classifying the patient with melanoma into a poor survival group or a favorable survival group, wherein the patient is classified into a poor survival prognosis group if the tumor is classified as biomarker positive, and wherein the patient with poor survival prognosis can be further treated as applicable.

Also provided herein is a method for testing a tumor for the presence or absence of a biomarker that predicts poor prognosis in early stage disease, thereby allowing early treatment, which comprises, (a) obtaining a sample from the tumor, (b) measuring the raw RNA expression level in the tumor sample for each gene in a gene signature, (c) performing necessary normalization, and (d) calculating the arithmetic mean of the normalized RNA expression levels of the genes in the signature to generate a score for the gene expression based biomarker; wherein the gene expression based biomarker comprises at least 5 genes selected from the group consisting of the genes listed in Table 1 or at least 5 genes selected from the group consisting of the genes listed in Table 2, or at least 5 genes selected from the group consisting of the genes listed in Table 1 and Table 2, (e) comparing the calculated score to a reference score for the gene expression based biomarker; and (f) classifying the tumor as biomarker positive or biomarker negative; wherein if the calculated score is equal to or greater than the reference score or pre-specified threshold, then the tumor is classified as biomarker positive, and if the calculated gene expression based biomarker signature score is less than the reference score or pre-specified threshold, then the tumor is classified as biomarker negative, and wherein the patient is determined to have a poor prognosis if the tumor is classified as biomarker positive and a favorable prognosis if the tumor is classified as biomarker negative. The patient is determined to have a poor prognosis if the tumor is classified as biomarker positive for a gene expression based biomarker defined by 5 or more genes from Table 1 and a favorable prognosis if the tumor is classified as biomarker negative for a gene expression based biomarker defined by 5 or more genes from Table 1. The patient is determined to have a poor prognosis if the tumor is classified as biomarker positive for a gene expression based biomarker defined by 5 or more genes from Table 2 and a favorable prognosis if the tumor is classified as biomarker negative for a gene expression based biomarker defined by 5 or more genes from Table 2. In additional aspects, the invention relates to a method of treatment of a patient who is determined to have a poor prognosis using the methods defined herein, wherein the patient is treated with a PD-1 antagonist.

The invention further relates to a method for treating cancer in a patient having a tumor which comprises administering to the patient a PD-1 antagonist if the tumor is positive for a gene expression based biomarker defined by 5 or more genes from Table 1, or administering to the patient a cancer treatment that does not include a PD-1 antagonist if the tumor is negative for the biomarker.

The invention further relates to a method of treating cancer in a patient having a tumor which comprises administering to the patient a PD-1 antagonist if the tumor is positive for a gene expression based biomarker defined by 5 or more genes from Table 2, or administering to the patient a cancer treatment that does not include PD-1 antagonist if the tumor is negative for the biomarker.

### DESCRIPTION OF THE DRAWINGS

FIGS. 1A, 1B, and 1C are volcano plots showing statistically significant values (p-values, adjusted for false discovery rate) versus magnitude of change (receiver operating characteristic area under the curve (ROC AUC)) for all genes screened, across all three evaluated data sets (Merck-Moffitt dataset, TCGA dataset, and M2GEN dataset). See Example 1.

FIGS. 2A, 2B, and 2C are histograms and overlaid cumulative distribution plots that show a comparison of the distribution ROC AUC for metastatic versus primary tumors across all genes screened within the three data sets. See Example 1D.

FIGS. 3A, 3B, 3C, 3D, 3F, and 3F are histograms and overlaid cumulative distribution plots that show the distribution of all pairwise correlations between genes in sets identified in Merck-Moffitt melanoma data sets to be differentially expressed between metastatic and primary tumors, within three data sets.

FIGS. 4A and 4B are scatterplots that show ROC AUC for metastatic versus primary tumors differential expression comparing results obtained in Merck-Moffitt data set to the TCGA and M2GEN melanoma tumor data sets.

FIGS. 5A, 5B, and 5C are scatterplots between signature scores based on the average expression of genes in signature-up and signature-down selected by differential expression in metastatic versus primary tumors in Merck Moffitt melanomas. FIGS. 12A, 12B and 12C show consistent and significant anti-correlation of signature-up and signature-down scores observed in expression data in three sets (Merck-Moffitt, TCGA, and M2GEN are shown in 5A, 5B, and 5C respectively).

FIGS. 6A, 6B, and 6C are ROC AUC curves illustrating the association between proposed gene expression signature score and metastatic versus primary status in each individual set (Merck-Moffitt, TCGA, and M2GEN in 6A, 6B, and 6C respectively).

FIGS. 7A, 7B, and 7C are superimposed violin and boxplots illustrating distributions of proposed gene expression signature scores within and between primary and metastatic melanoma tumors in each data set (Merck-Moffitt, TCGA, and M2GEN are shown in 7A, 7B, and 7C respectively).

FIGS. 8A, 8B, and 8C are sorted waterfall plots illustrating distributions and differences in distributions of proposed gene expression signature scores between metastatic and primary melanoma tumors (Merck-Moffitt, TCGA, and M2GEN are shown in 8A, 8B, and 8C respectively).

FIGS. 9A, 9B, and 9C are two-dimensional heat map plots showing correlations among metastatic versus primary status, proposed de novo signature scores, and additional gene expression signatures (Merck-Moffitt, TCGA, and M2GEN are shown in 9A, 9B, and 9C respectively).

FIGS. 10A, 10B, and 10C are scatterplots showing primary signature score compared to stromal/EMT/TGFb consensus signature score in metastatic versus primary melanoma in the three data sets (Merck-Moffitt, TCGA, and M2GEN are shown in 10A, 10B, and 10C respectively).

### DETAILED DESCRIPTION OF THE INVENTION

The invention relates to a gene expression based biomarker that is predictive of a patient's prognosis, wherein the patient has melanoma. More specifically, the invention relates to a gene expression based biomarker that is predictive of a patient's need to be treated, for example, treatment with a PD-1 antagonist.

### I. Definitions and Abbreviations

Throughout the detailed description and examples of the invention the following abbreviations will be used:
BOR best overall response
CDR complementarity determining region
CHO Chinese hamster ovary
CPS combined positive score
CR complete response
DFS disease free survival
ECOG Eastern Cooperative Oncology Group
EMT epithelial to mesenchymal transition
FFPE formalin-fixed, paraffin-embedded
FR framework region
GEP gene expression profile
IHC immunohistochemistry or immunohistochemical
irRC immune related response criteria
NCBI National Center for Biotechnology Information
NPV net predictive value
NR not reached
OR overall response
OS overall survival
PD progressive disease
PD-1 programmed death 1
PD-L1 programmed cell death 1 ligand 1
PD-L2 programmed cell death 1 ligand 2
PFS progression free survival
PPV positive predictive value
PR partial response
Q2W one dose every two weeks
Q3W one dose every three weeks
Q4W one dose every four weeks
Q6W one dose every six weeks
RECIST Response Evaluation Criteria in Solid Tumors
ROC receiver operating characteristic
SD stable disease
TGFβ transforming growth factor-β
UC urothelial cancer
VH immunoglobulin heavy chain variable region
VK immunoglobulin kappa light chain variable region

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“About” when used to modify a numerically defined parameter (e.g., the gene signature score for a gene signature discussed herein, or the dosage of a PD-1 antagonist, or the length of treatment time with a PD-1 antagonist, or the amount of time between treatments with a PD-1 antagonist) means that the parameter may vary by as much as 10% above or below the stated numerical value for that parameter. For example, a gene signature consisting of about 10 genes may have between 9 and 11 genes. Similarly, a reference gene signature score of about 2.462 includes scores of and any score between 2.2158 and 2.708. In certain embodiments, “about” can mean a variation of ±0.1%, ±0.5%, ±1%, ±2%, ±3%, ±4%, ±5%, ±6%, ±7%, ±8%, ±9% or ±10%. When referring to the amount of time between administrations in a therapeutic treatment regimen (i.e., amount of time between administrations of the PD-1 antagonist, e.g., “about 6 weeks,” which is used interchangeably herein with “approximately every six weeks”), “about” refers to the stated time t a variation that can occur due to patient/clinician scheduling and availability around the 6-week target date. For example, “about 6 weeks” can refer to 6 weeks ±5 days, 6 weeks ±4 days, 6 weeks ±3 days, 6 weeks ±2 days or 6 weeks ±1 day, or may refer to 5 weeks, 2 days through 6 weeks, 5 days.

“Administration” and “treatment,” as it applies to an animal, human, experimental subject, patient, cell, tissue, organ, or biological fluid, refers to contact of an exogenous pharmaceutical, therapeutic, diagnostic agent, or composition to the animal, human, subject, cell, tissue, organ, or biological fluid.

“Treat” or “treating” a cancer, as used herein, means to administer a PD-1 antagonist, e.g., an anti-PD-1 antibody or antigen binding fragment thereof, to a patient having a cancer, or diagnosed with a cancer, to achieve at least one positive therapeutic effect, such as, reduced number of cancer cells, reduced tumor size, reduced rate of cancer cell infiltration into peripheral organs, or reduced rate of tumor metastasis or tumor growth. “Treatment” may include one or more of the following: inducing/increasing an antitumor immune response, decreasing the number of one or more tumor markers, halting or delaying the growth of a tumor or blood cancer or progression of disease associated with PD-1 binding to its ligands PD-L1 and/or PD-L2 (“PD-1-related disease”) such as cancer, stabilization of PD-1-related disease, inhibiting the growth or survival of tumor cells, eliminating or reducing the size of one or more cancerous lesions or tumors, decreasing the level of one or more tumor markers, ameliorating or abrogating the clinical manifestations of PD-1-related disease, reducing the severity or duration of the clinical symptoms of PD-1-related disease such as cancer, prolonging the survival of a patient relative to the expected survival in a similar untreated patient, and inducing complete or partial remission of a cancerous condition or other PD-1 related disease.

Positive therapeutic effects in cancer can be measured in a number of ways (See, W. A. Weber, J. Nucl. Med. 50:1S-10S (2009)). In some embodiments, response to a PD-1 antagonist is assessed using RECIST 1.1 criteria or irRC. With respect to tumor growth inhibition, according to NCI standards, a tumor volume over control volume (TIC)≤542% is the minimum level of anti-tumor activity. A T/C<10% is considered a high anti-tumor activity level, with T/C (%)=Median tumor volume of the treated/Median tumor volume of the control×100. In some embodiments, the treatment achieved by a therapeutically effective amount is any of progression free survival (PFS), disease free survival (DFS) or overall survival (OS). In some embodiments, the treatment achieved by a therapeutically effective amount is any of partial response (PR), complete response (CR), PFS, DFS, overall response (OR) or OS.

PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the treatment methods, compositions and uses of the present invention may not be effective in achieving a positive therapeutic effect in every patient, it should do so in a statistically significant number of patients as determined by any statistical test known in the art such as the Student's t-test, the chi²-test, the U-test according to Mann and Whitney, the Kruskal-Wallis test (H-test), Jonckheere-Terpstra-test and the Wilcoxon-test.

In some embodiments, a gene signature biomarker of the invention predicts whether a patient with a solid tumor is likely to achieve a PR or a CR. The dosage regimen of a therapy described herein that is effective to treat a cancer patient may vary according to factors such as the disease state, age, and weight of the patient, and the ability of the therapy to elicit an anti-cancer response in the patient.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of a parental antibody generated in a mouse for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxyl-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chain variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia et al., (1987) J Mol. Biol. 196 901-917 or Chothia et al., (1989) Nature 342-878-883.

As used herein, the term “hypervariable region” refers to the amino acid residues of an antibody that are responsible for antigen-binding. The hypervariable region comprises amino acid residues from a “complementarity determining region” or “CDR” (i.e. CDRL1, CDRL2 and CDRL3 in the light chain variable domain and CDRH1, CDRH2 and CDRH3 in the heavy chain variable domain). See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Riol 196: 901-917 (defining the CDR regions of an antibody by structure). As used herein, the term “framework” or “FR” residues refers to those variable domain residues other than the hypervariable region residues defined herein as CDR residues.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g., fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g., the amino acid sequence of a mature human PD-1 or human PD-L1 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The humanized forms of rodent antibodies will generally comprise the same CDR sequences of the parental rodent antibodies, although certain amino acid substitutions may be included to increase affinity, increase stability of the humanized antibody, or for other reasons.

“Anti-tumor response” when referring to a cancer patient treated with a therapeutic agent, such as a PD-1 antagonist, means at least one positive therapeutic effect, such as for example, reduced number of cancer cells, reduced tumor size, reduced rate of cancer cell infiltration into peripheral organs, reduced rate of tumor metastasis or tumor growth, or progression free survival. Positive therapeutic effects in cancer can be measured in a number of ways (See, W. A. Weber, J. Null. Med. 50:1S-10S (2009); Eisenhauer et al., supra). In some embodiments, an anti-tumor response to a PD-1 antagonist is assessed using RECIST 1.1 criteria, bidimensional irRC or unidimensional irRC. In some embodiments, an anti-tumor response is any of SD, PR, CR, PFS, DFS. In some embodiments, a gene signature biomarker of the invention predicts whether a patient with a solid tumor is likely to achieve a PR or a CR.

“Bidimensional irRC” refers to the set of criteria described in Wolchok J D, et al. Guidelines for the evaluation of immune therapy activity in solid tumors: immune-related response criteria. Clin Cancer Res. 2009, 15(23):7412-7420. These criteria utilize bidimensional tumor measurements of target lesions, which are obtained by multiplying the longest diameter and the longest perpendicular diameter (cm²) of each lesion.

“Biotherapeutic agent” means a biological molecule, such as an antibody or fusion protein, that blocks ligand/receptor signaling in any biological pathway that supports tumor maintenance and/or growth or suppresses the anti-tumor immune response.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin lymphoma, non-Hodgkin lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Particularly preferred cancers that may be treated in accordance with the present invention include those characterized by elevated expression of one or both of PD-L1 and PD-L2 in tested tissue samples.

“CDR” or “CDRs” as used herein means complementarity determining region(s) in an immunoglobulin variable region, generally defined using the Kabat numbering system.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytotoxic/antitumor antibiotics, topoisomerase inhibitors, photosensitizers, anti-estrogens and selective estrogen receptor modulators (SERMs), anti-progesterones, estrogen receptor down-regulators (ERDs), estrogen receptor antagonists, luteinizing hormone-releasing hormone agonists, anti-androgens, aromatase inhibitors, EGFR inhibitors, VEGF inhibitors, anti-sense oligonucleotides that that inhibit expression of genes implicated in abnormal cell proliferation or tumor growth. Chemotherapeutic agents useful in the treatment methods of the present invention include cytostatic and/or cytotoxic agents.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, if a gene signature score is defined as the composite RNA expression score for a set of genes that consists of a specified list of genes, the skilled artisan will understand that this gene signature score could include the RNA level determined for one or more additional genes, preferably no more than three additional genes, if such inclusion does not materially affect the predictive power.

“Framework region” or “FR” as used herein means the immunoglobulin variable regions excluding the CDR regions.

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared ×100. For example, if 8 of 10 of the positions in two sequences are matched or homologous when the sequences are optimally aligned then the two sequences are 80% homologous Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990)J. Mol. Biol. 215:403-410; Gish, W., et al., (1993). Nature Genet. 3:266-272; Madden, T. L., et al., (1996)Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J M. et al., (1994) Comput. Appl. Biosci. 10:67-70; ALIGNMENT SCORING SYSTEMS: Dayhoff, M. O., et al., “A model of evolutionary change in proteins “in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et a. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Oligonucleotide” refers to a nucleic acid that is usually between 5 and 100 contiguous bases in length, and most frequently between 10-50, 10-40, 10-30, 10-25, 10-20, 15-50, 15-40, 15-30, 15-25, 15-20, 20-50, 20-40, 20-30 or 20-25 contiguous bases in length.

The term “patient” (alternatively referred to as “subject” or “individual” herein) refers to a mammal (e.g., rat, mouse, dog, cat, rabbit) capable of being treated with the methods and compositions of the invention, most preferably a human, or to a cell sample, tissue sample or organ sample derived therefrom, including, for example, cultured cell lines, a biopsy, a blood sample, or a fluid sample containing a cell or a plurality of cells. In some embodiments, the patient is an adult patient. In other embodiments, the patient is a pediatric patient.

“PD-1 antagonist” means any chemical compound or biological molecule that blocks binding of PD-L1 to PD-1 and preferably also blocks binding of PD-L2 to PD-1. As a none limiting example, a PD-1 antagonist blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the various aspects and embodiments of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists useful in the any of the various aspects and embodiments of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments, the human constant region is selected from the group consisting of IgG1, IgG2, IgG3 and IgG4 constant regions, and in some embodiments, the human constant region is an IgG1 or IgG4 constant region. In some embodiments, the antigen binding fragment is selected from the group consisting of Fab, Fab′-SH, F(ab′)₂, scFv and Fv fragments.

Examples of mAbs that bind to human PD-1, and useful in the various aspects and embodiments of the present invention, are described in U.S. Pat. Nos. 7,521,051, 8,008,449, and 8,354,509. Specific anti-human PD-1 mAbs useful as the PD-1 antagonist various aspects and embodiments of the present invention include: pembrolizumab, a humanized IgG4 mAb with the structure described in WHO Drug Information, Vol. 27, No. 2, pages 161-162 (2013), nivolumab (BMS-936558), a human IgG4 mAb with the structure described in WHO Drug Information, Vol. 27, No. 1, pages 68-69 (2013); pidilizumab (CT-011, also known as hBAT or hBAT-1); and the humanized antibodies h409A11, h409A16 and h409A17, which are described in WO 2008/156712.

Additional PD-1 antagonists useful in any of the various aspects and embodiments of the present invention include a pembrolizumab biosimilar or a pembrolizumab variant.

As used herein “pembrolizumab biosimilar” means a biological product that (a) is marketed by an entity other than Merck and Co., Inc. (Rahway, N J., USA), or a subsidiary thereof, and (b) is approved by a regulatory agency in any country for marketing as a pembrolizumab biosimilar. In an embodiment, a pembrolizumab biosimilar comprises a pembrolizumab variant as the drug substance. In an embodiment, a pembrolizumab biosimilar has the same amino acid sequence as pembrolizumab.

As used herein, a “pembrolizumab variant” means a monoclonal antibody which comprises heavy chain and light chain sequences that are identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

Examples of mAbs that bind to human PD-L1, and useful in any of the various aspects and embodiments of the present invention, are described in WO2013/019906, WO2010/077634 and U.S. Pat. No. 8,383,796. Specific anti-human PD-L1 mAbs useful as the PD-1 antagonist in the various aspects and embodiments of the present invention include atezolizumab, BMS-936559, MEDI4736, avelumab and durvalumab.

Other PD-1 antagonists useful in any of the various aspects and embodiments of the present invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesin on molecules that specifically bind to PD-1 are described in WO 2010/027827 and WO 2011/066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

“Probe” as used herein means an oligonucleotide that is capable of specifically hybridizing under stringent hybridization conditions to a transcript expressed by a gene of interest.

“RECIST 1.1 Response Criteria” as used herein means the definitions set forth in Eisenhauer et al., E. A. et al., Eur. J Cancer 45:228-247 (2009) for target lesions or non-target lesions, as appropriate based on the context in which response is being measured.

“Gene expression based biomarker signature score” as used herein means the score for a gene expression based biomarker that has been determined to divide at least the majority of responders from at least the majority of non-responders in a reference population of patients who have the same tumor type as a test patient and may have been treated with a PD-1 antagonist or who will be evaluated for treatment with a PD-1 antagonist. Preferably, at least any of 60%, 70%, 80%, or 90% of responders in the reference population will have a gene expression based biomarker signature score that is above the selected reference score, while the gene expression based biomarker signature score for at least any of 60%/u, 70% 80%, 90/u or 95% of the non-responders in the reference population will be lower than the selected reference score. In some embodiments, the negative predictive value of the reference score is greater than the positive predictive value. In some embodiments, responders in the reference population are defined as patients who achieved a partial response (PR) or complete response (CR) as measured by RECIST 1.1 criteria and non-responders are defined as not achieving any RECIST 1.1 clinical response. In other embodiments, patients in the reference population are treated with substantially the same anti-PD-1 therapy as that being considered for the test patient, i.e., administration of the same PD-1 antagonist using the same or a substantially similar dosage regimen.

“Sample” when referring to a tumor or any other biological material referenced herein, means a tissue sample that has been removed from the patient's tumor; thus, the testing methods described herein are not performed in or on the patient (although the methods of treatment of the invention clearly include treating the patient).

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Tissue section” refers to a single part or piece of a tissue sample, e.g., a thin slice of tissue cut from a sample of a normal tissue or of a tumor.

“Tumor” as it applies to a patient diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g., by measuring the dimensions of tumor(s) upon removal from the patient, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g., by measuring the dimensions of tumor(s) upon removal from the patient, e.g., using calipers, or while in the body using imaging techniques, e.g., bone scan, ultrasound, CT or MRI scans.

“Unidimensional irRC” refers to the set of criteria described in Nishino M, Giobbie-Hurder A, Gargano M, Suda M, Ramaiya N H, and Hodi F S., Developing a Common Language for Tumor Response to Immunotherapy: Immune-related Response Criteria using Unidimensional measurements. Clin Cancer Res. 2013; 19(14):3936-3943 These criteria utilize the longest diameter (cm) of each lesion.

“Variable regions” or “V region” as used herein means the segment of IgG chains which is variable in sequence between different antibodies. It extends to Kabat residue 109 in the light chain and 113 in the heavy chain.

As used herein, the term “favorable prognosis” in the context of melanoma means that a patient is not expected to further progress from primary melanoma to malignant melanoma and have no distant metastases of a melanoma tumor within five years of initial diagnosis of melanoma. Favorable prognosis allows patients to avoid any unnecessary further treatment. Those with favorable prognosis do not have an unmet medical need and have a good prognosis. Further, those with favorable prognosis are less likely to progress from melanoma with a primary tumor to metastatic melanoma.

As used herein, the term “poor prognosis” in the context of melanoma means that a patient is expected to progress from primary melanoma to malignant or metastatic melanoma within five years of initial diagnosis of melanoma. Further, those with poor prognosis are more likely to progress from primary melanoma to metastatic melanoma.

As used herein, the term “gene” has its meaning as understood in the art. However, it will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs and microRNAs. For clarity, the term “gene” generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid. In some cases, the gene includes regulatory sequences involved in transcription, or message production or composition. In other embodiments, the gene comprises transcribed sequences that encode for a protein, polypeptide, or peptide. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. In particular embodiments, the transcribed nucleotide sequence comprises at least one functional protein, polypeptide and/or peptide encoding unit. As will be understood by those in the art, this functional term “gene” includes both genomic sequences, RNA or cDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express, using nucleic acid manipulation technology, proteins, polypeptides, domains, peptides, fusion proteins, mutants and/or such like. The sequences which are located 5′ of the coding region and which are present on the mRNA are referred to as 5′ untranslated sequences (“5′UTR”). The sequences which are located 3′ or downstream of the coding region and which are present on the mRNA are referred to as 3′ untranslated sequences, or (“3′UTR”).

As used herein, the term “signature” or “gene signature” refers to a set of one or more differentially expressed genes that are statistically significant and characteristic of the biological differences between two or more cell samples, e.g., normal and diseased cells, cell samples from different cell types or tissue, or cells exposed to an agent or not. A signature may be expressed as a number of individual unique probes complementary to signature genes whose expression is detected when a cRNA product is used in microarray analysis or in a PCR reaction. A signature may be exemplified by a particular set of markers or a gene expression based biomarker.

“Primary melanoma” (or “early melanoma”) as used herein means the original tumor and/or refers to stage 0 or stage I melanoma. Stage 0 refers to melanoma in situ, which means melanoma cells are found only in the outer layer of skin or epidermis. Stage I refers to primary melanoma that is only in the skin and is relatively thin and divided into two groups depending on the thickness of the melanoma.

“Intermediate” or “high-risk melanoma,” also considered stage II melanoma, refers to melanoma that is thicker than stage I melanoma, extending through the epidermis and further into the dermis, the dense inner layer of skin. Stage II melanoma has a higher chance of spreading at this stage than primary melanoma.

“Advanced melanoma”, “malignant melanoma” or “metastatic melanoma” comprises stages III and IV which includes melanoma that has spread locally or through the lymphatic system to a regional lymph node. Stage IV describes melanoma that has spread through the bloodstream to other parts of the body. “Metastatic tumor,” as used herein, means a new tumor when the cancerous cells from the original tumor (primary tumor) get loose, spread through the lymph or blood circulation, and start a new tumor (metastatic tumor).

“Favorable survival” or “favorable prognosis,” as used herein, refers to an increased chance of survival as compared to patients in a “poor survival” group. For example, the biomarkers of the application can prognose or classify patients into a favorable survival group.

“Poor survival” or “poor prognosis,” as used herein, refers to an increased risk of death as compared to patients in a favorable survival group. For example, the biomarkers of the application can prognose or classify patients into a poor survival group.

### II. Gene Signatures and Utility of Gene Signature and Biomarkers of the Invention Up-Regulated Genes (Poor Prognosis Genes) and Down-Regulated Genes (Favorable Prognosis Genes)

In one embodiment, the invention identifies a genome wide tumor derived gene expression based biomarker that is associated with poor prognosis for patients suffering from melanoma. In a further embodiment, the invention provides a set of 128 genes whose expression is correlated with identifying a patient with poor prognosis for treating early stage melanoma. In yet a further embodiment, the invention comprises a gene expression based biomarker comprising up-regulated genes, wherein the gene expression based biomarker comprises 5 or more genes listed in Table 1 In a sub-embodiment, the invention provides the identification of a gene expression based biomarker that allows classification of a patient into a prognosis group, wherein the prognosis group is predictive of a patient's need of further treatment. In another sub-embodiment, the invention relates to the identification of a genome-wide tumor derived gene expression based biomarker that can be used in identifying, classifying, and/or selecting for melanoma patients with early disease (Stage 0 or Stage I), who may be in need of treatment.

In one embodiment, the invention provides a gene expression based biomarker comprising at least 5 genes listed in Table 1 that is correlated with a need of treatment for a patient who has been diagnosed with melanoma. In one embodiment, a patient is identified as a patient with poor prognosis if the patient has a higher expression of 5 or more poor prognosis genes listed in Table 1 (e.g., 5 ore more, 6 or more, 7 or more, 8 or more, 9 or more, 10 or more . . . 95 or more, 96 or more, 97 or more, 98, 99, or 100 genes from Table 1). In one embodiment, a patient is identified as a patient with good prognosis if the patient has a lower expression of 5 or more poor prognosis or up-regulated genes listed in Table 1.

In one embodiment, the invention provides a method of using a gene expression based biomarker to identify melanoma patients with a poor prognosis in early stage disease.

In one embodiment, the invention provides a method of treating a melanoma patient with early stage disease by identification of the patient with a gene expression based biomarker as described herein. In another embodiment, the invention provides a method of identifying melanoma patients who have metastatic melanoma versus primary melanoma. In yet a further embodiment, the invention relates to identification of a patient with a positive or elevated level of a gene expression based biomarker, wherein the gene expression based biomarker comprises 5 or more up-regulated genes (or poor prognosis genes) from Table 1, and wherein the patient with a positive or elevated level of a gene expression based biomarker based on up-regulated expression of genes is deemed to have a poor prognosis, and in need of further treatment options. A patient is positive for the gene expression based biomarker if the patient has a higher expression of up-regulated genes found in Table 1, or if a patient has a signature score about a pre-specified threshold. As a result, the tumor is classified as biomarker positive.

In some embodiments, the invention relates to identifying a melanoma patient having a poor prognosis. In a sub-embodiment, the patient having a poor prognosis is likely to have a reoccurrence of melanoma, metastatic disease progression, or poor overall survival.

In some embodiments of the invention, the melanoma is early stage. In one embodiment, the melanoma is primary melanoma. In another embodiment, the melanoma is metastatic melanoma.

In some embodiments, the invention relates to classifying a patient as having a poor prognosis based on a gene expression level by calculating an elevated level of a gene expression of 5 or more up-regulated genes listed in Table 1. A further sub-embodiment comprises classifying a patient as having a poor prognosis based on a gene expression level by calculating an elevated level of a gene expression of 5 or more up-regulated genes listed in Table 1, wherein an elevated gene expression level indicates a patient with a pathology related to metastatic melanoma, and wherein the patient is in need of further medical treatment.

In a sub-embodiment, the invention relates to classifying a patient as having a poor prognosis based on a gene expression level by calculating elevated level of a gene expression of 5 or more up-regulated genes listed in Table 1. Additionally, the invention relates to the calculation of elevated level of gene expression used in determining a threshold for patients in a clinical trial setting. A further sub-embodiment comprises changing the threshold dependent on clinical outcomes designated for the clinical trial.

In one embodiment, the invention relates to selecting those melanoma patients having a poor prognosis based on having an elevated expression level of 5 or more poor prognosis genes listed in Table 1 for participation in clinical trials to evaluate a patient's need for additional treatment and to facilitate efficacious treatments and therapies for patients with an unmet clinical need. The invention further relates to selecting those patients having a poor prognosis for clinical trials in order to effectively evaluate a new treatment method.

In one embodiment, the invention relates to classifying a patient having a favorable prognosis based on a gene expression level by calculating a decreased level of gene expression of 5 or more up-regulated genes listed in Table 1.

In one embodiment, the invention relates to identifying a gene expression based biomarker within a sample obtained from a patient to calculate a gene signature score.

In a further embodiment, the invention relates to calculating a gene signature score based on the up-regulated genes to determine a prognosis for a melanoma patient. In a further aspect, the classification of a prognosis for a melanoma patient allows for treatment with an appropriate treatment option. A patient having a favorable prognosis may not have a clinical need for additional treatment and can avoid possible side effects.

In one embodiment, the invention relates to the use of a gene expression based biomarker signature score for a gene expression based biomarker which comprises a set of at least about 5 of the up-regulated genes listed in Table 1 to determine prognosis of a melanoma patient.

In particular embodiments, the gene expression based biomarker comprises at least 5 (five) genes selected from the genes listed in Table 1. In other embodiments, the gene expression based biomarker comprises at least 5 genes, at least 6 genes, at least 7 genes, at least 8 genes, at least 9 genes, at least 10 genes, at least 11 genes, at least 12 genes, at least 13 genes, etc. or at least 128 genes from the genes listed in Table 1.

In one embodiment, the gene expression based biomarker comprises the following genes: ABHD10, ABHD3, ACVR2B, ADAL, ALG13, ANGEL1, ATG16L1, B4GALT3, BRAF, BRSK1, C12orf60, C1orf56, C4A, C7, CCDC151, CCDC93, CCNE1, CD1D, CD38, CD5L, CDC42SE1, CHEK2, CHORDC1, CMTM7, CPOX, CR1, CRELD1, CRNKL1, CSEIL, DARS2, DBNDD2, DDIT4, DEFB108B, DHODH, DNAJB9, DNAJC5B, DPM3, DTNB, EIF4A2, ERP29, ESM1, EXOC4, FAM122B, FANCL, FMNL2, FUBP1, GGA2, GHRH, GLUL, GPN3, HBE1, HELB, HEMK1, INPP5B, KCNJ10, L3MBTL1, LHFPL1, LIPT1, MAGED1, MBOAT1, MDM1, MERTK, METTL3, METTL7B, MGAT4A, MMD, MPI, MRM1, MSH6, MSI2, MSL2, NAPB, NBPF1, NDUFAF3, NLK, NT5DC3, OLIG2, OMA1, OXNAD1, P4HA1, PDIA4, PGBD2, PHF6, PIP5KlA, PMS2, POLR3K, PREPL, RAB3GAP2, RBM39, RBM45, RNF2, RRN3, SEC24A, SFXN2, SIGLEC11, SLC30A3, SNAPC3, SPAG4, SPIN3, SRPRB, SRSF9, STRBP, STX16, SYS1, TAFIA, TGM2, THOC2, TMEM182, TMEM81, TOP1, TP53BP1, TRIM5, TRNT1, TRPM2, UBFD1, URB2, VRK3, WDR76, WDSUB1, XPO1, ZMYND8, ZNF189, ZNF26, ZNF337, ZNF544, ZNF550, ZNF572, and ZNF841.

TABLE 1
Up-Regulated Genes in Metastatic Melanoma

| | | | Entrez | |
|---|---|---|---|---|
| Gene | | UniProtKB | Gene | |
| Symbol | Ensembl ID | ID | ID | Gene Name |
| ABHD10 | ENSG00000144827.5 | Q9NUJ1 | 55347 | abhydrolase domain containing 10 |
| ABHD3 | ENSG00000158201.10 | Q8WU67 | 171586 | abhydrolase domain containing 3 |
| ACVR2B | ENSG00000114739.3 | Q13705 | 93 | activin A receptor, type IIB |
| ADAL | ENSG00000168803.8 | Q6DHV7 | 161823 | adenosine deaminase-like |
| ALG13 | ENSG00000101901.41 | Q9NP73 | 55849 | ALG13, UDP-N- |
| | | | | acetylglucosaminyltransferase subunit |
| ANGEL1 | ENSG00000013523.6 | Q9UNK9 | 23357 | angel homolog 1 (Drosophila) |
| ATG16L1 | ENSG00000085978.18 | Q676U5 | 55054 | autophagy related 16-like 1 (S. |
| | | | | cerevisiae) |
| B4GALT3 | ENSG00000158850.13 | O60512 | 8703 | UDP-Gal:betaGlcNAc beta 1,4- |
| | | | | galactosyltransferase, polypeptide 3 |
| BRAF | ENSG00000157764.21 | P15056 | 673 | v-raf murine sarcoma viral oncogene |
| | | | | homolog B |
| BRSK1 | ENSG00000160469.8 | Q8TDC3 | 84446 | BR serine/threonine kinase 1 |
| C12orf60 | ENSG00000182993.4 | Q5U649 | 144608 | chromosome 12 opon reading frame 60 |
| C1orf56 | ENSG00000143443.3 | Q9BUN1 | 54964 | chromosome 1 open reading frame 56 |
| C4A | ENSG00000244731.17 | P0C0L4 | 720 | complement component 4A (Rodgers |
| | | | | blood group) |
| C7 | ENSG00000112936.7 | P10643 | 730 | complement component 7 |
| CCDC151 | ENSG00000198003.5 | ASD8V7 | 115948 | coiled-coil domain containing 151 |
| CCDC93 | ENSG00000125633.9 | Q567U6 | 54520 | coiled-coil domain containing 93 |
| CCNE1 | ENSG00000105173.7 | P24864 | 898 | cyclin E1 |
| CD1D | ENSG00000158473.1 | P15813 | 912 | CD1d molecule |
| CD38 | ENSG00000004468.5 | P28907 | 952 | CD38 molecule |
| CD5L | ENSG00000073754.2 | O43866 | 922 | CD5 molecule-like |
| CDC42SE1 | ENSG00000197622.7 | Q9NRR8 | 56882 | CDC42 small effector 1 |
| CHEK2 | ENSG00000183765.24 | O96017 | 11200 | checkpoint kinase 2 |
| CHORDC1 | ENSG00000110172.11 | Q9UHD1 | 26973 | cysteine and histidine-rich domain |
| | | | | (CHORD) containing 1 |
| CMTM7 | ENSG00000153551.6 | Q96FZ5 | 112616 | CKLF-like MARVEL transmembrane |
| | | | | domain containing 7 |
| CPOX | ENSG00000080819.5 | P36551 | 1371 | coproporphyrinogen oxidase |
| CR1 | ENSG00000203710.12 | P17927 | 1378 | complement component (3 b/4 b) receptor |
| | | | | 1 (Knops blood group) |
| CRELD1 | ENSG00000163703.11 | Q96HD1 | 78987 | cysteine-rich with EGF-like domains 1 |
| CRNKL1 | ENSG00000101343.7 | Q9BZJ0 | 51340 | crooked neck pre-mRNA splicing factor |
| | | | | 1 |
| CSE1L | ENSG00000124207.3 | P55060 | 1434 | CSE1 chromosome segregation 1-like |
| | | | | (yeast) |
| DARS2 | ENSG00000117593.3 | Q6PI48 | 55157 | aspartyl-tRNA synthetase 2, |
| | | | | mitochondrial |
| DBNDD2 | ENSG00000244274.9 | Q9BQY9 | 55861 | dysbindin (dystrobrevin binding protein |
| | | | | 1) domain containing 2 |
| DDIT4 | ENSG00000168209.3 | Q9NX09 | 54541 | DNA-damage-inducible transcript 4 |
| DEFB108B | ENSG00000184276.2 | Q8NET1 | 245911 | defensin, beta 108B |
| DHODH | ENSG00000102967.9 | Q02127 | 1723 | dihydroorotate dehydrogenase (quinone) |
| DNAJB9 | ENSG00000128590.3 | Q9UBS3 | 4189 | DnaJ (Hsp40) homolog, subfamily B, |
| | | | | member 9 |
| DNAJC5B | ENSG00000147570.4 | Q9UF47 | 85479 | DnaJ (Hsp40) homolog, subfamily C, |
| | | | | member 5 beta |
| DPM3 | ENSG00000179085.3 | Q9P2X0 | 54344 | dolichyl-phosphate mannosyltransferase |
| | | | | polypeptide 3 |
| DTNB | ENSG00000138101.30 | O60941 | 1838 | dystrobrevin, beta |
| EIF4A2 | ENSG00000156976.27 | Q14240 | 1974 | eukaryotic translation initiation factor |
| | | | | 4A2 |
| ERP29 | ENSG00000089248.5 | P30040 | 10961 | endoplasmic reticulum protein 29 |
| ESM1 | ENSG00000164283.4 | Q9NQ30 | 11082 | endothelial cell-specific molecule 1 |
| EXOC4 | ENSG00000131558.24 | Q96A65 | 60412 | exocyst complex component 4 |
| FAM122B | ENSG00000156504.8 | Q7Z309 | 159090 | family with sequence similarity 122B |
| FANCL | ENSG00000115392.10 | Q9NW38 | 55120 | Fanconi anemia, complementation group |
| | | | | L |
| FMNL2 | ENSG00000157827.4 | Q96PY5 | 114793 | formin-like 2 |
| FUBP1 | ENSG00000162613.13 | Q96AE4 | 8880 | far upstream element (FUSE) binding |
| | | | | protein 1 |
| GGA2 | ENSG00000103365.17 | Q9UJY4 | 23062 | golgi-associated, gamma adaptin car |
| | | | | containing, ARF binding protein 2 |
| GHRH | ENSG00000118702.3 | P01286 | 2691 | growth hormone releasing hormone |
| GLUL | ENSG00000135821.13 | P15104 | 2752 | glutamate-ammonia ligase |
| GPN3 | ENSG00000111231.10 | Q9UHWS | 51184 | GPN-loop GTPase 3 |
| HBE1 | ENSG00000213931.3 | P02100 | 3046 | hemoglobin, epsilon 1 |
| HELB | ENSG00000127311.6 | Q8NG08 | 92797 | helicase (DNA) B |
| HEMK1 | ENSG00000114735.7 | Q9Y5R4 | 51409 | HemK methyltransferase family member |
| | | | | 1 |
| INPP5B | ENSG00000204084.11 | P32019 | 3633 | inositol polyphosphate-5-phosphatase, |
| | | | | 75 kDa |
| KCNJ10 | ENSG00000177807.11 | P78508 | 3766 | potassium inwardly-rectifying channel, |
| | | | | subfamily J, member 10 |
| L3MBTL1 | ENSG00000185513.19 | Q9Y468 | 26013 | 1(3)mbt-like 1 (Drosophila) |
| LHFPL1 | ENSG00000182508.2 | Q86WI0 | 10185 | lipoma HMGIC fusion partner-like 1 |
| LIPT1 | ENSG00000144182.7 | Q9Y234 | 51601 | lipoyltransferase 1 |
| MAGED1 | ENSG00000179222.10 | Q9Y5V3 | 9500 | melanoma antigen family D, 1 |
| MBOAT1 | ENSG00000172197.1 | Q6ZNC8 | 154141 | membrane bound O-acyltransferase |
| | | | | domain containing 1 |
| MDM1 | ENSG00000111554.15 | Q8TC05 | 56890 | Mdm1 nuclear protein homolog (mouse) |
| MERTK | ENSG00000153208.7 | Q12866 | 10461 | c-mer proto-oncogene tyrosine kinase |
| METTL3 | ENSG00000165819.14 | Q86U44 | 56339 | methyltransferase like 3 |
| METTL7B | ENSG00000170439.2 | Q6UX53 | 196410 | methyltransferase like 7B |
| MGAT4A | ENSG00000071073.9 | Q9UM21 | 11320 | mannosyl (alpha-1,3-)-glycoprotein beta- |
| | | | | 1,4-N-acetylglucosaminyltransferase, |
| | | | | isozyme A |
| MMD | ENSG00000108960.3 | Q15546 | 23531 | monocyte to macrophage differentiation- |
| | | | | associated |
| MPI | ENSG00000178802.24 | P34949 | 4351 | mannose phosphate isomerase |
| MRM1 | ENSG00000278619.2 | Q6IN84 | 79922 | mitochondrial rRNA methyltransferase 1 |
| | | | | homolog (S. cerevisiae) |
| MSH6 | ENSG00000116062.15 | P52701 | 2956 | mutS homolog 6 |
| MSI2 | ENSG00000153944.19 | Q96DH6 | 124540 | musashi RNA-binding protein 2 |
| MSL2 | ENSG00000174579.5 | Q9HCI7 | 55167 | male-specific lethal 2 homolog |
| | | | | (Drosophila) |
| NAPB | ENSG00000125814.7 | Q9H115 | 8162 | N-ethylmaleimide-sensitive factor |
| | | | | attachment protein, beta |
| NBPF1 | ENSG00000219481.7 | Q3BBV0 | 55672 | neuroblastoma breakpoint family, |
| | | | | member 1 |
| NDUFAF3 | ENSG00000178057.6 | Q9BU61 | 25915 | NADH dehydrogenase (ubiquinone) |
| | | | | complex I, assembly factor 3 |
| NLK | ENSG00000087095.6 | Q9UBE8 | 51701 | nemo-like kinase |
| NT5DC3 | ENSG00000111696.5 | Q86UY8 | 51559 | 5′-nucleotidase domain containing 3 |
| OLIG2 | ENSG00000205927.3 | Q13516 | 10215 | oligodendrocyte lineage transcription |
| | | | | factor 2 |
| OMA1 | ENSG00000162600.11 | Q96E52 | 115209 | OMA1 zinc metallopeptidase |
| OXNAD1 | ENSG00000154814.9 | Q96HP4 | 92106 | oxidoreductase NAD-binding domain |
| | | | | containing 1 |
| P4HA1 | ENSG00000122884.6 | P13674 | 5033 | prolyl 4-hydroxylase, alpha polypeptide I |
| PDIA4 | ENSG00000155660.3 | P13667 | 9601 | protein disulfide isomerase family A, |
| | | | | member 4 |
| PGBD2 | ENSG00000185220.3 | Q6P3X8 | 267002 | piggyBac transposable element derived 2 |
| PHF6 | ENSG00000156531.5 | Q8IWS0 | 84295 | PHD finger protein 6 |
| PIP5K1A | ENSG00000143398.17 | Q99755 | 8394 | phosphatidylinositol-4-phosphate 5- |
| | | | | kinase, type I, alpha |
| PMS2 | ENSG00000122512.11 | P54278 | 5395 | PMS2 postmeiotic segregation increased |
| | | | | 2 (S. cerevisiae) |
| POLR3K | ENSG00000161980.2 | Q9Y2Y1 | 51728 | polymerase (RNA) III (DNA directed) |
| | | | | polypeptide K, 12.3 kDa |
| PREPL | ENSG00000138078.16 | Q4J6C6 | 9581 | prolyl endopeptidase-like |
| RAB3GAP2 | ENSG00000118873.10 | Q9H2M9 | 25782 | RAB3 GTPase activating protein subunit |
| | | | | 2 (non-catalytic) |
| RBM39 | ENSG00000131051.45 | Q14498 | 9584 | RNA binding motif protein 39 |
| RBM45 | ENSG00000155636.7 | Q8IUH3 | 129831 | RNA binding motif protein 45 |
| RNF2 | ENSG00000121481.4 | Q99496 | 6045 | ring finger protein 2 |
| RRN3 | ENSG00000085721.6 | Q9NYV6 | 54700 | RRN3 RNA polymerase I transcription |
| | | | | factor homolog (S. cerevisiae) |
| SEC24A | ENSG00000113615.3 | O95486 | 10802 | SEC24 family member A |
| SFXN2 | ENSG00000156398.13 | Q96NB2 | 94082 | sideroflexin 2 |
| SIGLEC11 | ENSG00000161640.3 | Q96RL6 | 114132 | sialic acid binding Ig-like lectin 11 |
| SLC30A3 | ENSG00000115194.10 | Q99726 | 7781 | solute carrier family 30 (zinc transporter), |
| | | | | member 3 |
| SNAPC3 | ENSG00000164975.7 | Q92966 | 6619 | small nuclear RNA activating complex, |
| | | | | polypeptide 3, 50 kDa |
| SPAG4 | ENSG00000061656.7 | Q9NPE6 | 6676 | sperm associated antigen 4 |
| SPIN3 | ENSG00000204271.45 | Q5JUX0 | 169981 | spindlin family, member 3 |
| SRPRB | ENSG00000144867.5 | Q9Y5M8 | 58477 | signal recognition particle receptor, B |
| | | | | subunit |
| SRSF9 | ENSG00000111786.6 | Q13242 | 8683 | serine/arginine-rich splicing factor 9 |
| STRBP | ENSG00000165209.10 | Q96SI9 | 55342 | spermatid perinuclear RNA binding |
| | | | | protein |
| STX16 | ENSG00000124222.20 | O14662 | 8675 | syntaxin 16 |
| SYS1 | ENSG00000204070.8 | Q8N2H4 | 90196 | SYS1 Golgi-localized integral membrane |
| | | | | protein homolog (S. cerevisiae) |
| TAF1A | ENSG00000143498.7 | Q15573 | 9015 | TATA box binding protein (TBP)- |
| | | | | associated factor, RNA polymerase I, A, |
| | | | | 48 kDa |
| TGM2 | ENSG00000198959.6 | P21980 | 7052 | transglutaminase 2 |
| THOC2 | ENSG00000125676.20 | Q8NI27 | 57187 | THO complex 2 |
| TMEM182 | ENSG00000170417.11 | Q6ZP80 | 130827 | transmembrane protein 182 |
| TMEM81 | ENSG00000174529.1 | Q6P7N7 | 388730 | transmembrane protein 81 |
| TOP1 | ENSG00000198900.1 | P11387 | 7150 | topoisomerase (DNA) I |
| TP53BP1 | ENSG00000067369.17 | Q12888 | 7158 | tumor protein p53 binding protein 1 |
| TRIM5 | ENSG00000132256.11 | Q9C035 | 85363 | tripartite motif containing 5 |
| TRNT1 | ENSG00000072756.11 | Q96Q11 | 51095 | tRNA nucleotidyl transferase, CCA- |
| | | | | adding, 1 |
| TRPM2 | ENSG00000142185.8 | O94759 | 7226 | transient receptor potential cation |
| | | | | channel, subfamily M, member 2 |
| UBFD1 | ENSG00000103353.10 | O14562 | 56061 | ubiquitin family domain containing 1 |
| URB2 | ENSG00000135763.2 | Q14146 | 9816 | URB2 ribosome biogenesis 2 homolog |
| | | | | (S. cerevisiae) |
| VRK3 | ENSG00000105053.26 | Q8IV63 | 51231 | vaccinia related kinase 3 |
| WDR76 | ENSG00000092470.4 | Q9H967 | 79968 | WD repeat domain 76 |
| WDSUB1 | ENSG00000196151.5 | Q8N9V3 | 151525 | WD repeat, sterile alpha motif and U-box |
| | | | | domain containing 1 |
| XPO1 | ENSG00000082898.24 | O14980 | 7514 | exportin 1 |
| ZMYND8 | ENSG00000101040.21 | Q9ULU4 | 23613 | zinc finger, MYND-type containing 8 |
| ZNF189 | ENSG00000136870.4 | O75820 | 7743 | zinc finger protein 189 |
| ZNF26 | ENSG00000198393.7 | P17031 | 7574 | zinc finger protein 26 |
| ZNF337 | ENSG00000130684.3 | Q9Y3M9 | 26152 | zinc finger protein 337 |
| ZNF544 | ENSG00000198131.20 | Q6NX49 | 27300 | zinc finger protein 544 |
| ZNF550 | ENSG00000251369.8 | Q7Z398 | 162972 | zinc finger protein 550 |
| ZNF572 | ENSG00000180938.1 | Q7Z317 | 137209 | zinc finger protein 572 |
| ZNF841 | ENSG00000197608.7 | Q6ZN19 | 284371 | zinc finger protein 841 |

### Down-Regulated Genes in Metastatic Melanoma

In one embodiment, the invention provides the identification of a genome wide tumor derived gene expression based biomarker that is associated with poor prognosis in melanoma. In a further embodiment, the invention provides a set of 513 genes whose expression is negatively correlated with identifying a patient with poor prognosis for treating early stage melanoma. In yet a further embodiment, the invention comprises a gene expression based biomarker comprising down-regulated genes, wherein the gene expression based biomarker comprises genes listed in Table 2. In a sub-embodiment, the invention provides the identification of a gene expression based biomarker that allows classification of a patient into a prognosis group, wherein the prognosis group is predictive of a patient's need of treatment. In another sub-embodiment, the invention relates to the identification of a genome wide tumor derived gene expression based biomarker that can be used in identifying, classifying, and/or treating melanoma patients with early disease (Stage 0 or Stage I).

In one embodiment, the invention provides a gene expression based biomarker comprising at least 5 genes listed in Table 2 that are negatively correlated with a need of further treatment for a patient who has been diagnosed with melanoma. In one embodiment, a patient is identified as a patient with poor prognosis if the patient has a lower expression of down-regulated genes listed in Table 2. In one embodiment, a patient is identified as a patient with good prognosis if the patient has a higher expression of down-regulated genes listed in Table 2.

In one embodiment, the invention provides a method of using a gene expression based biomarker to identify melanoma patients with a poor prognosis in early stage disease.

In one embodiment, the invention provides a method of treating a melanoma patient with early stage disease by identification of the patient with a gene expression based biomarker. In another embodiment, the invention provides a method of identifying melanoma patients who have metastatic melanoma versus primary melanoma. In yet a further embodiment, the invention relates to identification of a patient with a decreased level of gene expression based biomarker, wherein the gene expression based biomarker comprises 5 or more down-regulated genes from Table 2, and wherein the patient has metastatic melanoma, to evaluate for further treatment options.

In some embodiments, the invention relates to identifying a melanoma patient having a poor prognosis. In a sub-embodiment, the patient having a poor prognosis is likely to have a reoccurrence of melanoma, metastatic disease progression, or poor overall survival.

In some embodiments, the melanoma is early stage. In another embodiment, the melanoma is primary melanoma. In another embodiment, the melanoma is metastatic melanoma.

In some embodiments, the invention relates to classifying a patient having a favorable prognosis based on a gene expression level by calculating an elevated level of a gene expression of 5 or more down-regulated genes listed in Table 2 or classifying a patient having a poor prognosis based on a gene expression level by calculating a decreased level of a gene expression of 5 or more down-regulated genes listed in table 2. A further sub-embodiment is to classify a patient as having either a favorable prognosis or a poor prognosis based on a gene expression level by calculating a level of gene expression of 5 or more down-regulated genes listed in Table 2, wherein a lower gene expression level of down-regulated genes indicates a patient with a poor prognosis and a patient likely to have pathology related to metastatic melanoma and a higher gene expression level of down-regulated genes indicates a patient with a favorable prognosis and a patient not likely to have pathology related to metastatic melanoma. A patient is positive for a gene expression based biomarker if the patient has a lower expression of at least 5 of the down-regulated genes listed in Table 2. As a result, the tumor is classified as biomarker positive and the patient is in need of further treatment.

In a sub-embodiment, the invention relates to classifying a patient as having a poor prognosis based on a gene expression level by calculating a lower level of gene expression of 5 or more down-regulated genes listed in Table 2. Additionally, the invention relates to the calculation of a lower level of gene expression used in determining a threshold for patients in a clinical trial setting. A further sub-embodiment comprises changing the threshold based on clinical outcomes designated for the clinical trial.

In one embodiment, the invention relates to selecting those melanoma patients having a poor prognosis based on having a low expression of 5 or more down-regulated genes listed in Table 2 for clinical trials to evaluate a patient's need of treatment and to facilitate efficacious treatments and therapies for patients with an unmet clinical need. The invention further relates to selecting those patients having a poor prognosis based on having a low expression of down-regulated genes listed in Table 2 for clinical trials in order to effectively evaluate a new treatment method.

In one embodiment, the invention relates to classifying a patient having a favorable prognosis based on a gene expression level by calculating an elevated level of gene expression of 5 or more down-regulated genes listed in Table 2.

In one embodiment, the invention relates to identifying biomarkers within a sample obtained from a patient, e.g., a patient's tumor to calculate a gene signature score.

In a further embodiment, the invention relates to calculating a gene signature score based on the down-regulated genes to predict a prognosis for a melanoma patient. In a further aspect, the classification of a prognosis for a melanoma patient allows for treatment with an appropriate treatment option. A patient having a favorable prognosis may not have a clinical need for additional treatment and can avoid possible side effects.

In one embodiment, the invention relates to the use of a gene expression based biomarker signature score for a gene expression based biomarker which comprises a set of at least about 5 of the down-regulated genes listed in Table 2 to identify a patient with a favorable prognosis or a poor prognosis, based on gene expression level of the signature score.

In particular embodiments, the gene expression based biomarker comprises at least 5 (five) genes selected from the genes listed in Table 2. In other embodiments, the gene expression based biomarker comprises at least 6 genes, at least 7 genes, at least 8 genes, at least 9 genes, at least 10 genes, at least 11 genes, at least 12 genes, at least 13 genes, etc. or 513 genes from the genes listed in Table 2.

In a particular embodiment, the gene expression based biomarker comprises the following genes: A4GALT, ABLIM1, ADAM15, ADAM33, ADAMTS12, ADAMTS2, ADAMTS5, ADK, AGTR1, AHNAK, AHNAK2, AKR1C1, AKR1C2, AKR1C3, ALDH3A1, ALDH3B2, ALOXE3, ALS2CL, ANGPTL2, ANO1, ANPEP, ANXA2, ANXA9, APCDD1, APLNR, AQP1, AQP3, AQP5, ARHGEF15, ARHGEF19, ARHGEF4, ARL4D, ARNTL2, ASAP3, ASPN, ASPRV1, ATL3, ATP12A, ATP6V1C2, ATP8B1, B3GNT4, BDKRB2, BICC1, BICD2, BMP1, BMPR2, BOC, BSPRY, BTBD11, C12orf54, C19orf33, CA12, CALML3, CALML5, CAPN1, CAPNS2, CASZ1, CBLC, CCDC113, CCDC120, CCDC3, CCDC92, CCL22, CD109, CD24, CD248, CD34, CD44, CD9, CDA, CDH13, CDH3, CDHR1, CDR1, CDS1, CEACAM19, CH25H, CLDN1, CLDN4, CLEC14A, CLIC3, CLTB, CNFN, COL12A1, COL3A1, COL14A1, COL15A1, COL17A1, COL18A1, COL1A1, COL1A2, COL23A1, COL3A1, COL5A1, COL5A2, COL5A3, COL6A1, COL6A2, COL6A3, COL6A6, COL7A1, COL8A2, COMP, COMTD1, CPA3, CPA4, CPXM1, CPXM2, CPZ, CRABP1, CRABP2, CRCT1, CREB3L1, CRISPLD2, CRYM, CST6, CSTA, CTNNBIP1, CTSG, CTSK, CTTNBP2NL, CXADR, CXCL12, CXCL14, CYB561, CYB5R3, CYP26B1, CYP2S1, CYYR1, DAPL1, DAZAP2, DCN, DEGS1, DEGS2, DENND2C, DGAT2, DHRS1, DIO2, DMKN, DPP4, DPT, DSC2, DSEL, DSP, DST, DUOX1, DUOXA1, DUSP14, EBF1, ECSCR, EDN1, EFNA3, EFNB2, EGLN3, EHD2, ELMO3, ELOVL3, ELOVL4, ELOVL7, EML1, EMP1, EMP2, EN1, EPHA1, EPHB6, EPHX3, EPPK1, EPS8L1, ERBB2, ESRP2, ETS2, EVPL, EXPH5, F10, F2RL1, F2RL2, FADS6, FAM110C, FAM167A, FAM180A, FAM83F, FAM83H, FAT2, FAT4, FBLN1, FBLN2, FBN1, FCERIA, FGF11, FGFR3, FIBIN, FMO1, FOSL2, FOXQ1, FUT1, FZD10, GALNT1, GAN, GAS1, GDPD3, GJA1, GJB2, GJB3, GJB5, GJB6, GLT8D2, GLTP, GNA15, GNAL, GPC1, GPR68, GREM1, GRHL1, GRHL2, GSDMA, HAS3, HDC, HEBP2, HES2, HOPX, HOXD10, HR, HSDI 1B2, HSPAI2B, HTRA1, ID1, IDE, IFFO2, IGFBP4, IGFL2, IGFL4, ILIR1, ILIRN, IL20RB, IMPA2, IRX2, IRX3, IRX5, ISM1, ITGB4, IVL, JAM2, JMJD7-, LA2G4B, JUP, KCND3, KCNK6, KCNK7, KCTD11, KIAA1217, KIAA1522, KIF26A, KIT, KITLG, KLC3, KLF10, KLF11, KLF3, KLF4, KLF5, KLF6, KLK10, KLK5, KLK6, KLK8, KRT1, KRT10, KRT15, KRT17, KRT19, KRT2, KRT23, KRT31, KRT5, KRT78, KRT79, KRT80, KRTAP10-12, KRTDAP, LAD1, LAMA2, LAMA3, LAMB3, LCEIA, LCEIB, LCEID, LCE1F, LCE2A, LCE3A, LCN2, LIMA1, LOXL1, LRRC15, LRRC32, LRRC8E, LTB4R, LTBP1, LUM, LY6D, LY6G6C, LYNX1, LYPD2, LYPD3, LYPD5, MAL2, MALL, MAP7, MARVELD1, MAST4, MEGF6, MEOX1, MFAP4, MFAP5, MICALL1, MINK1, MMP11, MMP2, MMP7, MMRN2, MN1, MPZL2, MRGPRF, MSX2, MXRA5, MXRA8, MYO6, NCCRP1, NDRG4, NDUFA4L2, NEURLIB, NFATC4, NGEF, NIPAL4, NKD2, NLRX1, NMU, NRARP, NTF3, NTN1, NUAK1, OLFM2, OLFML1, OLFML2A, OSR2, OTUB2, OVOL1, PAK6, PALLD, PALMD, PAPPA, PAQR7, PCDH18, PDE2A, PDGFRA, PDGFRB, PDGFRL, PDLIM1, PDPN, PDZKIIP1, PERP, PI16, PI3, PKP1, PKP3, PLA2G4F, PLCH2, PLEC, PLEK2, PLEKHA1, PLIN3, PLP2, PLVAP, PLXDC1, PMFBP1, PPL, PPP1R13L, PPPIR14C, PPP2R3A, PPP4R1, PRG2, PROM2, PRRX1, PRRX2, PRSS22, PRSS27, PRSS3, PRSS8, PSAPLI, PTGES, PTGS1, PTPN21, PTPRF, PYDC1, RAB25, RAB3D, RAET1G, RAPGEFL1, RASAL1, RDH12, RHBG, RHCG, RHOD, RIMS3, RIN1, ROBO4, RORA, RPS6KA4, RSPO1, S100A14, S100A16, S100A2, S100A7, S100A8, S100A9, SBSN, SCNNIA, SDC1, SDCBP2, SDK1, SELP, SERPINB8, SFN, SFRP2, SFTPD, SGPP2, SH2D3A, SH3D19, SH3GL1, SIX2, SLC22A23, SLC24A3, SLC30A1, SLC47A2, SLC6A9, SLCO2A1, SLIT3, SLPI, SLURP1, SMAD1, SMAGP, SMPD3, SNAI2, SNX7, SORBS3, SOX15, SOX18, SOX7, SP6, SPARC, SPINT1, SPINT2, SPNS2, SPON1, SPRRIB, SPRR2D, SPRR2E, SPRR2F, SPRR4, SPTLC3, SSH3, ST14, STAB2, STEAP4, STMN2, STON2, SULT2B1, TACSTD2, TAX1BP3, TBX15, TFCP2L1, TGM1, TGM5, THBD, THRB, TMEM119, TMEM154, TMEM30B, TMEM45A, TMEM79, TMTC3, TNFAIP8L3, TNKSIBP1, TNXB, TP53AIP1, TP63, TPBG, TPPP3, TRIM7, TSHZ3, TSPANI1, TSPAN18, TSPO, TUBA4A, TUFT1, TWIST2, TYRP1, UNC5B, VASN, VDR, VGI13, VSIG10L, WFDC12, WNT11, WNT3, WNT4, WNT5A, XG, ZBTB7C, ZC3H12A, ZNF185, ZNF296, ZNF385A, ZNF423, and ZNF521.

TABLE 2
Down-Regulated Genes in Metastatic Melanoma

| | | | Entrez | |
|---|---|---|---|---|
| Gene | | UniProtKB | Gene | |
| Symbol | Ensembl ID | ID | ID | Gene Name |
| A4GALT | ENSG00000128274.7 | Q9NPC4 | 53947 | alpha 1,4-galactosyltransferase |
| ABLIM1 | ENSG00000099204.14 | O14639 | 3983 | actin binding LIM protein 1 |
| ADAM15 | ENSG00000143537.32 | Q13444 | 8751 | ADAM metallopeptidase domain 15 |
| ADAM33 | ENSG00000149451.7 | Q9BZ11 | 80332 | ADAM metallopeptidase domain 33 |
| ADAMTS12 | ENSG00000151388.6 | P58397 | 81792 | ADAM metallopeptidase with thrombospondin |
| | | | | type 1 motif, 12 |
| ADAMTS2 | ENSG00000087116.5 | O95450 | 9509 | ADAM metallopeptidase with thrombospondin |
| | | | | type 1 motif, 2 |
| ADAMTS5 | ENSG00000154736.1 | Q9UNA0 | 11096 | ADAM metallopeptidase with thrombospondin |
| | | | | type 1 motif, 5 |
| ADK | ENSG00000156110.6 | P55263 | 132 | adenosine kinase |
| AGTR1 | ENSG00000144891.9 | P30556 | 185 | angiotensin II receptor, type 1 |
| AHNAK | ENSG00000124942.8 | Q09666 | 195 | AHNAK nucleoprotein |
| AHNAK2 | ENSG00000185567.4 | Q8IVF2 | 113146 | AHNAK nucleoprotein 2 |
| AKR1C1 | ENSG00000187134.5 | Q04828 | 1645 | aldo-keto reductase family 1, member C1 |
| AKR1C2 | ENSG00000151632.10 | P52895 | 1646 | aldo-keto reductase family 1, member C2 |
| AKR1C3 | ENSG00000196139.11 | P42330 | 8644 | aldo-keto reductase family 1, member C3 |
| ALDH3A1 | ENSG00000108602.17 | P30838 | 218 | aldehyde dehydrogenase 3 family, member A1 |
| ALDH3B2 | ENSG00000132746.8 | P48448 | 222 | aldehyde dehydrogenase 3 family, member B2 |
| ALOXE3 | ENSG00000179148.4 | Q9BYJ1 | 59344 | arachidonate lipoxygenase 3 |
| ALS2CL | ENSG00000178038.10 | Q60I27 | 259173 | ALS2 C-terminal like |
| ANGPTL2 | ENSG00000136859.4 | Q9UKU9 | 23452 | angiopoietin-like 2 |
| ANO1 | ENSG00000131620.10 | Q5XXA6 | 55107 | anoctamin 1, calcium activated chloride channel |
| ANPEP | ENSG00000166825.9 | P15144 | 290 | alanyl (membrane) aminopeptidase |
| ANXA2 | ENSG00000182718.38 | P07355 | 302 | annexin A2 |
| ANXA9 | ENSG00000143412.2 | O76027 | 8416 | annexin A9 |
| APCDD1 | ENSG00000154856.6 | Q8J025 | 85500 | adenomatosis polyposis coli down-regulated 1 |
| APLNR | ENSG00000134817.3 | P35414 | 187 | apelin receptor |
| AQP1 | ENSG00000240583.5 | P29972 | 358 | aquaporin 1 (Colton blood group) |
| AQP3 | ENSG00000165272.7 | Q92482 | 360 | aquaporin 3 (Gill blood group) |
| AQP5 | ENSG00000161798.2 | P55064 | 362 | aquaporin 5 |
| ARHGEF15 | ENSG00000198844.8 | O94989 | 22899 | Rho guanine nucleotide exchange factor (GEF) 15 |
| ARHGEF19 | ENSG00000142632.6 | Q8IW93 | 128272 | Rho guanine nucleotide exchange factor (GEF) 19 |
| ARHGEF4 | ENSG00000136002.18 | Q9NR80 | 50649 | Rho guanine nucleotide exchange factor (GEF) 4 |
| ARL4D | ENSG00000175906.1 | P49703 | 379 | ADP-ribosylation factor-like 4D |
| ARNTL2 | ENSG00000029153.9 | Q8WYA1 | 56938 | aryl hydrocarbon receptor nuclear |
| | | | | translocator-like 2 |
| ASAP3 | ENSG00000088280.12 | Q8TDY4 | 55616 | ArfGAP with SH3 domain, ankyrin repeat and PH |
| | | | | domain 3 |
| ASPN | ENSG00000106819.2 | Q9BXN1 | 54829 | asporin |
| ASPRV1 | ENSG00000244617.1 | Q53RT3 | 151516 | aspartic peptidase, retroviral-like 1 |
| ATL3 | ENSG00000184743.4 | Q6DD88 | 25923 | atlastin GTPase 3 |
| ATP12A | ENSG00000075673.2 | P54707 | 479 | ATPase, H+/K+ transporting, nongastric, alpha |
| | | | | polypeptide |
| ATP6V1C2 | ENSG00000143882.4 | Q8NEY4 | 245973 | ATPase, H+ transporting, lysosomal 42 kDa, V1 |
| | | | | subunit C2 |
| ATP8B1 | ENSG00000081923.6 | O43520 | 5205 | ATPase, aminophospholipid transporter, class I, |
| | | | | type 8B, member 1 |
| B3GNT4 | ENSG00000176383.6 | Q9C0J1 | 79369 | UDP-GlcNAc:betaGal beta-1,3-N- |
| | | | | acetylglucosaminyltransferase 4 |
| BDKRB2 | ENSG00000168398.3 | P30411 | 624 | bradykinin receptor B2 |
| BICC1 | ENSG00000122870.3 | Q9H694 | 80114 | BicC family RNA binding protein 1 |
| BICD2 | ENSG00000185963.2 | Q8TD16 | 23299 | bicaudal D homolog 2 (Drosophila) |
| BMP1 | ENSG00000168487.18 | P13497 | 649 | bone morphogenetic protein 1 |
| BMPR2 | ENSG00000204217.4 | Q13873 | 659 | bone morphogenetic protein receptor, type II |
| | | | | (serine/threonine kinase) |
| BOC | ENSG00000144857.17 | Q9BWV1 | 91653 | BOC cell adhesion associated, oncogene regulated |
| BSPRY | ENSG00000119411.2 | Q5W0U4 | 54836 | B-box and SPRY domain containing |
| BTBD11 | ENSG00000151136.7 | A6QL63 | 121551 | BTB (POZ) domain containing 11 |
| C12orf54 | ENSG00000177627.6 | Q6X4T0 | 121273 | chromosome 12 open reading frame 54 |
| C19orf33 | ENSG00000167644.4 | Q9GZP8 | 64073 | chromosome 19 open reading frame 33 |
| CA12 | ENSG00000074410.6 | O43570 | 771 | carbonic anhydrase XII |
| CALML3 | ENSG00000178363.1 | P27482 | 810 | calmodulin-like 3 |
| CALML5 | ENSG00000178372.1 | Q9NZT1 | 51806 | calmodulin-like 5 |
| CAPN1 | ENSG00000014216.28 | P07384 | 823 | calpain 1, (mu/I) large subunit |
| CAPNS2 | ENSG00000256812.1 | Q96L46 | 84290 | calpain, small subunit 2 |
| CASZ1 | ENSG00000130940.9 | Q86V15 | 54897 | castor zinc finger 1 |
| CBLC | ENSG00000142273.4 | Q9ULV8 | 23624 | Cbl proto-oncogene C, E3 ubiquitin protein ligase |
| CCDC113 | ENSG00000103021.6 | Q9H0I3 | 29070 | coiled-coil domain containing 113 |
| CCDC120 | ENSG00000147144.8 | Q96HBS | 90060 | coiled-coil domain containing 120 |
| CCDC3 | ENSG00000151468.2 | Q9BQI4 | 83643 | coiled-coil domain containing 3 |
| CCDC92 | ENSG00000119242.9 | Q53HC0 | 80212 | coiled-coil domain containing 92 |
| CCL22 | ENSG00000102962.1 | O00626 | 6367 | chemokine (C-C motif) ligand 22 |
| CD109 | ENSG00000156535.4 | Q6YHK3 | 135228 | CD109 molecule |
| CD24 | ENSG00000272398.8 | P25063 | 934 | CD24 molecule |
| CD248 | ENSG00000174807.1 | Q9HCU0 | 57124 | CD248 molecule, endosialin |
| CD34 | ENSG00000174059.4 | P28906 | 947 | CD34 molecule |
| CD44 | ENSG00000026508.39 | P16070 | 960 | CD44 molecule (Indian blood group) |
| CD9 | ENSG00000010278.16 | P21926 | 928 | CD9 molecule |
| CDA | ENSG00000158825.2 | P32320 | 978 | cytidine deaminase |
| CDH13 | ENSG00000140945.14 | P55290 | 1012 | cadherin 13 |
| CDH3 | ENSG00000062038.10 | P22223 | 1001 | cadherin 3, type 1, P-cadherin (placental) |
| CDHR1 | ENSG00000148600.7 | Q96JP9 | 92211 | cadherin-related family member 1 |
| CDR1 | ENSG00000184258.1 | N/A | 1038 | cerebellar degeneration-related protein 1, 34 kDa |
| CDS1 | ENSG00000163624.2 | Q92903 | 1040 | CDP-diacylglycerol synthase (phosphatidate |
| | | | | cytidylyltransferase) 1 |
| CEACAM19 | ENSG00000186567.9 | Q7Z692 | 56971 | carcinoembryonic antigen-related cell adhesion |
| | | | | molecule 19 |
| CH25H | ENSG00000138135.1 | O95992 | 9023 | cholesterol 25-hydroxylase |
| CLDN1 | ENSG00000163347.2 | O95832 | 9076 | claudin 1 |
| CLDN4 | ENSG00000189143.5 | O14493 | 1364 | claudin 4 |
| CLEC14A | ENSG00000176435.1 | Q86T13 | 161198 | C-type lectin domain family 14, member A |
| CLIC3 | ENSG00000169583.3 | O95833 | 9022 | chloride intracellular channel 3 |
| CLTB | ENSG00000175416.7 | P09497 | 1212 | clathrin, light chain B |
| CNFN | ENSG00000105427.2 | Q9BYD5 | 84518 | cornifelin |
| COL12A1 | ENSG00000111799.11 | Q99715 | 1303 | collagen, type XII, alpha 1 |
| COL13A1 | ENSG00000197467.15 | Q5TAT6 | 1305 | collagen, type XIII, alpha 1 |
| COL14A1 | ENSG00000187955.9 | Q05707 | 7373 | collagen, type XIV, alpha 1 |
| COL15A1 | ENSG00000204291.5 | P39059 | 1306 | collagen, type XV, alpha 1 |
| COL17A1 | ENSG00000065618.7 | Q9UMD9 | 1308 | collagen, type XVII, alpha 1 |
| COL18A1 | ENSG00000182871.7 | P39060 | 80781 | collagen, type XVIII, alpha 1 |
| COL1A1 | ENSG00000108821.13 | P02452 | 1277 | collagen, type I, alpha 1 |
| COL1A2 | ENSG00000164692.13 | P08123 | 1278 | collagen, type I. alpha 2 |
| COL23A1 | ENSG00000050767.4 | Q86Y22 | 91522 | collagen, type XXIII, alpha 1 |
| COL3A1 | ENSG00000168542.6 | P02461 | 1281 | collagen, type III, alpha 1 |
| COL5A1 | ENSG00000130635.8 | P20908 | 1289 | collagen, type V, alpha 1 |
| COL5A2 | ENSG00000204262.3 | P05997 | 1290 | collagen, type V, alpha 2 |
| COL5A3 | ENSG00000080573.2 | P25940 | 50509 | collagen, type V, alpha 3 |
| COL6A1 | ENSG00000142156.7 | P12109 | 1291 | collagen, type VI, alpha 1 |
| COL6A2 | ENSG00000142173.8 | P12110 | 1292 | collagen, type VI, alpha 2 |
| COL6A3 | ENSG00000163359.14 | P12111 | 1293 | collagen, type VI, alpha 3 |
| COL6A6 | ENSG00000206384.3 | A6NMZ7 | 131873 | collagen, type VI, alpha 6 |
| COL7A1 | ENSG00000114270.10 | Q02388 | 1294 | collagen, type VII, alpha 1 |
| COL8A2 | ENSG00000171812.3 | P25067 | 1296 | collagen, type VIII, alpha 2 |
| COMP | ENSG00000105664.4 | P49747 | 1311 | cartilage oligomeric matrix protein |
| COMTD1 | ENSG00000165644.7 | Q86VU5 | 118881 | catechol-O-methyltransferase domain containing 1 |
| CPA3 | ENSG00000163751.2 | P15088 | 1359 | carboxypeptidase A3 (mast cell) |
| CPA4 | ENSG00000128510.11 | Q9UI42 | 51200 | carboxypeptidase A4 |
| CPXM1 | ENSG00000088882.1 | Q96SM3 | 56265 | carboxypeptidase X (M14 family), member 1 |
| CPXM2 | ENSG00000121898.4 | Q8N436 | 119587 | carboxypeptidase X (M14 family), member 2 |
| CPZ | ENSG00000109625.9 | Q66K79 | 8532 | carboxypeptidase Z |
| CRABP1 | ENSG00000166426.3 | P29762 | 1381 | cellular retinoic acid binding protein 1 |
| CRABP2 | ENSG00000143320.4 | P29373 | 1382 | cellular retinoic acid binding protein 2 |
| CRCT1 | ENSG00000169509.1 | Q9UGL9 | 54544 | cysteine-rich C-terminal 1 |
| CREB3L1 | ENSG00000157613.6 | Q96BA8 | 90993 | cAMP responsive element binding protein 3-like 1 |
| CRISPLD2 | ENSG00000103196.11 | Q9H0B8 | 83716 | cysteine-rich secretory protein LCCL domain |
| | | | | containing 2 |
| CRYM | ENSG00000103316.10 | Q14894 | 1428 | crystallin, mu |
| CST6 | ENSG00000175315.1 | Q15828 | 1474 | cystatin E/M |
| CSTA | ENSG00000121552.2 | P01040 | 1475 | cystatin A (stefin A) |
| CTNNBIP1 | ENSG00000178585.4 | Q9NSA3 | 56998 | catenin, beta interacting protein 1 |
| CTSG | ENSG00000100448.2 | P08311 | 1511 | cathepsin G |
| CTSK | ENSG00000143387.3 | P43235 | 1513 | cathepsin K |
| CTTNBP2NL | ENSG00000143079.4 | Q9P2B4 | 55917 | CTTNBP2 N-terminal like |
| CXADR | ENSG00000154639.5 | P78310 | 1525 | coxsackie virus and adenovirus receptor |
| CXCL12 | ENSG00000107562.8 | P48061 | 6387 | chemokine (C-X-C motif) ligand 12 |
| CXCL14 | ENSG00000145824.2 | O95715 | 9547 | chemokine (C-X-C motif) ligand 14 |
| CYB561 | ENSG00000008283.21 | P49447 | 1534 | cytochrome b561 |
| CYB5R3 | ENSG00000100243.8 | P00387 | 1727 | cytochrome b5 reductase 3 |
| CYP26B1 | ENSG00000003137.5 | Q9NR63 | 56603 | cytochrome P450, family 26, subfamily B, |
| | | | | polypeptide 1 |
| CYP2S1 | ENSG00000167600.6 | Q96SQ9 | 29785 | cytochrome P450, family 2, subfamily S, |
| | | | | polypeptide 1 |
| CYYR1 | ENSG00000166265.2 | Q96J86 | 94038 | cysteine/tyrosine-rich 1 |
| DAPL1 | ENSG00000163331.4 | A0PJW8 | 92196 | death associated protein-like 1 |
| DAZAP2 | ENSG00000183283.15 | Q15038 | 9802 | DAZ associated protein 2 |
| DCN | ENSG00000011465.20 | P07585 | 1634 | decorin |
| DEGS1 | ENSG00000143753.5 | O15121 | 8560 | delta(4)-desaturase, sphingolipid 1 |
| DEGS2 | ENSG00000168350.3 | Q6QHC5 | 123099 | delta(4)-desaturase, sphingolipid 2 |
| DENND2C | ENSG00000175984.6 | Q68D51 | 163259 | DENN/MADD domain containing 2C |
| DGAT2 | ENSG00000062282.9 | Q96PD7 | 84649 | diacylglycerol O-acyltransferase 2 |
| DHRS1 | ENSG00000157379.11 | Q96LJ7 | 115817 | dehydrogenase/reductase (SDR family) member 1 |
| DIO2 | ENSG00000211448.11 | Q92813 | 1734 | deiodinase, iodothyronine, type II |
| DMKN | ENSG00000161249.68 | Q6E0U4 | 93099 | dermokine |
| DPP4 | ENSG00000197635.11 | P27487 | 1803 | dipeptidyl-peptidase 4 |
| DPT | ENSG00000143196.1 | Q07507 | 1805 | dermatopontin |
| DSC2 | ENSG00000134755.2 | Q02487 | 1824 | desmocollin 2 |
| DSEL | ENSG00000171451.1 | Q8IZU8 | 92126 | dermatan sulfate epimerase-like |
| DSP | ENSG00000096696.3 | P15924 | 1832 | desmoplakin |
| DST | ENSG00000151914.35 | Q03001 | 667 | dystonin |
| DUOX1 | ENSG00000137857.12 | Q9NRD9 | 53905 | dual oxidase 1 |
| DUOXA1 | ENSG00000140254.16 | Q1HG43 | 90527 | dual oxidase maturation factor 1 |
| DUSP14 | ENSG00000276023.3 | O95147 | 11072 | dual specificity phosphatase 14 |
| EBF1 | ENSG00000164330.11 | Q9UH73 | 1879 | early B-cell factor 1 |
| ECSCR | ENSG00000249751.1 | Q19T08 | 641700 | endothelial cell surface expressed |
| | | | | chemotaxis and apoptosis regulator |
| EDN1 | ENSG00000078401.1 | P05305 | 1906 | endothelin 1 |
| EFNA3 | ENSG00000143590.3 | P52797 | 1944 | ephrin-A3 |
| EFNB2 | ENSG00000125266.3 | P52799 | 1948 | ephrin-B2 |
| EGLN3 | ENSG00000129521.11 | Q9H6Z9 | 112399 | egl-9 family hypoxia-inducible factor 3 |
| EHD2 | ENSG00000024422.5 | Q9NZN4 | 30846 | EH-domain containing 2 |
| ELMO3 | ENSG00000102890.5 | Q96BJ8 | 79767 | engulfment and cell motility 3 |
| ELOVL3 | ENSG00000119915.1 | Q9HB03 | 83401 | ELOVL fatty acid elongase 3 |
| ELOVL4 | ENSG00000118402.1 | Q9GZR5 | 6785 | ELOVL fatty acid elongase 4 |
| ELOVL7 | ENSG00000164181.7 | A1L3X0 | 79993 | ELOVL fatty acid elongase 7 |
| EML1 | ENSG00000066629.20 | O00423 | 2009 | echinoderm microtubule associated protein like 1 |
| EMP1 | ENSG00000134531.12 | P54849 | 2012 | epithelial membrane protein 1 |
| EMP2 | ENSG00000213853.3 | P54851 | 2013 | epithelial membrane protein 2 |
| EN1 | ENSG00000163064.2 | Q05925 | 2019 | engrailed homeobox 1 |
| EPHA1 | ENSG00000146904.7 | P21709 | 2041 | EPH receptor A1 |
| EPHB6 | ENSG00000106123.11 | N/A | 2051 | EPH receptor B6 |
| EPHX3 | ENSG00000105131.4 | Q9H6B9 | 79852 | epoxide hydrolase 3 |
| EPPK1 | ENSG00000261150.2 | P58107 | 83481 | epiplakin 1 |
| EPS8L1 | ENSG00000131037.19 | Q8TE68 | 54869 | EPS8-like 1 |
| ERBB2 | ENSG00000141736.22 | P04626 | 2064 | v-erb-b2 avian erythroblastic leukemia viral |
| | | | | oncogene homolog 2 |
| ESRP2 | ENSG00000103067.12 | Q9H6T0 | 80004 | epithelial splicing regulatory protein 2 |
| ETS2 | ENSG00000157557.4 | P15036 | 2114 | v-ets avian erythroblastosis virus E26 oncogene |
| | | | | homolog 2 |
| EVPL | ENSG00000167880.5 | Q92817 | 2125 | envoplakin |
| EXPH5 | ENSG00000110723.6 | Q8NEV8 | 23086 | exophilin 5 |
| F10 | ENSG00000126218.7 | P00742 | 2159 | coagulation factor X |
| F2RL1 | ENSG00000164251.2 | P55085 | 2150 | coagulation factor II (thrombin) receptor-like 1 |
| F2RL2 | ENSG00000164220.2 | O00254 | 2151 | coagulation factor II (thrombin) receptor-like 2 |
| FADS6 | ENSG00000172782.5 | N/A | 283985 | fatty acid desaturase 6 |
| FAM110C | ENSG00000184731.3 | Q1W6H9 | 642273 | family with sequence similarity 110, member C |
| FAM167A | ENSG00000154319.7 | Q96KS9 | 83648 | family with sequence similarity 167, member A |
| FAM180A | ENSG00000189320.4 | Q6UWF9 | 389558 | family with sequence similarity 180, member A |
| FAM83F | ENSG00000133477.3 | Q8NEG4 | 113828 | family with sequence similarity 83, member F |
| FAM83H | ENSG00000180921.2 | Q6ZRV2 | 286077 | family with sequence similarity 83, member H |
| FAT2 | ENSG00000086570.2 | Q9NYQ8 | 2196 | FAT atypical cadherin 2 |
| FAT4 | ENSG00000196159.3 | Q6V0I7 | 79633 | FAT atypical cadherin 4 |
| FBLN1 | ENSG00000077942.18 | P23142 | 2192 | fibulin 1 |
| FBLN2 | ENSG00000163520.7 | P98095 | 2199 | fibulin 2 |
| FBN1 | ENSG00000166147.8 | P35555 | 2200 | fibrillin 1 |
| FCER1A | ENSG00000179639.2 | P12319 | 2205 | Fc fragment of IgE, high affinity I, receptor |
| | | | | for; alpha polypeptide |
| FGF11 | ENSG00000161958.6 | Q92914 | 2256 | fibroblast growth factor 11 |
| FGFR3 | ENSG00000068078.11 | P22607 | 2261 | fibroblast growth factor receptor 3 |
| FIBIN | ENSG00000176971.1 | Q8TAL6 | 387758 | fin bud initiation factor homolog (zebrafish) |
| FMO1 | ENSG00000010932.8 | Q01740 | 2326 | flavin containing monooxygenase 1 |
| FOSL2 | ENSG00000075426.4 | P15408 | 2355 | FOS-like antigen 2 |
| FOXQ1 | ENSG00000164379.1 | Q9C009 | 94234 | forkhead box Q1 |
| FUT1 | ENSG00000174951.7 | P19526 | 2523 | fucosyltransferase 1 (galactoside 2-alpha-L- |
| | | | | fucosyltransferase, H blood group) |
| FZD10 | ENSG00000111432.2 | Q9ULW2 | 11211 | frizzled class receptor 10 |
| GALNT1 | ENSG00000141429.6 | Q10472 | 2589 | polypeptide N-acetylgalactosaminyltransferase 1 |
| GAN | ENSG00000261609.2 | Q9H2C0 | 8139 | gigaxonin |
| GAS1 | ENSG00000180447.1 | P54826 | 2619 | growth arrest-specific 1 |
| GDPD3 | ENSG00000102886.5 | Q7L5L3 | 79153 | glycerophosphodiester phosphodiesterase domain |
| | | | | containing 3 |
| GJA1 | ENSG00000152661.1 | P17302 | 2697 | gap junction protein, alpha 1, 43 kDa |
| GJB2 | ENSG00000165474.3 | P29033 | 2706 | gap junction protein, beta 2, 26 kDa |
| GJB3 | ENSG00000188910.2 | O75712 | 2707 | gap junction protcin, beta 3, 31 kDa |
| GJB5 | ENSG00000189280.1 | O95377 | 2709 | gap junction protein, beta 5, 31.1 kDa |
| GJB6 | ENSG00000121742.15 | O95452 | 10804 | gap junction protein, beta 6, 30 kDa |
| GLT8D2 | ENSG00000120820.7 | Q9H1C3 | 83468 | glycosyltransferase 8 domain containing 2 |
| GLTP | ENSG00000139433.6 | Q9NZD2 | 51228 | glycolipid transfer protein |
| GNA15 | ENSG00000060558.3 | P30679 | 2769 | guanine nucleotide binding protein (G protein), |
| | | | | alpha 15 (Gg class) |
| GNAL | ENSG00000141404.10 | P38405 | 2774 | guanine nucleotide binding protein (G protein), |
| | | | | alpha activating activity polypeptide, |
| | | | | olfactory type |
| GPC1 | ENSG00000063660.9 | P35052 | 2817 | glypican 1 |
| GPR68 | ENSG00000119714.4 | Q15743 | 8111 | G protein-coupled receptor 68 |
| GREM1 | ENSG00000166923.3 | O60565 | 26585 | gremlin 1, DAN family BMP antagonist |
| GRHL1 | ENSG00000134317.8 | Q9NZI5 | 29841 | grainyhead-like 1 (Drosophila) |
| GRHL2 | ENSG00000083307.7 | Q6ISB3 | 79977 | grainyhead-like 2 (Drosophila) |
| GSDMA | ENSG00000167914.3 | Q96QA5 | 284110 | gasdermin A |
| HAS3 | ENSG00000103044.5 | O00219 | 3038 | hyaluronan synthase 3 |
| HDC | ENSG00000140287.7 | P19113 | 3067 | histidine decarboxylase |
| HEBP2 | ENSG00000051620.4 | Q9Y5Z4 | 23593 | heme binding protein 2 |
| HES2 | ENSG00000069812.6 | Q9Y543 | 54626 | hes family bHLH transcription factor 2 |
| HOPX | ENSG00000171476.15 | Q9BPY8 | 84525 | HOP homeobox |
| HOXD10 | ENSG00000128710.3 | P28358 | 3236 | homeobox D10 |
| HR | ENSG00000168453.9 | O43593 | 3264 | hair growth associated |
| HSD11B2 | ENSG00000176387.4 | P80365 | 3291 | hydroxysteroid (11-beta) dehydrogenase 2 |
| HSPA12B | ENSG00000132622.2 | Q96MM6 | 116835 | heat shock 70 kD protein 12B |
| HTRA1 | ENSG00000166033.2 | Q92743 | 5654 | HtrA serine peptidase 1 |
| ID1 | ENSG00000125968.2 | P41134 | 3397 | inhibitor of DNA binding 1, dominant negative |
| | | | | helix-loop-helix protein |
| IDE | ENSG00000119912.8 | P14735 | 3416 | insulin-degrading enzyme |
| IFFO2 | ENSG00000169991.3 | QSTF58 | 126917 | intermediate filament family orphan 2 |
| IGFBP4 | ENSG00000141753.1 | P22692 | 3487 | insulin-like growth factor binding protein 4 |
| IGFL2 | ENSG00000204866.5 | Q6UWQ7 | 147920 | IGF-like family member 2 |
| IGFL4 | ENSG00000204869.4 | Q6B9Z1 | 444882 | IGF-like family member 4 |
| IL1R1 | ENSG00000115594.13 | P14778 | 3554 | interleukin 1 receptor, type I |
| ILIRN | ENSG00000136689.9 | P18510 | 3557 | interleukin 1 receptor antagonist |
| IL20RB | ENSG00000174564.5 | Q6UXL0 | 53833 | interleukin 20 receptor beta |
| IMPA2 | ENSG00000141401.12 | O14732 | 3613 | inositol(myo)-1(or 4)-monophosphatase 2 |
| IRX2 | ENSG00000170561.2 | Q9BZI1 | 93965 | iroquois homeobox 2 |
| IRX3 | ENSG00000177508.3 | P78415 | 79191 | iroquois homeobox 3 |
| IRX5 | ENSG00000176842.5 | P78411 | 10265 | iroquois homeobox 5 |
| ISM1 | ENSG00000101230.1 | B1AKI9 | 140862 | isthmin 1, angiogenesis inhibitor |
| ITGB4 | ENSG00000132470.13 | P16144 | 3691 | integrin, beta 4 |
| IVL | ENSG00000163207.1 | P07476 | 3713 | involucrin |
| JAM2 | ENSG00000154721.7 | P57087 | 58494 | junctional adhesion molecule 2 |
| JMJD7- | ENSG00000168970.6 | N/A | 8681 | JMJD7-PLA2G4B readthrough |
| PLA2G4B | | | | |
| JUP | ENSG00000173801.12 | P14923 | 3728 | junction plakoglobin |
| KCND3 | ENSG00000171385.3 | Q9UK17 | 3752 | potassium voltage-gated channel, Shal-related |
| | | | | subfamily, member 3 |
| KCNK6 | ENSG00000099337.2 | Q9Y257 | 9424 | potassium channel, subfamily K, member 6 |
| KCNK7 | ENSG00000173338.6 | Q9Y2U2 | 10089 | potassium channel, subfamily K, member 7 |
| KCTD11 | ENSG00000213859.2 | Q693B1 | 147040 | potassium channel tetramerization domain |
| | | | | containing 11 |
| KIAA1217 | ENSG00000120549.17 | Q5T5P2 | 56243 | + |
| KIAA1522 | ENSG00000162522.5 | Q9P206 | 57648 | + |
| KIF26A | ENSG00000066735.2 | Q9ULI4 | 26153 | kinesin family member 26A |
| KIT | ENSG00000157404.4 | P10721 | 3815 | v-kit Hardy-Zuckerman 4 feline sarcoma viral |
| | | | | oncogene homolog |
| KITLG | ENSG00000049130.7 | P21583 | 4254 | KIT ligand |
| KLC3 | ENSG00000104892.7 | Q6P597 | 147700 | kinesin light chain 3 |
| KLF10 | ENSG00000155090.2 | Q13118 | 7071 | Kruppel-like factor 10 |
| KLF11 | ENSG00000172059.6 | O14901 | 8462 | Kruppel-like factor 11 |
| KLF3 | ENSG00000109787.3 | P57682 | 51274 | Kruppel-like factor 3 (basic) |
| KLF4 | ENSG00000136826.6 | O43474 | 9314 | Kruppel-like factor 4 (gut) |
| KLF5 | ENSG00000102554.5 | Q13887 | 688 | Kruppel-like factor 5 (intestinal) |
| KLF6 | ENSG00000067082.7 | Q99612 | 1316 | Kruppel-like factor 6 |
| KLK10 | ENSG00000129451.6 | O43240 | 5655 | kallikrein-related peptidase 10 |
| KLK5 | ENSG00000167754.5 | Q9Y337 | 25818 | kallikrein-related peptidase 5 |
| KLK6 | ENSG00000167755.7 | Q92876 | 5653 | kallikrein-related peptidase 6 |
| KLK8 | ENSG00000129455.11 | O60259 | 11202 | kallikrein-related peptidase 8 |
| KRT1 | ENSG00000167768.2 | P04264 | 3848 | keratin 1 |
| KRT10 | ENSG00000186395.2 | P13645 | 3858 | keratin 10 |
| KRT15 | ENSG00000171346.9 | P19012 | 3866 | keratin 15 |
| KRT17 | ENSG00000128422.7 | Q04695 | 3872 | keratin 17 |
| KRT19 | ENSG00000171345.7 | P08727 | 3880 | keratin 19 |
| KRT2 | ENSG00000172867.2 | P35908 | 3849 | keratin 2 |
| KRT23 | ENSG00000108244.9 | Q9C075 | 25984 | keratin 23 (histone deacetylase inducible) |
| KRT31 | ENSG00000094796.1 | Q15323 | 3881 | keratin 31 |
| KRT5 | ENSG00000186081.11 | P13647 | 3852 | keratin 5 |
| KRT78 | ENSG00000170423.5 | Q8N1N4 | 196374 | keratin 78 |
| KRT79 | ENSG00000185640.3 | Q5XKE5 | 338785 | keratin 79 |
| KRT80 | ENSG00000167767.3 | Q6KB66 | 144501 | keratin 80 |
| KRTAP10-12 | ENSG00000189169.2 | P60413 | 386685 | keratin associated protein 10-12 |
| KRTDAP | ENSG00000188508.3 | P60985 | 94035 | keratinocyte differentiation-associated protein |
| LAD1 | ENSG00000159166.8 | O00515 | 3898 | ladinin 1 |
| LAMA2 | ENSG00000196569.7 | P24043 | 3908 | laminin, alpha 2 |
| LAMA3 | ENSG00000053747.14 | Q16787 | 3909 | laminin, alpha 3 |
| LAMB3 | ENSG00000196878.5 | Q13751 | 3914 | laminin, beta 3 |
| LCE1A | ENSG00000186844.1 | Q5T7P2 | 353131 | late cornified envelope 1A |
| LCE1B | ENSG00000196734.1 | Q5T7P3 | 353132 | late cornified envelope 1B |
| LCE1D | ENSG00000172155.1 | Q5T752 | 353134 | late cornified envelope 1D |
| LCE1F | ENSG00000240386.1 | Q5T754 | 353137 | late cornified envelope 1F |
| LCE2A | ENSG00000187173.1 | Q5TA79 | 353139 | late cornified envelope 2A |
| LCE3A | ENSG00000185962.1 | Q5TA76 | 353142 | late cornified envelope 3A |
| LCN2 | ENSG00000148346.7 | P80188 | 3934 | lipocalin 2 |
| LIMA1 | ENSG00000050405.16 | Q9UHB6 | 51474 | LIM domain and actin binding 1 |
| LOXL1 | ENSG00000129038.5 | Q08397 | 4016 | lysyl oxidase-like 1 |
| LRRC15 | ENSG00000172061.2 | Q8TF66 | 131578 | leucine rich repeat containing 15 |
| LRRC32 | ENSG00000137507.5 | Q14392 | 2615 | leucine rich repeat containing 32 |
| LRRC8E | ENSG00000171017.6 | Q6NSJ5 | 80131 | leucine rich repeat containing 8 family, member E |
| LTB4R | ENSG00000213903.5 | Q15722 | 1241 | leukotriene B4 receptor |
| LTBP1 | ENSG00000049323.12 | Q14766 | 4052 | latent transforming growth factor beta binding |
| | | | | protein 1 |
| LUM | ENSG00000139329.3 | P51884 | 4060 | lumican |
| LY6D | ENSG00000167656.4 | Q14210 | 8581 | lymphocyte antigen 6 complex, locus D |
| LY6G6C | ENSG00000204421.2 | O95867 | 80740 | lymphocyte antigen 6 complex, locus G6C |
| LYNX1 | ENSG00000180155.6 | PODP58 | 66004 | Ly6/neurotoxin 1 |
| LYPD2 | ENSG00000197353.1 | Q6UXB3 | 137797 | LY6/PLAUR domain containing 2 |
| LYPD3 | ENSG00000124466.4 | O95274 | 27076 | LY6/PLAUR domain containing 3 |
| LYPD5 | ENSG00000159871.8 | Q6UWN5 | 284348 | LY6/PLAUR domain containing 5 |
| MAL2 | ENSG00000147676.4 | Q969L2 | 114569 | mal. T-cell differentiation protein 2 |
| | | | | (gene/pseudogene) |
| MALL | ENSG00000144063.3 | Q13021 | 4119 | mal, T-cell differentiation protein-like |
| MAP7 | ENSG00000135525.9 | Q14244 | 9053 | microtubule-associated protein 7 |
| MARVELD1 | ENSG00000155254.4 | Q9BSK0 | 83742 | MARVEL domain containing 1 |
| MAST4 | ENSG00000069020.21 | O15021 | 23227 | microtubule associated serine/threonine kinase |
| | | | | family member 4 |
| MEGF6 | ENSG00000162591.7 | O75095 | 1953 | multiple EGF-like-domains 6 |
| MEOX1 | ENSG00000005102.4 | P50221 | 4222 | mesenchyme homeobox 1 |
| MFAP4 | ENSG00000166482.5 | P55083 | 4239 | microfibrillar-associated protein 4 |
| MFAP5 | ENSG00000197614.15 | Q13361 | 8076 | microfibrillar associated protein 5 |
| MICALL1 | ENSG00000100139.6 | Q8N3F8 | 85377 | MICAL-like 1 |
| MINK1 | ENSG00000141503.13 | Q8N4C8 | 50488 | misshapen-like kinase 1 |
| MMP11 | ENSG00000099953.13 | P24347 | 4320 | matrix metallopeptidase 11 (stromelysin 3) |
| MMP2 | ENSG00000087245.8 | P08253 | 4313 | matrix metallopeptidase 2 (gelatinase A, 72 kDa |
| | | | | gelatinase, 72 kDa type IV collagenase) |
| MMP7 | ENSG00000137673.3 | P09237 | 4316 | matrix metallopeptidase 7 (matrilysin, uterine) |
| MMRN2 | ENSG00000173269.7 | Q9H8L6 | 79812 | multimerin 2 |
| MN1 | ENSG00000169184.3 | Q10571 | 4330 | meningioma (disrupted in balanced translocation) 1 |
| MPZL2 | ENSG00000149573.7 | O60487 | 10205 | myelin protein zero-like 2 |
| MRGPRF | ENSG00000172935.3 | Q96AM1 | 116535 | MAS-related GPR, member F |
| MSX2 | ENSG00000120149.2 | P35548 | 4488 | msh homeobox 2 |
| MXRA5 | ENSG00000101825.1 | Q9NR99 | 25878 | matrix-remodelling associated 5 |
| MXRA8 | ENSG00000162576.10 | Q9BRK3 | 54587 | matrix-remodelling associated 8 |
| MYO6 | ENSG00000196586.8 | Q9UM54 | 4646 | myosin VI |
| NCCRP1 | ENSG00000188505.1 | Q6ZVX7 | 342897 | non-specific cytotoxic cell receptor protein 1 |
| | | | | homolog (zebrafish) |
| NDRG4 | ENSG00000103034.55 | Q9ULP0 | 65009 | NDRG family member 4 |
| NDUFA4L2 | ENSG00000185633.7 | Q9NRX3 | 56901 | NADH dehydrogenase (ubiquinone) 1 alpha |
| | | | | subcomplex, 4-like 2 |
| NEURL1B | ENSG00000214357.3 | A8MQ27 | 54492 | neuralized E3 ubiquitin protein ligase 1B |
| NFATC4 | ENSG00000100968.33 | Q14934 | 4776 | nuclear factor of activated T-cells, cytoplasmic, |
| | | | | calcineurin-dependent 4 |
| NGEF | ENSG00000066248.9 | Q8N5V2 | 25791 | neuronal guanine nucleotide exchange factor |
| NIPAL4 | ENSG00000172548.5 | Q0D2K0 | 348938 | NIPA-like domain containing 4 |
| NKD2 | ENSG00000145506.5 | Q969F2 | 85409 | naked cuticle homolog 2 (Drosophila) |
| NLRX1 | ENSG00000160703.14 | Q86UT6 | 79671 | NLR family member XI |
| NMU | ENSG00000109255.6 | P48645 | 10874 | neuromedin U |
| NRARP | ENSG00000198435.1 | Q7Z6K4 | 441478 | NOTCH-regulated ankyrin repeat protein |
| NTF3 | ENSG00000185652.6 | P20783 | 4908 | neurotrophin 3 |
| NTN1 | ENSG00000065320.2 | O95631 | 9423 | netrin 1 |
| NUAK1 | ENSG00000074590.4 | O60285 | 9891 | NUAK family, SNF1-like kinase, 1 |
| OLFM2 | ENSG00000105088.5 | O95897 | 93145 | olfactomedin 2 |
| OLFML1 | ENSG00000183801.5 | Q6UWY5 | 283298 | olfactomedin-like 1 |
| OLFML2A | ENSG00000185585.3 | Q68BL7 | 169611 | olfactomedin-like 2A |
| OSR2 | ENSG00000164920.10 | Q8N2R0 | 116039 | odd-skipped related transciption factor 2 |
| OTUB2 | ENSG00000089723.2 | Q96DC9 | 78990 | OTU deubiquitinase, ubiquitin aldehyde binding 2 |
| OVOL1 | ENSG00000172818.3 | O14753 | 5017 | ovo-like zinc finger 1 |
| PAK6 | ENSG00000137843.17 | Q9NQU5 | 56924 | p21 protein (Cdc42/Rac)-activated kinase 6 |
| PALLD | ENSG00000129116.17 | Q8WX93 | 23022 | palladin, cytoskeletal associated protein |
| PALMD | ENSG00000099260.5 | Q9NP74 | 54873 | palmdelphin |
| PAPPA | ENSG00000182752.3 | Q13219 | 5069 | pregnancy-associated plasma protein A, |
| | | | | pappalysin 1 |
| PAQR7 | ENSG00000182749.1 | Q86WK9 | 164091 | progestin and adipoQ receptor family member VII |
| PCDH18 | ENSG00000189184.8 | Q9HCL0 | 54510 | protocadherin 18 |
| PDE2A | ENSG00000186642.27 | O00408 | 5138 | phosphodiesterase 2A, cGMP-stimulated |
| PDGFRA | ENSG00000134853.10 | P16234 | 5156 | platelet-derived growth factor receptor, alpha |
| | | | | polypeptide |
| PDGFRB | ENSG00000113721.11 | P09619 | 5159 | platelet-derived growth factor receptor, beta |
| | | | | polypeptide |
| PDGFRL | ENSG00000104213.3 | Q15198 | 5157 | platelet-derived growth factor receptor-like |
| PDLIM1 | ENSG00000107438.5 | O00151 | 9124 | PDZ and LIM domain 1 |
| PDPN | ENSG00000162493.12 | Q86YL7 | 10630 | podoplanin |
| PDZK1IP1 | ENSG00000162366.4 | Q13113 | 10158 | PDZKI interacting protein 1 |
| PERP | ENSG00000112378.1 | Q96FX8 | 64065 | PERP, TP53 apoptosis effector |
| PI16 | ENSG00000164530.3 | Q6UXB8 | 221476 | peptidase inhibitor 16 |
| PI3 | ENSG00000124102.1 | P19957 | 5266 | peptidase inhibitor 3, skin-derived |
| PKP1 | ENSG00000081277.5 | Q13835 | 5317 | plakophilin 1 (ectodermal dysplasia/skin fragility |
| | | | | syndrome) |
| PKP3 | ENSG00000184363.11 | Q9Y446 | 11187 | plakophilin 3 |
| PLA2G4F | ENSG00000168907.7 | Q68DD2 | 255189 | phospholipasc A2, group IVF |
| PLCH2 | ENSG00000149527.8 | O75038 | 9651 | phospholipase C, eta 2 |
| PLEC | ENSG00000178209.15 | Q15149 | 5339 | plectin |
| PLEK2 | ENSG00000100558.6 | Q9NYT0 | 26499 | pleckstrin 2 |
| PLEKHA1 | ENSG00000107679.8 | Q9HB21 | 59338 | pleckstrin homology domain containing, family A |
| | | | | (phosphoinositide binding specific) member 1 |
| PLIN3 | ENSG00000105355.7 | O60664 | 10226 | perilipin 3 |
| PLP2 | ENSG00000102007.2 | Q04941 | 5355 | proteolipid protein 2 (colonic epithelium-enriched) |
| PLVAP | ENSG00000130300.3 | Q9BX97 | 83483 | plasmalemma vesicle associated protein |
| PLXDC1 | ENSG00000161381.20 | Q8IUK5 | 57125 | plexin domain containing 1 |
| PMFBP1 | ENSG00000118557.11 | Q8TBY8 | 83449 | polyamine modulated factor 1 binding protein 1 |
| PPL | ENSG00000118898.6 | O60437 | 5493 | periplakin |
| PPP1R13L | ENSG00000104881.9 | Q8WUF5 | 10848 | protein phosphatase 1, regulatory subunit 13 like |
| PPP1R14C | ENSG00000198729.1 | Q8TAE6 | 81706 | protein phosphatase 1, regulatory (inhibitor) |
| | | | | subunit 14C |
| PPP2R3A | ENSG00000073711.5 | Q06190 | 5523 | protein phosphatase 2, regulatory subunit B″, alpha |
| PPP4R1 | ENSG00000154845.29 | Q8TF05 | 9989 | protein phosphatase 4, regulatory subunit 1 |
| PRG2 | ENSG00000186652.4 | P13727 | 5553 | proteoglycan 2, bone marrow (natural killer cell |
| | | | | activator, eosinophil granule major basic protein) |
| PROM2 | ENSG00000155066.11 | Q8N271 | 150696 | prominin 2 |
| PRRX1 | ENSG00000116132.8 | P54821 | 5396 | paired related homeobox 1 |
| PRRX2 | ENSG00000167157.1 | Q99811 | 51450 | paired related homeobox 2 |
| PRSS22 | ENSG00000005001.8 | Q9GZN4 | 64063 | protease, serine, 22 |
| PRSS27 | ENSG00000172382.5 | Q9BQR3 | 83886 | protease, serine 27 |
| PRSS3 | ENSG00000010438.8 | P35030 | 5646 | protease, serine, 3 |
| PRSS8 | ENSG00000052344.6 | Q16651 | 5652 | protease. serine, 8 |
| PSAPL1 | ENSG00000178597.1 | Q6NUJ1 | 768239 | prosaposin-like 1 (gene/pseudogene) |
| PTGES | ENSG00000148344.2 | O14684 | 9536 | prostaglandin E synthase |
| PTGS1 | ENSG00000095303.11 | P23219 | 5742 | prostaglandin-endoperoxide synthase 1 |
| | | | | (prostaglandin G/H synthase and cyclooxygenase) |
| PTPN21 | ENSG00000070778.9 | Q16825 | 11099 | protein tyrosine phosphatase, non-receptor type 21 |
| PTPRF | ENSG00000142949.16 | P10586 | 5792 | protein tyrosine phosphatase, receptor type, F |
| PYDC1 | ENSG00000169900.2 | Q8WXC3 | 260434 | PYD (pyrin domain) containing 1 |
| RAB25 | ENSG00000132698.5 | P57735 | 57111 | RAB25, member RAS oncogene family |
| RAB3D | ENSG00000105514.2 | O95716 | 9545 | RAB3D, member RAS oncogene family |
| RAET1G | ENSG00000203722.3 | Q6H3X3 | 353091 | retinoic acid early transcript 1G |
| RAPGEFL1 | ENSG00000108352.11 | Q9UHV5 | 51195 | Rap guanine nucleotide exchange factor |
| | | | | (GEF)-like 1 |
| RASAL1 | ENSG00000111344.10 | O95294 | 8437 | RAS protein activator like 1 (GAP1 like) |
| RDH12 | ENSG00000139988.4 | Q96NR8 | 145226 | retinol dehydrogenase 12 (all-trans/9-cis/11-cis) |
| RHBG | ENSG00000132677.9 | Q9H310 | 57127 | Rh family, B glycoprotein (gene/pseudogene) |
| RHCG | ENSG00000140519.6 | Q9UBD6 | 51458 | Rh family, C glycoprotein |
| RHOD | ENSG00000173156.3 | O00212 | 29984 | ras homolog family member D |
| RIMS3 | ENSG00000117016.2 | Q9UJD0 | 9783 | regulating synaptic membrane exocytosis 3 |
| RIN1 | ENSG00000174791.8 | Q13671 | 9610 | Ras and Rab interactor 1 |
| ROBO4 | ENSG00000154133.10 | Q8WZ75 | 54538 | roundabout, axon guidance receptor, homolog 4 |
| | | | | (Drosophila) |
| RORA | ENSG00000069667.14 | P35398 | 6095 | RAR-related orphan receptor A |
| RPS6KA4 | ENSG00000162302.8 | O75676 | 8986 | ribosomal protein S6 kinase, 90 kDa, |
| | | | | polypeptide 4 |
| RSPO1 | ENSG00000169218.4 | Q2MKA7 | 284654 | R-spondin 1 |
| S100A14 | ENSG00000189334.6 | Q9HCY8 | 57402 | S100 calcium binding protein A14 |
| S100A16 | ENSG00000188643.5 | Q96FQ6 | 140576 | S100 calcium binding protein A16 |
| S100A2 | ENSG00000196754.6 | P29034 | 6273 | S100 calcium binding protein A2 |
| S100A7 | ENSG00000143556.2 | P31151 | 6278 | S100 calcium binding protein A7 |
| S100A8 | ENSG00000143546.3 | P05109 | 6279 | S100 calcium binding protein A8 |
| S100A9 | ENSG00000163220.1 | P06702 | 6280 | S100 calcium binding protein A9 |
| SBSN | ENSG00000189001.3 | Q6UWP8 | 374897 | suprabasin |
| SCNN1A | ENSG00000111319.23 | P37088 | 6337 | sodium channel, non-voltage-gated 1 alpha subunit |
| SDC1 | ENSG00000115884.6 | P18827 | 6382 | syndecan 1 |
| SDCBP2 | ENSG00000125775.6 | Q9H190 | 27111 | syndecan binding protein (syntenin) 2 |
| SDK1 | ENSG00000146555.10 | Q7Z5N4 | 221935 | sidekick cell adhesion molecule 1 |
| SELP | ENSG00000174175.7 | P16109 | 6403 | selectin P (granule membrane protein 140 kDa, |
| | | | | antigen CD62) |
| SERPINB8 | ENSG00000166401.9 | P50452 | 5271 | serpin peptidase inhibitor, clade B |
| | | | | (ovalbumin), member 8 |
| SFN | ENSG00000175793.1 | P31947 | 2810 | stratifin |
| SFRP2 | ENSG00000145423.1 | Q96HF1 | 6423 | secreted frizzled-related protein 2 |
| SFTPD | ENSG00000133661.2 | P35247 | 6441 | surfactant protein D |
| SGPP2 | ENSG00000163082.1 | Q8IWX5 | 130367 | sphingosine-1-phosphate phosphatase 2 |
| SH2D3A | ENSG00000125731.8 | Q9BRG2 | 10045 | SH2 domain containing 3.A. |
| SH3D19 | ENSG00000109686.13 | Q5HYK7 | 152503 | SH3 domain containing 19 |
| SH3GL1 | ENSG00000141985.6 | Q99961 | 6455 | SH3-domain GRB2-like 1 |
| SIX2 | ENSG00000170577.1 | Q9NPC8 | 10736 | SIX homeobox 2 |
| SLC22A23 | ENSG00000137266.12 | A1A5C7 | 63027 | solute carrier family 22, member 23 |
| SLC24A3 | ENSG00000185052.2 | Q9HC58 | 56225 | solute carrier family 24 (sodium/potassium/ |
| | | | | calcium exchanger), member 3 |
| SLC30A1 | ENSG00000170385.1 | Q9Y6M5 | 7779 | solute carrier family 30 (zinc transporter), |
| | | | | member 1 |
| SLC47A2 | ENSG00000180638.10 | Q86VL8 | 146802 | solute carrier family 47 (multidrug and toxin |
| | | | | extrusion), member 2 |
| SLC6A9 | ENSG00000196517.11 | P48067 | 6536 | solute carrier family 6 (neurotransmitter |
| | | | | transporter, glycine), member 9 |
| SLCO2A1 | ENSG00000174640.8 | Q92959 | 6578 | solute carrier organic anion transporter family, |
| | | | | member 2A1 |
| SLIT3 | ENSG00000184347.8 | O75094 | 6586 | slit homolog 3 (Drosophila) |
| SLPI | ENSG00000124107.1 | P03973 | 6590 | secretory leukocyte peptidase inhibitor |
| SLURP1 | ENSG00000126233.1 | P55000 | 57152 | secreted LY6/PLAUR domain containing 1 |
| SMAD1 | ENSG00000170365.16 | Q15797 | 4086 | SMAD family member 1 |
| SMAGP | ENSG00000170545.8 | Q0VAQ4 | 57228 | small cell adhesion glycoprotein |
| SMPD3 | ENSG00000103056.12 | Q9NY59 | 55512 | sphingomyelin phosphodiesterase 3, neutral |
| | | | | membrane (neutral sphingomyelinase II) |
| SNAI2 | ENSG00000019549.3 | O43623 | 6591 | snail family zinc finger 2 |
| SNX7 | ENSG00000162627.5 | Q9UNH6 | 51375 | sorting nexin 7 |
| SORBS3 | ENSG00000120896.22 | O60504 | 10174 | sorbin and SH3 domain containing 3 |
| SOX15 | ENSG00000129194.3 | O60248 | 6665 | SRY (sex determining region Y)-box 15 |
| SOX18 | ENSG00000203883.1 | P35713 | 54345 | SRY (sex determining region Y)-box 18 |
| SOX7 | ENSG00000171056.1 | Q9BT81 | 83595 | SRY (sex determining region Y)-box 7 |
| SP6 | ENSG00000189120.2 | Q3SY56 | 80320 | Sp6 transcription factor |
| SPARC | ENSG00000113140.9 | P09486 | 6678 | secreted protein, acidic, cysteine-rich (osteonectin) |
| SPINT1 | ENSG00000166145.11 | O43278 | 6692 | serine peptidase inhibitor, Kunitz type 1 |
| SPINT2 | ENSG00000167642.11 | O43291 | 10653 | serine peptidase inhibitor, Kunitz type, 2 |
| SPNS2 | ENSG00000183018.8 | Q8IVW8 | 124976 | spinster homolog 2 (Drosophila) |
| SPON1 | ENSG00000262655.2 | Q9HCB6 | 10418 | spondin 1, extracellular matrix protein |
| SPRR1B | ENSG00000169469.1 | P22528 | 6699 | small proline-rich protein 1B |
| SPRR2D | ENSG00000163216.4 | P22532 | 6703 | small proline-rich protein 2D |
| SPRR2E | ENSG00000203785.2 | P22531 | 6704 | small proline-rich protein 2E |
| SPRR2F | ENSG00000244094.1 | Q96RM1 | 6705 | small proline-rich protein 2F |
| SPRR4 | ENSG00000184148.1 | Q96PI1 | 163778 | small proline-rich protein 4 |
| SPTLC3 | ENSG00000172296.6 | Q9NUV7 | 55304 | serine palmitoyltransferase, long |
| | | | | chain base subunit 3 |
| SSH3 | ENSG00000172830.11 | Q8TE77 | 54961 | slingshot protein phosphatase 3 |
| ST14 | ENSG00000149418.4 | Q9YSY6 | 6768 | suppression of tumorigenicity 14 (colon carcinoma) |
| STAB2 | ENSG00000136011.6 | Q8WWQ8 | 55576 | stabilin 2 |
| STEAP4 | ENSG00000127954.3 | Q687X5 | 79689 | STEAP family member 4 |
| STMN2 | ENSG00000104435.3 | Q93045 | 11075 | stathmin-like 2 |
| STON2 | ENSG00000140022.9 | Q8WXE9 | 85439 | stonin 2 |
| SULT2B1 | ENSG00000088002.4 | 000204 | 6820 | sulfotransferase family, cytosolic, 2B, member 1 |
| TACSTD2 | ENSG00000184292.1 | P09758 | 4070 | tumor-associated calcium signal transducer 2 |
| TAX1BP3 | ENSG00000213977.2 | O14907 | 30851 | Tax1 (human T-cell leukemia virus type I) binding |
| | | | | protein 3 |
| TBX15 | ENSG00000092607.3 | Q96SF7 | 6913 | T-box 15 |
| TFCP2L1 | ENSG00000115112.2 | Q9NZI6 | 29842 | transcription factor CP2-like 1 |
| TGM1 | ENSG00000092295.9 | P22735 | 7051 | transglutaminase 1 |
| TGM5 | ENSG00000104055.8 | O43548 | 9333 | transglutaminase 5 |
| THBD | ENSG00000178726.1 | P07204 | 7056 | thrombomodulin |
| THRB | ENSG00000151090.20 | P10828 | 7068 | thyroid hormone receptor, beta |
| TMEM119 | ENSG00000183160.4 | Q4V9L6 | 338773 | transmembrane protein 119 |
| TMEM154 | ENSG00000170006.4 | Q6P9G4 | 201799 | transmembrane protein 154 |
| TMEM30B | ENSG00000182107.3 | Q3MIR4 | 161291 | transmembrane protein 30B |
| TMEM45A | ENSG00000181458.7 | Q9NWC5 | 55076 | transmembrane protein 45A |
| TMEM79 | ENSG00000163472.7 | Q9BSE2 | 84283 | transmembrane protein 79 |
| TMTC3 | ENSG00000139324.4 | Q6ZXV5 | 160418 | transmembrane and tetratricopeptide repeat |
| | | | | containing 3 |
| TNFAIP8L3 | ENSG00000183578.2 | Q5GJ75 | 388121 | tumor necrosis factor, alpha-induced |
| | | | | protein 8-like 3 |
| TNKS1BP1 | ENSG00000149115.7 | Q9C0C2 | 85456 | tankyrase 1 binding protein 1, 182 kDa |
| TNXB | ENSG00000168477.10 | P22105 | 7148 | tenascin XB |
| TP53AIP1 | ENSG00000120471.6 | Q9HCN2 | 63970 | tumor protein p53 regulated apoptosis inducing |
| | | | | protein 1 |
| TP63 | ENSG00000073282.14 | Q9H3D4 | 8626 | tumor protein p63 |
| TPBG | ENSG00000146242.3 | Q13641 | 7162 | trophoblast glycoprotein |
| TPPP3 | ENSG00000159713.5 | Q9BW30 | 51673 | tubulin polymerization-promoting protein family |
| | | | | member 3 |
| TRIM7 | ENSG00000146054.7 | Q9C029 | 81786 | tripartite motif containing 7 |
| TSHZ3 | ENSG00000121297.3 | Q63HK5 | 57616 | teashirt zinc finger homeobox 3 |
| TSPAN11 | ENSG00000110900.4 | AIL157 | 441631 | tetraspanin 11 |
| TSPAN18 | ENSG00000157570.13 | Q96SJ8 | 90139 | tetraspanin 18 |
| TSPO | ENSG00000100300.6 | P30536 | 706 | translocator protein (18 kDa) |
| TUBA4A | ENSG00000127824.10 | P68366 | 7277 | tubulin, alpha 4a |
| TUFT1 | ENSG00000143367.7 | Q9NNX1 | 7286 | tuftelin 1 |
| TWIST2 | ENSG00000233608.2 | Q8WVJ9 | 117581 | twist family bHLH transcription factor 2 |
| TYRP1 | ENSG00000107165.7 | P17643 | 7306 | tyrosinase-related protein 1 |
| UNC5B | ENSG00000107731.2 | Q8IZJ1 | 23663 | unc-5 homolog B (C. elegans) |
| VASN | ENSG00000168140.1 | Q6EMK4 | 114990 | vasorin |
| VDR | ENSG00000111424.8 | P11473 | 7421 | vitamin D (1,25-dihydroxyvitamin D3) receptor |
| VGLL3 | ENSG00000206538.4 | A8MV65 | 389136 | vestigial-like family member 3 |
| VSIG10L | ENSG00000186806.2 | Q86VR7 | 147645 | V-set and immunoglobulin domain containing 10 |
| | | | | like |
| WFDC12 | ENSG00000168703.1 | Q8WWY7 | 128488 | WAP four-disulfide core domain 12 |
| WNT11 | ENSG00000085741.4 | O96014 | 7481 | wingless-type MMTV integration site family, |
| | | | | member 11 |
| WNT3 | ENSG00000108379.4 | P56703 | 7473 | wingless-type MMTV integration site family, |
| | | | | member 3 |
| WNT4 | ENSG00000162552.3 | P56705 | 54361 | wingless-type MMTV integration site family, |
| | | | | member 4 |
| WNT5A | ENSG00000114251.7 | P41221 | 7474 | wingless-type MMTV integration site family, |
| | | | | member 5A |
| XG | ENSG00000124343.6 | P55808 | 7499 | Xg blood group |
| ZBTB7C | ENSG00000184828.28 | A1YPR0 | 201501 | zinc finger and BTB domain containing 7C |
| ZC3H12A | ENSG00000163874.4 | Q5D1E8 | 80149 | zinc finger CCCH-type containing 12A |
| ZNF185 | ENSG00000147394.12 | O15231 | 7739 | zinc finger protein 185 (LIM domain) |
| ZNF296 | ENSG00000170684.2 | Q8WUU4 | 49853 | zinc finger protein 296 |
| ZNF385A | ENSG00000161642.15 | Q96PM9 | 25946 | zinc finger protein 385A |
| ZNF423 | ENSG00000102935.8 | Q2M1K9 | 23090 | zinc finger protein 423 |
| ZNF521 | ENSG00000198795.14 | Q96K83 | 25925 | zinc finger protein 521 |

In one embodiment, the invention provides a set of 128 genes whose expression is up-regulated and a set of 513 genes whose expression is down-regulated for use in identifying a patient having a poor prognosis for treating early stage melanoma. In one embodiment, the invention comprises a gene expression based biomarker comprising up-regulated genes and down-regulated genes, wherein the down-regulated genes are listed in Table 2, and the up-regulated genes are listed in Table 1. In a sub-embodiment, the invention provides the identification of a gene expression based biomarker that is predictive of a patient's response to treatment. In a sub-embodiment, the invention relates to the identification of a genome wide tumor derived gene expression based biomarker that can be used in identifying, classifying, and/or treating melanoma patients with early disease.

In one embodiment, the invention provides a gene expression based biomarker comprising at least 5 genes listed in Table 1 and at least 5 genes listed in Table 2 that is correlated with the clinical need of treatment for a patient who has been diagnosed with melanoma.

In one embodiment, the invention provides a method of using a gene expression based biomarker to identify melanoma patients with a poor prognosis in early stage disease.

In one embodiment, the invention provides a method of treating a melanoma patient with early stage disease by identification of the patient with a gene expression based biomarker. In another embodiment, the invention provides a method of identifying melanoma patients who are at risk to have metastatic melanoma versus primary melanoma. In one embodiment, the invention relates to identification of a patient with an elevated level of a up-regulated gene expression based biomarker and a decreased level of down-regulated gene expression based biomarker, wherein the up-regulated gene expression based biomarker comprises 5 or more up-regulated genes from Table 1, and the down-regulated gene expression based biomarker comprises 5 or more down-regulated genes from Table 2, and wherein the patient has an elevated risk to develop metastatic melanoma, to evaluate for further treatment options.

In some embodiments, the invention relates to identifying a melanoma patient having a poor prognosis. In a sub-embodiment, the patient having a poor prognosis is likely to have a reoccurrence of melanoma, metastatic disease progress, or poor overall survival.

In some embodiments of the invention, the melanoma is early stage. In another embodiment, the melanoma is primary melanoma. In another embodiment, the melanoma is metastatic melanoma.

In some embodiments, the invention relates to classifying a patient as having a poor prognosis based on a gene expression level by calculating an elevated level of gene expression of 5 or more up-regulated genes listed in Table 1 and a lower expression of 5 or more down-regulated genes listed in Table 2. A further sub-embodiment is to classify a patient as having either a favorable prognosis or a poor prognosis based on a gene expression level by calculating a level of gene expression of 5 or more up-regulated genes listed in Table 1 and 5 or more down-regulated genes listed in Table 2, wherein a positive gene expression level of the 5 or more genes listed in Table 1 and a low expression level of the 5 or more genes listed in Table 2 indicates a patient with a pathology related to metastatic melanoma and wherein a low gene expression level of the 5 or more genes listed in Table 1 and a high expression level of the 5 or more genes listed in Table 2 indicates a patient with a pathology related to primary melanoma A positive level of gene expression of 5 or more up-regulated genes in Table 1 indicates a patient is determined to have poor prognosis and therefore in need of further treatment. A lower level of gene expression of 5 or more down-regulated genes in Table 2 indicates a patient is determined to have poor prognosis and therefore in need of further treatment.

In a sub-embodiment, the invention relates to the calculation of a positive level of gene expression used in determining a threshold for patients in a clinical trial setting. A further sub-embodiment comprises changing the threshold based on clinical outcomes designated for the clinical trial.

In one embodiment, the invention relates to selecting those melanoma patients having a poor prognosis based on having an elevated level of gene expression of 5 or more up-regulated genes listed in Table 1 and/or a lower expression level of 5 or more down-regulated genes listed in Table 2 for participation in clinical trials to evaluate the patient's response to treatment and to facilitate efficacious treatments and therapies for such patients with an unmet clinical need. The invention further relates to selecting those patients having a poor prognosis for clinical trials in order to effectively evaluate a new treatment method.

In one embodiment, the invention relates to identifying a gene expression based biomarker within a sample obtained from a patient to calculate a gene signature score.

In a further embodiment, the invention relates to calculating a gene signature score based on the up-regulated genes listed in Table 1 and down-regulated genes listed in Table 2 to determine a prognosis for a melanoma patient. The gene signature score can take into account the desire for higher expression of up-regulated genes and lower expression of down-regulated genes. In a further embodiment, the classification of a prognosis for a melanoma patient allows for treatment with an appropriate treatment option. A patient with favorable prognosis may not have a clinical need for additional treatment and can avoid possible side effects.

In one embodiment, the invention relates to the use of a gene expression based biomarker signature score for a gene expression based biomarker which comprises a set of at least about 5 of the up-regulated genes listed in Table 1 and at least 5 of the down-regulated genes from Table 2 to determine prognosis of a melanoma patient.

In particular embodiments, the gene expression based biomarker comprises at least 5 (five) genes selected from the genes listed in Table 1 at least 5 genes selected from the genes listed in Table 2 In other embodiments, the gene expression based biomarker comprises at least 5 genes, at least 6 genes, at least 7 genes, at least 8 genes, at least 9 genes, at least 10 genes, at least 11 genes, at least 12 genes, at least 13 genes, etc. or at least 128 genes from Table 1 and at least 5 genes, at least 6 genes, at least 7 genes, at least 8 genes, at least 9 genes, at least 10 genes, at least 11 genes, at least 12 genes, at least 13 genes, etc. or at least 513 genes from the genes listed in Table 2.

In one embodiment, the gene expression based biomarker comprises the following genes: ABHD10, ABHD3, ACVR2B, ADAL, ALG13, ANGEL1, ATG16L1, B4GALT3, BRAF, BRSK1, C12orf60, C1orf56, C4A, C7, CCDC151, CCDC93, CCNE1, CDID, CD38, CD5L, CDC42SE1, CHEK2, CHORDC1, CMTM7, CPOX, CR1, CRELD1, CRNKL1, CSEIL, DARS2, DBNDD2, DDIT4, DEFB108B, DHODH, DNAJB9, DNAJC5B, DPM3, DTNB, EIF4A2, ERP29, ESM1, EXOC4, FAM122B, FANCL, FMNL2, FUBP1, GGA2, GHRH, GLUL, GPN3, HBE1, HELB, HEMK1, INPP5B, KCNJ10, L3MBTL1, LHFPL1, LIPT1, MAGED1, MBOAT1, MDM1, MERTK, METTL3, METTL7B, MGAT4A, MMD, MPI, MRM1, MSH6, MSI2, MSL2, NAPB, NBPF1, NDUFAF3, NLK, NT5DC3, OLIG2, OMA1, OXNAD1, P4HA1, PDIA4, PGBD2, PHF6, PIP5KIA, PMS2, POLR3K, PREPL, RAB3GAP2, RBM39, RBM45, RNF2, RRN3, SEC24A, SFXN2, SIGLECI 1, SLC30A3, SNAPC3, SPAG4, SPIN3, SRPRB, SRSF9, STRBP, STX16, SYS1, TAF1A, TGM2, THOC2, TMEM182, TMEM81, TOP1, TP53BP1, TRIM5, TRNT1, TRPM2, UBFD1, URB2, VRK3, WDR76, WDSUB1, XPO1, ZMYND8, ZNF189, ZNF26, ZNF337, ZNF544, ZNF550, ZNF572, and ZNF841.

### III. Methods and Uses of the Invention Including Signature Score

One embodiment of the invention relates to the use of a gene expression based biomarker of the invention to evaluate or compare tumor samples obtained from a patient and predict the patient's response to cancer therapy agents, cancer progression, cancer reoccurrence, cancer prognosis and/or to determine a patient's cancer prognosis. Yet another embodiment of the invention relates to the use of mRNA whose expression levels are shown to correlate with the gene expression based biomarker to predict cancer progression, cancer reoccurrence, and cancer prognosis in a cancer patient.

In one embodiment, the invention identifies 128 up-regulated genes and 513 down-regulated genes associated with differential expression between primary and metastatic melanoma tumors. In one embodiment, the invention identifies 128 up-regulated genes and 513 down-regulated genes whose expression is correlated in melanoma patients with metastatic tumors compared to primary melanoma tumors.

In one embodiment, the invention provides a method of determining the clinical need of a patient with melanoma for a drug treatment that induces a therapeutically beneficial response in cancer cells, wherein said patient is predicted to be in clinical need of said treatment if a sample of the cancer cells is classified as having a positive level of the gene expression based biomarker defined by 5 or more genes from Table 1 or a lower expression level of the gene expression based biomarker defined by 5 or more genes from Table 2.

In another embodiment, the invention provides a method for testing a tumor for the presence or absence of a biomarker that predicts clinical need for treatment with a PD-1 antagonist, which comprises: (a) obtaining or receiving a sample from the tumor, (b) measuring the raw RNA expression level in the tumor for each gene in a gene expression based biomarker; (c) normalizing each of the measured raw RNA expression levels; (d) calculating the arithmetic mean of the normalized RNA expression levels for each of the genes to generate a score for the gene expression based biomarker; (e) classifying the tumor as biomarker positive or biomarker negative; wherein the gene expression based biomarker comprises (i) at least 5 genes selected from the genes listed in Table 1, which have a positive correlation to the signature score, (ii) at least 5 genes selected from the genes listed in Table 2 which have a negative correlation to the signature score, or (iii) a combination of at least 5 genes from Table 1 having a positive correlation to the signature score and/or the genes listed in Table 2 having a negative correlation to the signature score; and (f) classifying the tumor as biomarker positive or biomarker negative, wherein the patient is determined to have a poor prognosis if the tumor is classified as biomarker positive, and favorable survival group if the tumor is biomarker negative; and wherein a tumor is biomarker positive if the calculated score is higher than the reference score of the gene expression based biomarker and wherein a tumor is biomarker negative if the calculated score is lower than the reference score of the gene expression based biomarker.

In particular embodiments, classifying the tumor as biomarker positive or negative comprises comparing the calculated score to a reference score. In particular embodiments, step (b) comprises normalizing each of the measured raw RNA levels for each gene in the gene expression based biomarker using the measured RNA levels of a set of normalization genes.

In particular embodiments, the normalization gene set comprises 10 to 12 genes. In an embodiment of any of the above aspects of the invention, the gene expression platform comprises the 11 genes listed in Table 3 below.

TABLE 3
Normalization Gene Set

| | | Exemplary |
|---|---|---|
| Gene Symbol | Accession No. | Target Region |
| ABCF1 | NM_001090.2 | 850-950 |
| C14ORF102 | NM_017970.3 | 3236-3336 |
| G6PD | NM_000402.2 | 1155-1255 |
| OAZ1 | NM_004152.2 | 313-413 |
| POLR2A | NM_000937.2 | 3775-3875 |
| SDHA | NM_004168.1 | 230-330 |
| STK11IP | NM_052902.2 | 565-665 |
| TBC1D10B | NM_015527.3 | 2915-3015 |
| TBP | NM_001172085.1 | 587-687 |
| UBB | NM_018955.2 | 795-895 |
| ZBTB34 | NM_001099270.1 | 406-506 |

By measuring RNA levels for each gene in Table 1 and/or Table 2 and then computing signature scores from the normalized RNA levels for only the genes in each gene signature of interest, a gene expression analysis system may be used to generate and evaluate gene signature scores for different gene signatures and different tumor types.

Gene signature scores may be derived by using the entire clinical prognosis gene set (i.e. all of the genes specified in Table 1, all of the genes specified in Table 2, or all the genes specified in Tables 1 and 2, or a selection of genes from Table 1, a selection of genes from Table 2, or a selection of genes from Table 1 and Table 2), or any subset thereof, as a set of input covariates to multivariate statistical models that will determine signature scores using the fitted model coefficients, for example the linear predictor in a logistic or Cox regression. One specific example of a multivariate strategy is the use of elastic net modeling (Zou & Hastie, 2005, J. R. Statist Soc. B, 67(2). 301-320, Simon et al., 2011, J. Statistical Software 39(5): 1-13), which is a penalized regression approach that uses a hybrid between the penalties of the lasso and ridge regression, with cross-validation to select the penalty parameters. Because the RNA expression levels for most, if not all, of the clinical prognosis genes are expected to be prognostic, in one embodiment the L1 penalty parameter may be set very low, effectively running a ridge regression.

A multivariate approach may use a meta-analysis that combines data across cancer indications or may be applied within a single cancer indication. In either case, analyses would use the normalized intra-tumoral RNA expression levels of the signature gene as the input predictors, with clinical prognostic endpoint as the dependent variable. The result of such an analysis algorithmically defines the signature score for tumor samples from the patients used in the model fit, as well as for tumor samples from future patients, as a numeric combination of the multiplication co-efficients for the normalized RNA expression levels of the signature genes that is expected to be predictive of clinical outcome. The gene signature score is determined by the linear combination of the signature genes, as dictated by the final estimated values of the elastic net model coefficients at the selected values of the tuning parameters. Specifically, for a given tumor sample, the estimated coefficient for each gene is multiplied by the normalized RNA expression level of that gene in the tumor sample and then the resulting products are summed to yield the signature score for that tumor sample. Multivariate model-based strategies other than elastic net could also be used to determine a gene signature score.

An alternative to such model-based signature scores would be to use a simple averaging approach, e.g., the signature score for each tumor sample would be defined as the average of that sample's normalized RNA expression levels for those signature genes deemed to be positively associated with the poor prognosis minus the average of that sample's normalized RNA expression levels for those signature genes deemed to be negatively associated with the poor prognosis.

Also provided herein is a method for treating cancer in a patient having a tumor which comprises administering to the patient a PD-1 antagonist if the patient is positive for a gene expression based biomarker and is therefore associated with poor prognosis. Also provided herein is a method for treating cancer in a patient having a tumor which comprises administering to the patient a PD-1 antagonist if the patient has a higher expression of up-regulated genes (genes listed in Table 1), and is therefore associated with poor prognosis, and in need of additional treatments and would likely achieve a clinical benefit from treatment with a PD-1 antagonist. Further provided is a method for treating cancer in a patient having a tumor which comprises administering to the patient a PD-1 antagonist if the patient has higher expression of the up-regulated genes or lower expression of the down-regulated genes, and is therefore associated with poor prognosis and in need of additional treatment and would likely achieve a clinical benefit from treatment with a PD-1 antagonist.

### IV. Assaying Tumor Samples for Gene Signatures and Biomarkers

A gene signature score (also referred to as a positive or elevated level for the gene signature based biomarker) is determined in a sample of tumor tissue removed from a patient. A positive level for the gene signature based biomarker is determined by elevated levels for identified genes set forth in Table 1 or lower levels for identified genes set forth in Table 2. The tumor may be primary or recurrent, and may be of any type (as described above), any stage (e.g., Stage 0, I, II, III, or IV or an equivalent of other staging system), and/or histology. The patient may be of any age, gender, treatment history and/or extent and duration of remission.

The tumor sample can be obtained by a variety of procedures including, but not limited to, surgical excision, aspiration or biopsy. The tissue sample may be sectioned and assayed as a fresh specimen; alternatively, the tissue sample may be frozen for further sectioning. In some embodiments, the tissue sample is preserved by fixing and embedding in paraffin or the like.

The tumor tissue sample may be fixed by conventional methodology, with the length of fixation depending on the size of the tissue sample and the fixative used. Neutral buffered formalin, glutaraldehyde, Bouin's and paraformaldehyde are non-limiting examples of fixatives. In some embodiments, the tissue sample is fixed with formalin. In some embodiments, the fixed tissue sample is also embedded in paraffin to prepare an FFPE tissue sample.

Typically, the tissue sample is fixed and dehydrated through an ascending series of alcohols, infiltrated and embedded with paraffin or other sectioning media so that the tissue sample may be sectioned. Alternatively, the tumor tissue sample is first sectioned and then the individual sections are fixed.

In some embodiments, the gene signature score for a tumor is determined using FFPE tissue sections of about 3-4 millimeters, and preferably 4 micrometers, which are mounted and dried on a microscope slide.

Once a suitable sample of tumor tissue has been obtained, it is analyzed to quantitate the RNA expression level for each of the genes in Table 1 (or Table 2), or for a gene signature derived therefrom (e.g., any 5 or more genes from Table 1 and/or any 5 or more genes from Table 2). The use of the phrase “determine the RNA expression level of a gene” or “determine the RNA level” of each gene as used herein refers to detecting and quantifying RNA transcribed from that gene. The term “RNA transcript” includes mRNA transcribed from the gene, and/or specific spliced variants thereof and/or fragments of such mRNA and spliced variants.

A person skilled in the art will appreciate that a number of methods can be used to isolate RNA from the tissue sample for analysis. For example, RNA may be isolated from frozen tissue samples by homogenization in guanidinium isothiocyanate and acid phenol-chloroform extraction. Commercial kits are available for isolating RNA from FFPE samples. If the tumor sample is an FFPE tissue section on a glass slide, it is possible to perform gene expression analysis on whole cell lysates rather than on isolated total RNA.

Persons skilled in the art are also aware of several methods useful for detecting and quantifying the level of RNA transcripts within the isolated RNA or whole cell lysates. Quantitative detection methods include, but are not limited to, arrays (i.e., microarrays), quantitative real time PCR (RT-PCR), multiplex assays, nuclease protection assays, and Northern blot analyses. Generally, such methods employ labeled probes that are complimentary to a portion of each transcript to be detected. Probes for use in these methods can be readily designed based on the known sequences of the genes and the transcripts expressed thereby. Suitable labels for the probes are well-known and include, e.g., fluorescent, chemiluminescent and radioactive labels.

In some embodiments, assaying a tumor sample for expression of the genes in Table 1, or gene signatures derived therefrom (i.e. gene signatures comprising 5 or more genes from Table 1, or likewise with Table 2), employs detection and quantification of RNA levels in real-time using nucleic acid sequence based amplification (NASBA) combined with molecular beacon detection molecules. NASBA is described, e.g., in Compton, Nature 350 (6313):91-92 (1991). NASBA is a single-step isothermal RNA-specific amplification method. Generally, the method involves the following steps: RNA template is provided to a reaction mixture, where the first primer attaches to its complementary site at the 3′ end of the template; reverse transcriptase synthesizes the opposite, complementary DNA strand; RNAse H destroys the RNA template (RNAse H only destroys RNA in RNA-DNA hybrids, but not single-stranded RNA); the second primer attaches to the 3′ end of the DNA strand, and reverse transcriptase synthesizes the second strand of DNA; and T7 RNA polymerase binds double-stranded DNA and produces a complementary RNA strand which can be used again in step 1, such that the reaction is cyclic.

In other embodiments, the assay format is a flap endonuclease-based format, such as the Invader™ assay (Third Wave Technologies). In the case of using the invader method, an invader probe containing a sequence specific to the region 3′ to a target site, and a primary probe containing a sequence specific to the region 5′ to the target site of a template and an unrelated flap sequence, are prepared. Cleavase is then allowed to act in the presence of these probes, the target molecule, as well as a FRET probe containing a sequence complementary to the flap sequence and an auto-complementary sequence that is labeled with both a fluorescent dye and a quencher. When the primary probe hybridizes with the template, the 3′ end of the invader probe penetrates the target site, and this structure is cleaved by the Cleavase resulting in dissociation of the flap. The flap binds to the FRET probe and the fluorescent dye portion's cleaved by the Cleavase resulting in emission of fluorescence.

In yet other embodiments, the assay format employs direct mRNA capture with branched DNA (QuantiGene™, Panomics) or Hybrid Capture™ (Digene).

One example of an array technology suitable for use in measuring expression of the genes in gene expression platform of the invention is the ArrayPlate™ assay technology sold by HTG Molecular, Tucson Arizona, and described in Martel, R. R., et al., Assay and Drug Development Technologies 1(1):61-71, 2002. In brief, this technology combines a nuclease protection assay with array detection. Cells in microplate wells are subjected to a nuclease protection assay. Cells are lysed in the presence of probes that bind targeted mRNA species. Upon addition of SI nuclease, excess probes and unhybridized mRNA are degraded, so that only mRNA:probe duplexes remain. Alkaline hydrolysis destroys the mRNA component of the duplexes, leaving probes intact. After the addition of a neutralization solution, the contents of the processed cell culture plate are transferred to another ArrayPlate™ called a programmed ArrayPlate™. ArrayPlates™ contain a 16-element array at the bottom of each well. Each array element comprises a position-specific anchor oligonucleotide that remains the same from one assay to the next. The binding specificity of each of the 16 anchors is modified with an oligonucleotide, called a programming linker oligonucleotide, which is complementary at one end to an anchor and at the other end to a nuclease protection probe. During a hybridization reaction, probes transferred from the culture plate are captured by immobilized programming linker. Captured probes are labeled by hybridization with a detection linker oligonucleotide, which is in turn labeled with a detection conjugate that incorporates peroxidase. The enzyme is supplied with a chemiluminescent substrate, and the enzyme-produced light is captured in a digital image. Light intensity at an array element is a measure of the amount of corresponding target mRNA present in the original cells.

In one embodiment, an array of oligonucleotides may be synthesized on a solid support. Exemplary solid supports include glass, plastics, polymers, metals, metalloids, ceramics, organics, etc. Using chip masking technologies and photoprotective chemistry, it is possible to generate ordered arrays of nucleic acid probes. These arrays, which are known, for example, as “DNA chip” or very large scale immobilized polymer arrays “VLSIPS” arrays), may include millions of defined probe regions on a substrate having an area of about 1 cm² to several cm², thereby incorporating from a few to millions of probes (see, e.g., U.S. Pat. No. 5,631,734).

To compare expression levels, labeled nucleic acids may be contacted with the array under conditions sufficient for binding between the target nucleic acid and the probe on the array. In one embodiment, the hybridization conditions may be selected to provide for the desired level of hybridization specificity; that is, conditions sufficient for hybridization to occur between the labeled nucleic acids and probes on the microarray.

Hybridization may be carried out in conditions permitting essentially specific hybridization. The length and GC content of the nucleic acid will determine the thermal melting point and thus, the hybridization conditions necessary for obtaining specific hybridization of the probe to the target nucleic acid. These factors are well known to a person of skill in the art, and may also be tested in assays. An extensive guide to nucleic acid hybridization may be found in Tijssen, et al. (Laboratory Techniques in Biochemistry and Molecular Biology, Vol. 24: Hybridization With Nucleic Acid Probes, P. Tijssen, ed.; Elsevier, N.Y. (1993)). The methods described above will result in the production of hybridization patterns of labeled target nucleic acids on the array surface. The resultant hybridization patterns of labeled nucleic acids may be visualized or detected in a variety of ways, with the particular manner of detection selected based on the particular label of the target nucleic acid. Representative detection means include scintillation counting, autoradiography, fluorescence measurement, calorimetric measurement, light emission measurement, light scattering, and the like.

One such method of detection utilizes an array scanner that is commercially available (Affymetrix, Santa Clara, Calif.), for example, the 417® Arrayer, the 418® Array Scanner, or the Agilent Gene Array® Scanner. This scanner is controlled from a system computer with an interface and easy-to-use software tools. The output may be directly imported into or directly read by a variety of software applications. Exemplary scanning devices are described in, for example, U.S. Pat. Nos. 5,143,854 and 5,424,186.

One assay method to measure transcript abundance for the genes listed in Table 1 utilizes the nCounter® Analysis System marketed by NanoString® Technologies (Seattle, Washington USA). This system, which is described by Geiss et al., Nature Biotechnol. 2(3):317-325 (2008), utilizes a pair of probes, namely, a capture probe and a reporter probe, each comprising a 35- to 50-base sequence complementary to the transcript to be detected. The capture probe additionally includes a short common sequence coupled to an immobilization tag, e.g., an affinity tag that allows the complex to be immobilized for data collection. The reporter probe additionally includes a detectable signal or label, e.g., is coupled to a color-coded tag. Following hybridization, excess probes are removed from the sample, and hybridized probe/target complexes are aligned and immobilized via the affinity or other tag in a cartridge. The samples are then analyzed, for example using a digital analyzer or other processor adapted for this purpose. Generally, the color-coded tag on each transcript is counted and tabulated for each target transcript to yield the expression level of each transcript in the sample. This system allows measuring the expression of hundreds of unique gene transcripts in a single multiplex assay using capture and reporter probes designed by NanoString.

### V. Methods of Treatment of the Invention and PD-1 Antagonists Useful in Said Methods

In some embodiments, the invention provides a gene expression based biomarker whose expression is correlated with identifying a patient who is most likely to be in need of additional treatments and as a result, to achieve a clinical benefit from treatment with a PD-1 antagonist.

This invention supports the use of such gene expression based biomarker in a variety of research and commercial applications, including but not limited to, clinical trials of PD-1 antagonists in which patients are selected on the basis of whether they test positive or negative for a gene signature based biomarker, diagnostic methods and products for determining a patient's gene signature score or for classifying a patient as positive or negative for a gene signature based biomarker, personalized treatment methods which involve altering or stopping a patient's drug therapy based on the patient's gene signature score or biomarker status, as well as pharmaceutical compositions and drug products comprising a PD-1 antagonist for use in treating patients who test positive for a gene signature biomarker.

The utility of any of the research and commercial applications claimed herein does not require that 100% of the patients who test positive for a gene signature based biomarker achieve a benefit from an anti-tumor response to a PD-1 antagonist; nor does it require a diagnostic method or kit to have a specific degree of specificity or sensitivity in determining the presence or absence of a biomarker in every patient, nor does it require that a diagnostic method claimed herein be 100% accurate in determining whether every patient is likely to have a beneficial response to a PD-1 antagonist. Thus, it is intended that the terms “determine”, “determining” and “predicting” should not be interpreted as requiring a definite or certain result; instead these terms should be construed as meaning either that a claimed method provides an accurate result for at least the majority of patients or that the result or prediction for any givzen patient is more likely to be correct than incorrect.

Preferably, the accuracy of the result provided by a diagnostic method of the invention is one that a skilled artisan or regulatory authority would consider suitable for the particular application in which the method is used. Similarly, the utility of the claimed drug products and treatment methods does not require that the claimed or desired effect is produced in every cancer patient; all that is required is that a clinical practitioner, when applying his or her professional judgment consistent with all applicable norms, decides that the chance of achieving the claimed effect of treating a given patient according to the claimed method or with the claimed composition or drug product.

In one aspect, the invention relates to a method for testing a tumor for the presence or absence of a biomarker that predicts patient clinical need for additional treatment, which comprises: (a) obtaining or receiving a sample from the tumor, (b) measuring the raw RNA expression level in the tumor sample for each gene in a melanoma gene signature; (c) normalizing each of the measured raw RNA expression levels; and (d) calculating the arithmetic mean of the normalized RNA expression levels for each of the genes to generate a score for the gene expression based biomarker; wherein the gene expression based biomarker comprises at least 5 out of 128 genes selected from the genes listed in Table 1: (e) comparing the calculated score to a reference score for the melanoma gene signature; and (f) classifying the tumor as biomarker positive or biomarker negative; wherein if the calculated score is equal to or less than the reference score, then the tumor is classified as biomarker positive, and if the biomarker signature score is greater than the reference gene expression based biomarker signature score, then the tumor is classified as biomarker negative.

In one aspect, the invention relates to a method for testing a tumor for the presence or absence of a biomarker that predicts clinical need for additional treatment, which comprises: (a) obtaining or receiving a sample from the tumor, (b) measuring the raw RNA expression level in the tumor sample for each gene in a melanoma gene signature; (c) normalizing each of the measured raw RNA expression levels; and (d) calculating the arithmetic mean of the normalized RNA expression levels for each of the genes to generate a score for the gene expression based biomarker; wherein the gene expression based biomarker comprises at least 5 out of 513 genes selected from the genes listed in Table 2; (e) comparing the calculated score to a reference score for the melanoma gene signature; and (f) classifying the tumor as biomarker positive or biomarker negative; wherein if the calculated score is equal to or less than the reference score, then the tumor is classified as biomarker positive, and if the biomarker signature score is greater than the reference gene expression based biomarker signature score, then the tumor is classified as biomarker negative.

The invention provides methods of treating cancer in a human patient comprising administering to the patient a PD-1 antagonist, wherein the patient has tested positive for a gene expression based biomarker (i.e., the patient has a tumor which has a calculated gene signature score from a gene signature comprised of 5 or more genes from Table 1 that is equal to or greater than a reference score, or the patient has a tumor which has a calculated gene signature score from a gene signature comprised of 5 or more genes from Table 2 that is equal to or less than a reference score). PD-1 antagonists useful in the treatment methods of the invention include anti-PD-1 antibodies, or antigen binding fragments thereof, that specifically bind to PD-1 and block binding of PD-1 to PD-L1 and/or PD-L2. Other PD-1 antagonists useful in the treatment methods of the invention include anti-PD-L1 antibodies, or antigen binding fragments thereof, that specifically bind to PD-L1 and block binding of PD-L1 to PD-1.

In particular embodiments, the PD-1 antagonist is an anti-PD-1 antibody, or antigen binding fragment thereof. In alternative embodiments, the PD-1 antagonist is an anti-PD-L1 antibody, or antigen binding fragment thereof. In some embodiments, the PD-1 antagonist is pembrolizumab (KEYTRUDA™, Merck Sharp & Dohme LILC, Rahway, NJ, USA), nivolumab (OPDIVOT™, Bristol-Myers Squibb Company, Princeton, NJ, USA), atezolizumab (TECENTRIQ™, Genentech, San Francisco, CA, USA), durvalumab (IMFINZIM, AstraZeneca Pharmaceuticals LP, Wilmington, DE), cemiplimab (LIBTAYO™, Regeneron Pharmaceuticals, Tarrytown, NY, USA) avelumab (BAVENCIO™, Merck KGaA, Darmstadt, Germany) or dostarlimab (JEMPERLI™, GlaxoSmithKline LLC, Philadelphia, PA). In other embodiments, the PD-1 antagonist is pidilizumab (U.S. Pat. No. 7,332,582), AMP-514 (MedImmune LLC, Gaithersburg, MD, USA), PDR001 (U.S. Pat. No. 9,683,048), BGB-A317 (U.S. Pat. No. 8,735,553), or MGA012 (MacroGenics, Rockville, MD).

In some embodiments, the PD-1 antagonist is an anti-human PD-1 antibody, antigen binding fragment thereof, or variant thereof disclosed in any of U.S. Pat. Nos. 7,488,802, 7,521,051, 8,008,449, 8,354,509, 8,168,757, WO2004/004771, WO2004/072286, WO2004/056875, US2011/0271358, and WO 2008/156712, the disclosures of which are incorporated by reference herein in their entireties.

In some embodiments, the PD-1 antagonist is pembrolizumab. In particular sub-embodiments, the method comprises administering 200 mg of pembrolizumab to the patient about every three weeks. In other sub-embodiments, the method comprises administering 400 mg of pembrolizumab to the patient about every six weeks.

In further sub-embodiments, the method comprises administering 2 mg/kg of pembrolizumab to the patient about every three weeks. In particular sub-embodiments, the patient is a pediatric patient.

In some embodiments, the PD-1 antagonist is nivolumab. In particular sub-embodiments, the method comprises administering 240 mg of nivolumab to the patient about every two weeks. In other sub-embodiments, the method comprises administering 360 mg of nivolumab to the patient about every three weeks. In other sub-embodiments, the method comprises administering 480 mg of nivolumab to the patient about every four weeks.

In some embodiments, the PD-1 antagonist is atezolizumab. In particular sub-embodiments, the method comprises administering 1200 mg of atezolizumab to the patient about every three weeks.

In some embodiments, the PD-1 antagonist is durvalumab. In particular sub-embodiments, the method comprises administering 10 mg/kg of durvalumab to the patient about every two weeks.

In some embodiments, the PD-1 antagonist is cemiplimab. In particular embodiments, the method comprises administering 350 mg of cemiplimab to the patient about every three weeks.

In some embodiments, the PD-1 antagonist is avelumab. In particular sub-embodiments, the method comprises administering 800 mg of avelumab to the patient about every two weeks.

Table 4 provides amino acid sequences for exemplary anti-human PD-1 antibodies pembrolizumab and nivolumab. Alternative PD-1 antibodies and antigen-binding fragments that are useful in the formulations and methods of the invention are shown in Table 5.

In some embodiments of the methods of treatment of the invention, a PD-1 antagonist is an anti-human PD-1 antibody or antigen binding fragment thereof or an anti-human PD-L1 antibody or antigen binding fragment thereof, which comprises three light chain CDRs of CDRL1, CDRL2 and CDRL3 and/or three heavy chain CDRs of CDRH1, CDRH2 and CDRH3.

In one embodiment of the methods of treatment of the invention, CDRL1 is SEQ ID NO:1 or a variant of SEQ ID NO:1, CDRL2 is SEQ ID NO:2 or a variant of SEQ ID NO:2, and CDRL3 is SEQ ID NO:3 or a variant of SEQ ID NO:3.

In one embodiment, CDRH1 is SEQ ID NO:6 or a variant of SEQ ID NO:6, CDRH2 is SEQ ID NO: 7 or a variant of SEQ ID NO:7, and CDRH3 is SEQ ID NO:8 or a variant of SEQ ID NO:8.

In one embodiment, the three light chain CDRs are SEQ ID NO:1, SEQ ID NO:2, and SEQ ID NO:3 and the three heavy chain CDRs are SEQ ID NO:6, SEQ ID NO:7 and SEQ ID NO:8.

In an alternative embodiment of the invention, CDRL1 is SEQ ID NO:11 or a variant of SEQ ID NO:11, CDRL2 is SEQ ID NO:12 or a variant of SEQ ID NO:12, and CDRL3 is SEQ ID NO:13 or a variant of SEQ ID NO: 13.

In one embodiment, CDRH1 is SEQ ID NO:16 or a variant of SEQ ID NO:16, CDRH2 is SEQ ID NO:17 or a variant of SEQ ID NO:17, and CDRH3 is SEQ ID NO:18 or a variant of SEQ ID NO:18.

In one embodiment, the three light chain CDRs are SEQ ID NO:1, SEQ ID NO:2, and SEQ ID NO:3 and the three heavy chain CDRs are SEQ ID NO:6, SEQ ID NO:7 and SEQ ID NO:8.

In an alternative embodiment, the three light chain CDRs are SEQ ID NO:11, SEQ ID NO:12, and SEQ ID NO:13 and the three heavy chain CDRs are SEQ ID NO:16. SEQ ID NO 17 and SEQ ID NO: 18.

In a further embodiment of the invention, CDRL1 is SEQ ID NO:21 or a variant of SEQ ID NO:21, CDRL2 is SEQ ID NO:22 or a variant of SEQ ID NO:22, and CDRL3 is SEQ ID NO:23 or a variant of SEQ ID NO:23.

In yet another embodiment, CDRH1 is SEQ ID NO:24 or a variant of SEQ ID NO:24, CDRH2 is SEQ ID NO: 25 or a variant of SEQ ID NO:25, and CDRH3 is SEQ ID NO:26 or a variant of SEQ ID NO:26.

In another embodiment, the three light chain CDRs are SEQ ID NO:21, SEQ ID NO:22, and SEQ ID NO:23 and the three heavy chain CDRs are SEQ ID NO:24, SEQ ID NO:25 and SEQ ID NO:26.

Some antibody and antigen binding fragments of the methods of treatment of the invention comprise a light chain variable region and a heavy chain variable region. In some embodiments, the light chain variable region comprises SEQ ID NO:4 or a variant of SEQ ID NO:4, and the heavy chain variable region comprises SEQ ID NO:9 or a variant of SEQ ID NO:9. In further embodiments, the light chain variable region comprises SEQ ID NO:14 or a variant of SEQ ID NO:14, and the heavy chain variable region comprises SEQ ID NO:19 or a variant of SEQ ID NO:19. In further embodiments, the heavy chain variable region comprises SEQ ID NO:27 or a variant of SEQ ID NO:27 and the light chain variable region comprises SEQ ID NO:28 or a variant of SEQ ID NO:28, SEQ ID NO:29 or a variant of SEQ ID NO:29, or SEQ ID NO:30 or a variant of SEQ ID NO:30. In such embodiments, a variant light chain or heavy chain variable region sequence is identical to the reference sequence except having one, two, three, four or five amino acid substitutions. In some embodiments, the substitutions are in the framework region (i.e., outside of the CDRs). In some embodiments, one, two, three, four or five of the amino acid substitutions are conservative substitutions.

In one embodiment of the methods of treatment of the invention, the PD-1 antagonist is an antibody or antigen binding fragment that comprises a light chain variable region comprising or consisting of SEQ ID NO:4 and a heavy chain variable region comprising or consisting SEQ ID NO:9. In a further embodiment, the antibody or antigen binding fragment comprises a light chain variable region comprising or consisting of SEQ ID NO 14 and a heavy chain variable region comprising or consisting of SEQ ID NO:19. In one embodiment of the methods of the invention, the antibody or antigen binding fragment comprises a light chain variable region comprising or consisting of SEQ ID NO:28 and a heavy chain variable region comprising or consisting SEQ ID NO:27. In a further embodiment, the antibody or antigen binding fragment comprises a light chain variable region comprising or consisting of SEQ ID NO:29 and a heavy chain variable region comprising or consisting SEQ ID NO:27. In another embodiment, the antibody or antigen binding fragment comprises a light chain variable region comprising or consisting of SEQ ID NO:30 and a heavy chain variable region comprising or consisting SEQ ID NO:27.

In another embodiment of the methods of treatment of the invention, the PD-1 antagonist is an antibody or antigen binding protein that has a V_{L} domain and/or a V_{H} domain with at least 95%, 90%, 85%, 80%, 75% or 50% sequence homology to one of the V_{L} domains or V_{H} domains described above, and exhibits specific binding to PD-1. In another embodiment of the methods of treatment of the invention, the PD-1 antagonist is an antibody or antigen binding protein comprising V_{L} and V_{H} domains having up to 1, 2, 3, 4, or 5 or more amino acid substitutions, and exhibits specific binding to PD-1.

In any of the embodiments above, the PD-1 antagonist may be a full-length anti-PD-1 antibody or an antigen binding fragment thereof that specifically binds human PD-1, or a full-length anti-PD-L1 antibody or an antigen binding fragment thereof that specifically binds human PD-L1. In certain embodiments, the anti-PD-1 antibody or anti-PD-L1 antibody is selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄ Different constant domains may be appended to the V_{L} and V_{H} regions provided herein. For example, if a particular intended use of an antibody (or fragment) of the invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances an IgG4 constant domain, for example, may be used.

In embodiments of the methods of treatment of the invention, the PD-1 antagonist is an anti-PD-1 antibody comprising a light chain comprising or consisting of a sequence of amino acid residues as set forth in SEQ ID NO:5 and a heavy chain comprising or consisting of a sequence of amino acid residues as set forth in SEQ ID NO 0. In alternative embodiments, the PD-1 antagonist is an anti-PD-1 antibody comprising a light chain comprising or consisting of a sequence of amino acid residues as set forth in SEQ ID NO:15 and a heavy chain comprising or consisting of a sequence of amino acid residues as set forth in SEQ ID NO:20. In further embodiments, the PD-1 antagonist is an anti-PD-1 antibody comprising a light chain comprising or consisting of a sequence of amino acid residues as set forth in SEQ ID NO:32 and a heavy chain comprising or consisting of a sequence of amino acid residues as set forth in SEQ ID NO:31. In additional embodiments, the PD-1 antagonist is an anti-PD-1 antibody comprising a light chain comprising or consisting of a sequence of amino acid residues as set forth in SEQ ID NO:33 and a heavy chain comprising or consisting of a sequence of amino acid residues as set forth in SEQ ID NO:31. In yet additional embodiments, the PD-1 antagonist is an anti-PD-1 antibody comprising a light chain comprising or consisting of a sequence of amino acid residues as set forth in SEQ ID NO:34 and a heavy chain comprising or consisting of a sequence of amino acid residues as set forth in SEQ ID NO:31.

In some embodiments of the methods of treatment of the invention, the PD-1 antagonist is pembrolizumab, a pembrolizumab variant or a pembrolizumab biosimilar. In some embodiments, the PD-1 antagonist is nivolumab, a nivolumab variant or a nivolumab biosimilar. In some embodiments, the PD-1 antagonist is atezolizumab, an atezolizumab variant or an atezolizumab biosimilar. In some embodiments, the PD-1 antagonist is durvalumab, a durvalumab variant or a durvalumab biosimilar. In some embodiments, the PD-1 antagonist is cemiplimab, a cemiplimab variant or a cemiplimab biosimilar. In some embodiments, the PD-1 antagonist is avelumab, an avelumab variant or an avelumab biosimilar. In some embodiments, the PD-1 antagonist is dostarlimab, a dostarlimab variant or a dostarlimab biosimilar.

Ordinarily, amino acid sequence variants of the PD-1 antagonists useful in the methods of treatment of the invention will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%/o, and most preferably at least 95, 96, 97, 98, or 99% identity. Identity or homology with respect to a sequence is defined herein as the percentage of amino acid residues in the candidate sequence that are identical with the anti-PD-1 residues, after aligning the sequences and introducing gaps, if necessary (including gaps at either end of the sequence, or truncations), to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410: Gish, W., et al., (1993) Nature Genet. 3:266-272; Madden, T. L., et al., (1996) Meth. Enzymol. 266:131-141; Altschul, S F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS; Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3. “M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 366-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Nat. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

Likewise, either class of light chain can be used in the compositions and methods herein. Specifically, kappa, lambda, or variants thereof are useful in the present compositions and methods.

TABLE 4
Exemplary Anti-PD-1 Antibody Sequences

| | | |
|---|---|---|
| | | SEQ |
| Antibody | | ID |
| Feature | Amino Acid Sequence | NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERA | 4 |
| Region | TLSCRASKGVSTSGYSYLH | |
| | WYQQKPGQAPRLLIYLASY | |
| | LESGVPARFSGSGSGTDFT | |
| | LTISSLEPEDFAVYYCQHS | |
| | RDLPLTFGGGTKVEIK | |
| Light | EIVLTQSPATLSLSPGERA | 5 |
| Chain | TLSCRASKGVSTSGYSYLH | |
| | WYQQKPGQAPRLLIYLASY | |
| | LESGVPARFSGSGSGTDFT | |
| | LTISSLEPEDFAVYYCQHS | |
| | RDLPLTFGGGTKVEIKRTV | |
| | AAPSVFIFPPSDEQLKSGT | |
| | ASVVCLLNNFYPREAKVQW | |
| | KVDNALQSGNSQESVTEQD | |
| | SKDSTYSLSSTLTLSKADY | |
| | EKHKVYACEVTHQGLSSPV | |
| | TKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGASVK | 9 |
| Region | VSCKASGYTFTNYYMYWVR | |
| | QAPGQGLEWMGGINPSNGG | |
| | TNFNEKFKNRVTLTTDSST | |
| | TTAYMELKSLQFDDTAVYY | |
| | CARRDYRFDMGFDYWGQGT | |
| | TVTVSS | |
| Heavy | QVQLVQSGVEVKKPGASVK | 10 |
| Chain | VSCKASGYTFTNYYMYWVR | |
| | QAPGQGLEWMGGINPSNGG | |
| | TNFNEKFKNRVTLTTDSST | |
| | TTAYMELKSLQFDDTAVYY | |
| | CARRDYRFDMGFDYWGQGT | |
| | TVTVSSASTKGPSVFPLAP | |
| | CSRSTSESTAALGCLVKDY | |
| | FPEPVTVSWNSGALTSGVH | |
| | TFPAVLQSSGLYSLSSVVT | |
| | VPSSSLGTKTYTCNVDHKP | |
| | SNTKVDKRVESKYGPPCPP | |
| | CPAPEFLGGPSVFLFPPKP | |
| | KDTLMISRTPEVTCVVVDV | |
| | SQEDPEVQFNWYVDGVEVH | |
| | NAKTKPREEQFNSTYRVVS | |
| | VLTVLHQDWLNGKEYKCKV | |
| | SNKGLPSSIEKTISKAKGQ | |
| | PREPQVYTLPPSQEEMTKN | |
| | QVSLTCLVKGFYPSDIAVE | |
| | WESNGQPENNYKTTPPVLD | |
| | SDGSFFLYSRLTVDKSRWQ | |
| | EGNVFSCSVMHEALHNHYT | |
| | QKSLSLSLGK | |

Nivolumab Light Chain

| | | |
|---|---|---|
| CDR1 | RASQSVSSYLA | 11 |
| CDR2 | DASNRAT | 12 |
| CDR3 | QQSSNWPRT | 13 |
| Variable | EIVLTQSPATLSLSPGERA | 14 |
| Region | TLSCRASQSVSSYLAWYQQ | |
| | KPGQAPRLLIYDASNRATG | |
| | IPARFSGSGSGTDFTLTIS | |
| | SLEPEDFAVYYCQQSSNWP | |
| | RTFGQGTKVEIK | |
| Light | EIVLTQSPATLSLSPGERA | 15 |
| Chain | TLSCRASQSVSSYLAWYQQ | |
| | KPGQAPRLLIYDASNRATG | |
| | IPARFSGSGSGTDFTLTIS | |
| | SLEPEDFAVYYCQQSSNWP | |
| | RTFGQGTKVEIKRTVAAPS | |
| | VFIFPPSDEQLKSGTASVV | |
| | CLLNNFYPREAKVQWKVDN | |
| | ALQSGNSQESVTEQDSKDS | |
| | TYSLSSTLTLSKADYEKHK | |
| | VYACEVTHQGLSSPVTKSF | |
| | NRGEC | |

Nivolumab Heavy Chain

| | | |
|---|---|---|
| CDR1 | NSGMH | 16 |
| CDR2 | VIWYDGSKRYYADSVKG | 17 |
| CDR3 | NDDY | 18 |
| Variable | QVQLVESGGGVVQPGRSLR | 19 |
| Region | LDCKASGITFSNSGMHWVR | |
| | QAPGKGLEWVAVIWYDGSK | |
| | RYYADSVKGRFTISRDNSK | |
| | NTLFLQMNSLRAEDTAVYY | |
| | CATNDDYWGQGTLVTVSS | |
| Heavy | QVQLVESGGGVVQPGRSLR | 20 |
| Chain | LDCKASGITFSNSGMHWVR | |
| | QAPGKGLEWVAVIWYDGSK | |
| | RYYADSVKGRFTISRDNSK | |
| | NTLFLQMNSLRAEDTAVYY | |
| | CATNDDYWGQGTLVTVSSA | |
| | STKGPSVFPLAPCSRSTSE | |
| | STAALGCLVKDYFPEPVTV | |
| | SWNSGALTSGVHTFPAVLQ | |
| | SSGLYSLSSVVTVPSSSLG | |
| | TKTYTCNVDHKPSNTKVDK | |
| | RVESKYGPPCPPCPAPEFL | |
| | GGPSVFLFPPKPKDTLMIS | |
| | RTPEVTCVVVDVSQEDPEV | |
| | QFNWYVDGVEVHNAKTKPR | |
| | EEQFNSTYRVVSVLTVLHQ | |
| | DWLNGKEYKCKVSNKGLPS | |
| | SIEKTISKAKGQPREPQVY | |
| | TLPPSQEEMTKNQVSLTCL | |
| | VKGFYPSDIAVEWESNGQP | |
| | ENNYKTTPPVLDSDGSFFL | |
| | YSRLTVDKSRWQEGNVFSC | |
| | SVMHEALHNHYTQKSLSLS | |
| | LGK | |

TABLE 5
Additional PD-1 Antibodies and Antigen Binding Fragments
Useful in the Methods of Treatment of the Invention.
A. Antibodies and antigen binding fragments comprising
light and heavy chain CDRs of hPD-1.08A in WO2008/156712

| | |
|---|---|
| CDRL1 | SEQ ID NO: 21 |
| CDRL2 | SEQ ID NO: 22 |
| CDRL3 | SEQ ID NO: 23 |
| CDRH1 | SEQ ID NO: 24 |
| CDRH2 | SEQ ID NO: 25 |
| CDRH3 | SEQ ID NO: 26 |

C. Antibodies and antigen binding fragments comprising
the mature h109A heavy chain variable region and one of
the mature K09A light chain variable regions in WO 2008/156712

| | |
|---|---|
| Heavy chain VR | SEQ ID NO: 27 |
| Light chain VR | SEQ ID NO: 28, SEQ ID |
| | NO: 29, SEQ ID NO: 30 |

D. Antibodies and antigen binding fragments
comprising the mature 409 heavy chain and one of the
mature K09A light chains in WO 2008/156712

| | |
|---|---|
| Heavy chain | SEQ ID NO: 31 |
| Light chain | SEQ ID NO: 32, SEQ ID |
| | NO: 33, SEQ ID NO: 34 |

In the methods of treatment of the invention, any PD-1 antagonist may be used, including for example, the PD-1 antagonists disclosed in this section.

In one embodiment, the invention provides a method for treating cancer in a patient having a tumor which comprises administering to the patient a PD-1 antagonist if the tumor is positive for a gene expression based biomarker, or administering to the patient a cancer treatment that does not include a PD-1 antagonist if the tumor is negative for the biomarker; wherein the determination of whether the tumor is positive or negative for the gene expression based biomarker was made using a method as described herein.

In one embodiment, the invention provides a method for treating cancer in a patient having a tumor, the method comprising:
(a) determining if the tumor is positive or negative for a gene expression based biomarker, wherein the determining step comprises:
   (i) obtaining a sample from the patient's tumor;
   (ii) sending the tumor sample to a laboratory with a request to test the sample for the presence or absence of the gene expression based biomarker, and
   (iii) receiving a report from the laboratory that states whether the tumor sample is biomarker positive or biomarker negative, wherein the tumor sample is classified as biomarker positive or biomarker negative using a method according to any of the methods described herein; and
(b) administering to the patient a PD-1 antagonist if the tumor is positive for the biomarker, or administering to the patient a cancer treatment that does not include a PD-1 antagonist if the tumor is negative for the biomarker.

In another embodiment, the invention provides a method for treating cancer in a patient having a tumor which comprises:
(a) determining if the tumor is positive or negative for a gene expression based biomarker, wherein the determining step comprises:
   (i) obtaining a sample from the patient's tumor;
   (ii) sending the tumor sample to a laboratory with a request to generate a gene expression based biomarker signature score;
   (iii) receiving a report from the laboratory that states the gene expression based biomarker signature score, wherein the gene expression based biomarker signature score is generated by a method comprising:
      (1) measuring the raw RNA expression level in the tumor sample for each gene in a gene expression based biomarker;
      (2) normalizing each of the measured raw RNA expression levels; and
      (3) calculating the arithmetic mean of the normalized RNA expression levels for each of the genes to generate the score for the gene expression based biomarker;
   (iv) comparing the calculated score to a reference score for the gene expression based biomarker; and
   (v) classifying the tumor as biomarker positive or biomarker negative; wherein the gene expression based biomarker comprises
   (i) at least 5 genes selected from the genes listed in Table 1 which have a positive correlation to the signature score,
   (ii) at least 5 genes listed from the genes listed in Table 2 which have a negative correlation to the signature score, or
   (iii) a combination of at least 5 genes selected from the genes listed in Table 1 having a positive correlation to the signature score and/or the genes listed in Table 2 having a negative correlation to the signature score;
wherein a tumor is biomarker positive if the calculated score is higher than the reference score of the gene expression based biomarker, and
wherein a tumor is biomarker negative if the calculated score is lower than the reference score of the gene expression based biomarker, and
wherein a biomarker positive tumor indicates a need for further treatment with a PD-1 antagonist and biomarker negative if the tumor does not indicated a need for further treatment with a PD-1 antagonist.

In particular embodiments of the method above, step (a)(iii)(2) comprises normalizing each of the measured raw RNA levels for each gene in the gene expression based biomarker signature using the measured RNA levels of a set of normalization genes.

The invention further provides a method for treating cancer in a patient having a tumor, the method comprising:
(a) determining or having determined if the tumor is positive or negative for a gene signature based biomarker;
which step comprises:
   (i) measuring the raw RNA expression level in the tumor sample for each gene in the gene signature, wherein the gene signature based biomarker comprises 5 or more genes selected from the genes listed in Table 1;
   (ii) normalizing each of the measured raw RNA expression levels;
   (iii) calculating the arithmetic mean of the normalized RNA expression levels for each of the genes to generate a score for the gene signature based biomarker; and
   (iv) classifying the tumor as biomarker positive or biomarker negative, wherein the tumor is biomarker positive if the gene signature score is greater than a predetermined threshold signature score; and
(b) administering to the patient a PD-1 antagonist if the tumor is positive for the gene expression based biomarker, or administering to the patient a cancer treatment that does not include a PD-1 antagonist if the tumor is negative for the gene expression based biomarker.

In specific embodiments of any of the methods of treatment disclosed herein, the PD-1 antagonist is pembrolizumab, nivolumab, atezolizumab, durvalumab, cemiplimab, avelumab or dostarlimab.

In one embodiment, the PD-1 antagonist is pembrolizumab or a variant of pembrolizumab.

In one embodiment, the PD-1 antagonist is nivolumab or a variant of nivolumab.

In one embodiment, the PD-1 antagonist is avelumab or a variant of avelumab.

In one embodiment, the PD-1 antagonist is durvalumab or a variant of durvalumab.

In one embodiment, the PD-1 antagonist is cemiplimab or a variant of cemiplimab.

In one embodiment, the PD-1 antagonist is atezolizumab or a variant of atezolizumab.

In one embodiment, the PD-1 antagonist is dostarlimab or a variant of dostarlimab.

The methods of treatment of the invention may be useful for treating cancer, wherein the cancer is selected from the group consisting of melanoma, non-small cell lung cancer, head and neck squamous cell cancer, classical Hodgkin lymphoma, primary mediastinal large B-cell lymphoma, urothelial carcinoma, microsatellite instability-high or mismatch repair deficient cancer, microsatellite instability-high or mismatch repair deficient colorectal cancer, gastric cancer, esophageal cancer, cervical cancer, hepatocellular carcinoma, Merkel cell carcinoma, renal cell carcinoma, endometrial carcinoma, a cancer characterized by a tumor having a high mutational burden, cutaneous squamous cell carcinoma, and triple negative breast cancer.

In particular embodiments, the cancer is melanoma. In particular embodiments, the cancer is metastatic melanoma. In particular embodiments, the cancer is primary melanoma.

### VI. Pharmaceutical Compositions, Drug Products and Treatment Regimens

An individual to be treated by any of the methods and products described herein is a human patient diagnosed with a tumor, and a sample of the patient's tumor is available or obtainable to use in testing for the presence or absence of a gene signature biomarker derived using gene expression platform described herein.

The tumor tissue sample can be collected from a patient before and/or after exposure of the patient to one or more therapeutic treatment regimens, such as for example, a PD-1 antagonist, a chemotherapeutic agent, radiation therapy. Accordingly, tumor samples may be collected from a patient over a period of time. The tumor sample can be obtained by a variety of procedures including, but not limited to, surgical excision, aspiration or biopsy.

A physician may use a gene signature score as a guide in deciding how to treat a patient who has been diagnosed with a type of cancer that is susceptible to treatment with a PD-1 antagonist or other chemotherapeutic agent(s). In some embodiments, prior to initiation of treatment with the PD-1 antagonist or the other chemotherapeutic agent(s), the physician will order a diagnostic test to determine if a tumor tissue sample removed from the patient is positive or negative for a gene signature biomarker. However, it is envisioned that the physician could order a first or subsequent diagnostic test at any time after the individual is administered the first dose of the PD-1 antagonist or other chemotherapeutic agent(s). In some embodiments, a physician may be considering whether to treat the patient with a pharmaceutical product that is indicated for patients whose tumor tests positive for the gene signature biomarker. For example, if the reported score is at or above a pre-specified threshold score that is associated with response or better response to treatment with a PD-1 antagonist, the patient is treated with a therapeutic regimen that includes at least the PD-1 antagonist (optionally in combination with one or more chemotherapeutic agents), and if the reported gene signature score is below a pre-specified threshold score that is associated with no response or poor response to treatment with a PD-1 antagonist, the patient is treated with a therapeutic regimen that does not include any PD-1 antagonist.

In deciding how to use the gene signature test results in treating any individual patient, the physician may also take into account other relevant circumstances, such as the stage of the cancer, weight, gender, and general condition of the patient, including inputting a combination of these factors and the gene signature biomarker test results into a model that helps guide the physician in choosing a therapy and/or treatment regimen with that therapy.

The physician may choose to treat the patient who tests biomarker positive with a combination therapy regimen that includes a PD-1 antagonist and one or more additional therapeutic agents. The additional therapeutic agent may be, e.g., a chemotherapeutic, a biotherapeutic agent (including but not limited to antibodies to VEGF, EGFR, Her2/neu, VEGF receptors, other growth factor receptors, CD20, CD40, CD-40L, GITR, CTLA-4, OX-40, 4-1BB, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g., calicheamicin, especially calicheamicin gamma1I and calicheamicin phiI1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate: defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol, pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

The physician may choose to treat the patient who tests biomarker positive with a combination therapy regimen that includes a PD-1 antagonist and hyaluronan degrading enzymes. Administration of PD-1 antagonist can be by any suitable route, and can be facilitated by agents such as hyaluronan degrading enzymes, including hyaluronidases, including soluble PH20 polypeptides, and variants thereof. For systemic administration, the facilitating agents can be modified to increase pharmacological properties, such as serum half-life, by modifying the agents, such as with polymers. See, e.g., U.S. Pat. Nos. 7,767,429, 8,431,380, 7,871,607, International Publication No. WO 2020/022791, U.S. Patent Publication No. US2006/0104968 and European Patent 1858926, and in numerous other patents and publications Exemplary of such agents is the known agent PEGPH20 or rHuPH20. Accordingly, specific embodiments relate to pharmaceutical compositions comprising PD-1 antagonist and any one of a hyaluronan degrading enzyme, hyaluronidase, soluble hyaluronidase, soluble PH20 polypeptide, or a variant of any of the foregoing. In particular embodiments, the pharmaceutical composition comprises PD-1 antagonist and a soluble PH20 polypeptide or a variant thereof. Each therapeutic agent in a combination therapy used to treat a biomarker positive patient may be administered either alone or in a medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy used to treat a biomarker positive patient may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

Each therapeutic agent in a combination therapy used to treat a biomarker positive patient can be administered orally or parenterally, including the intravenous, intramuscular, intraperitoneal, subcutaneous, rectal, topical, and transdermal routes of administration.

A patient may be administered a PD-1 antagonist prior to or following surgery to remove a tumor and may be used prior to, during or after radiation therapy.

In some embodiments, a PD-1 antagonist is administered to a patient who has not been previously treated with a biotherapeutic or chemotherapeutic agent, i.e., is treatment-naïve. In other embodiments, the PD-1 antagonist is administered to a patient who failed to achieve a sustained response after prior therapy with a biotherapeutic or chemotherapeutic agent, i.e., is treatment-experienced.

A therapy comprising a PD-1 antagonist is typically used to treat a tumor that is large enough to be found by palpation or by imaging techniques well known in the art, such as MRI, ultrasound, or CAT scan. In some embodiments, the therapy is used to treat an advanced stage tumor having dimensions of at least about 200 mm^{3,} 300 mm³, 400 mm³, 500 mm³, 750 mm³, or up to 1000 mm³.

Selecting a dosage regimen (also referred to herein as an administration regimen) for a therapy comprising a PD-1 antagonist depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of the PD-1 antagonist that is delivered to the patient consistent with an acceptable level of side effects. Accordingly, the dose amount and dosing frequency depends in part on the particular PD-1 antagonist, any other therapeutic agents to be used, and the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY, Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Biotherapeutic agents used in combination with a PD-1 antagonist may be administered by continuous infusion, or by doses at intervals of, e.g., daily, every other day, three times per week, or one time each week, two weeks, three weeks, monthly, bimonthly, etc. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999). J. Neurol. Neurosurg. Psych. 67:451-456; Portielji et al. (20003) Cancer Immunol. Immunother. 52:133-144.

In certain embodiments, a patient is administered an intravenous (IV) infusion of a medicament comprising any of the PD-1 antagonists described herein, and such administration is part of a treatment regimen employing the PD-1 antagonist as a monotherapy regimen or as part of a combination therapy.

In another embodiment of the invention, the PD-1 antagonist is pembrolizumab, which is administered in a liquid medicament at a dose selected from the group consisting of 200 mg Q3W, 400 mg Q6W, 1 mg/kg Q2W, 2 mg/kg Q2W, 3 mg/kg Q2W, 5 mg/kg Q2W, 10 mg/kg Q2W, 1 mg/kg Q3W, 2 mg/kg Q3W, 3 mg/kg Q3W, 5 mg/kg Q3W, and 10 mg/kg Q3W or equivalents of any of these doses. In some embodiments, pembrolizumab is administered as a liquid medicament which comprises 25 mg/ml pembrolizumab, 7% (w/v) sucrose, 0.02% (w/v) polysorbate 80 in 10 mM histidine buffer pH 5.5, and the selected dose of the medicament is administered by IV infusion over a time period of 30 minutes. The optimal dose for pembrolizumab in combination with any other therapeutic agent may be identified by dose escalation.

The present invention also provides a medicament which comprises a PD-1 antagonist as described above and a pharmaceutically acceptable excipient. When the PD-1 antagonist is a biotherapeutic agent, e.g., a mAb, the antagonist may be produced in CHO cells using conventional cell culture and recovery/purification technologies.

In some embodiments, a medicament comprising an anti-PD-1 antibody as the PD-1 antagonist may be provided as a liquid formulation or prepared by reconstituting a lyophilized powder with sterile water for injection prior to use. WO 2012/135408 describes the preparation of liquid and lyophilized medicaments comprising pembrolizumab, which are suitable for use in the present invention. In some embodiments, a medicament comprising pembrolizumab is provided in a glass vial which contains about 100 mg of pembrolizumab.

These and other aspects of the invention, including the exemplary specific embodiments listed below, will be apparent from the teachings contained herein.

All publications mentioned herein are incorporated by reference for the purpose of describing and disclosing methodologies and materials that might be used in connection with the present invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### EXAMPLES

### Example 1A—Evaluation of Published Signatures and Clinical Trial Data

Merck-Moffitt data set, M2GEN data set, and The Cancer Genome Atlas (TCGA) data set are three molecular profiling data sets of melanoma tumors that were used for analysis. First, Merck-Moffitt melanoma data set was generated as part of Merck-Moffitt Cancer Center collaboration. The Merck-Moffitt data is a comprehensive data set of tumor molecular profiling as well as carefully curated clinical data base. It has over thirty different cancer types represented and over 18,000 tumor samples. These (mostly pre-treatment fresh frozen, processed with NuGEN 50 mg protocol) tumor samples were profiled on Merck custom Affymetrix chip (HRSTA-2.0) using custom Chip Description File (CDF) (GPL10379 in NCBI GEO public repository) at GEL (Gene Expression Laboratory) at Rosetta Inpharmatics (wholly owned subsidiary of Merck & Co., Inc, Rahway, NJ, USA). Out of 21,095 genes represented by probe sets in CDF used, analysis was restricted to 16,120 protein coding genes, with subsequent exclusion of genes with mean and standard deviation below the 25th percentile, leading to 8,728 protein coding genes. This was done to exclude genes with either low expression levels or low variance which would not be expected to yield robust data suitable for biomarker development as well as to control false discovery rate. Identification of candidate genes was done solely using Merck-Moffitt data, more specifically, 724 melanoma tumors, with majority of samples being from metastatic tumor samples—565 (78%), while the rest, 159 (22%) were primary tumor samples. Among 159 primary melanoma tumor samples in Merck-Moffitt data set, 85 (54%) were residual, 29 (18%) were recurrent, 15 (9%) were initial, and 30 (19%) were NOS (Not Otherwise Specified). Among 565 metastatic melanoma tumors in Merck-Moffitt data set, 269 (48%) were distant metastases, 160 (28%) were regional metastases, 2 (0.4%) were local extension, and 134 (24%) were NOS (Not Otherwise Specified). Additional details on clinical sample collection and annotation for Merck-Moffitt data set are provided in ‘Total Cancer Care Protocol: A Lifetime Partnership With Patients Who Have or May be at Risk of Having Cancer (TCCP)’ clinical trial protocol, identifier NCT03977402 found on clinicaltrials.gov, www.moffitt.org/research-science/total-cancer-care/, and Eschrich S A, et al. Enabling Precision Medicine in Cancer Care Through a Molecular Data Warehouse: The Moffitt Experience. JCO Clin Cancer Inform. 2021; 5:561-569. doi:10.1200/CCI.20.00175. Details on molecular profiling, processing, and normalization of data used for analysis are provided in the art. In addition, Merck-Moffitt probe set intensities, generated by using Ref-RMA algorithm as implemented in Affymetrix APT tools/www.affymetrix.com/support/developer/powertools/changelog/index) was summarized on the individual gene level by adding up log 10-transformed intensities over all probe sets annotated with common gene symbol, and further subject to within each individual sample normalization by the 75th percentile evaluated over all protein coding genes within given sample.

Details of publicly available TCGA data set are provided in ‘Genomic Classification of Cutaneous Melanoma’ (The Cancer Genome Atlas Network, Genomic Classification of Cutaneous Melanoma, Cell 161, 1681-1696, Jun. 18, 2015). Tumor gene expression data used for analysis used was taken from TCGA B38 version of Omicsoft TCGA Land (www.arrayserver.com/wiki/index.php?title=Introduction_to_TCGA_Land_Content). Individual gene level Ensembl probe data was used for analysis. For both TCGA and M2GEN, tumor RNA-Seq gene expression data, gene-level FPKM values were converted to log 10 (0.01+FPKM) and subsequently normalized by the 75th percentile calculated over all protein coding genes within each individual sample.

Additional details of proprietary M2GEN Orien Avatar data set, licensed by Merck are available on M2GEN's website (m2gen.com/oncology.com).

TABLE 6
Number of profiled melanoma tumor samples in each data
set, stratified by primary and metastatic tumors.

| | | | Metas- | % | % |
|---|---|---|---|---|---|
| Melanoma | Total | Primary | tatic | Primary | Metastatic |
| Moffitt | 724 | 159 | 565 | 22% | 78% |
| TCGA | 472 | 103 | 369 | 22% | 78% |
| M2GEN | 177 | 54 | 123 | 31% | 69% |

### Example 1B— Endpoints and Description of Statistical Methods Used. Analysis for Both Genes and Signatures

The analyses performed were focused on the relationship between tumor gene expression patterns (individual genes as well as a limited set of pre-specified gene expression signatures as described in ‘Transcriptomic Determinants of Response to Pembrolizumab Monotherapy across Solid Tumor Types’ (Cristescu, et al., Transcriptomic Determinants of Response to Pembrolizumab Monotherapy across Solid Tumor Types, Clin Cancer Res, 28(2) 1680-1689 (2022)) and the following clinical endpoint: metastatic disease versus primary disease.

Metastatic disease status of individual tumor sample was taken directly from patient clinical data provided alongside of molecular profiling data. Analysis of gene expression data association with primary versus metastatic disease was performed using Wilcoxon rank sum test as implemented in ranksum function of Matlab R2020b. All figures and tables show two-sided p-values, nominal as well as FDR (False Discovery Rate) adjusted to account for multiple testing. This adjustment was performed Benjamini & Hochberg method (Benjamini, Y., & Hochberg, Y. Controlling the False Discovery Rate: A Practical and Powerful Approach to Multiple Testing. Journal of the Royal Statistical Society. Series B (Methodological), 57(1), 289-300 (1995)).

Calculation of ROC AUC (Receiver Operating Characteristic Area Under the Curve) was performed using function perfcurve, as implemented in Matlab R2020b. Directionality of association was defined in such a way that values above 0.5 indicate variable (individual gene as well as signature score) to be positively associated, or in other words, up-regulated in metastatic melanoma tumors compared to primary melanoma tumors in given data set.

### Example 1C—Application of Gene Expression Based Biomarker Signature Score

In addition to performing de novo discovery of robustly expressed and statistically significantly differentially between metastatic and primary melanoma tumors genes, a specific set of hypotheses was tested represented by 11 gene expression signature scores introduced in ‘Transcriptomic Determinants of Response to Pembrolizumab Monotherapy across Solid Tumor Types’ (Cristescu, Razvan et al, “Transcriptomic Determinants of Response to Pembrolizumab Monotherapy across Solid Tumor Types” Clinical cancer research; an official journal of the American Association for Cancer Research vol. 28, 8 (2022): 1680-1689) and ‘IFN-γ-related mRNA profile predicts clinical response to PD-1 blockade’ (Ayers et al., IFN-γ-related mRNA profile predicts clinical response to PD-1 blockade; J Clin Invest, 2017 Aug. 1; 127(8):2930-2940. doi: 10.1172/JCI91190) Details on the gene lists associated with each of 11 gene expression signatures, methods of calculating signature score in individual samples are provided in ‘Transcriptomic Determinants of Response to Pembrolizumab Monotherapy across Solid Tumor Types’ (Cristescu et al, 2022).

As shown in Table 7, several of the 11 gene expression-based variables tested have statistically significant differential expression between metastatic and primary melanoma tumors Given the smaller number of samples in M2GEN data (n=177 samples total), compared to Merck-Moffitt and TCGA, it should be expected that weaker association would be observed in M2GEN data compared to that of TCGA (472 samples total), and especially Merck-Moffitt (724 samples total). Table 7 shows the statistical significance of differential expression between metastatic and primary melanoma tumors in the three data sets (Merck-Moffitt, TCGA, and M2GEN) for 11 pre-specified gene expression signatures. Numerical values shown are ROC AUC as well as signed log 10-transformed two-sided nominal p-value by Wilcoxon rank sum test. Directionality of signed log 10 p-value is chosen to be such that positive values correspond to ROC AUC >0.5 associated with up-regulation in metastatic tumors compared to primary, and vice versa: negative value for ROC AUC <0.05 indicating down-regulation in metastatic tumors. For example, signed log 10 p-value of absolute value above 1.0, 2.0, and 3.0 corresponds to p-value<0.1, <0.01, and <0.001 respectively.

TABLE 7
Statistical significance of differential expression between metastatic and primary
melanoma tumors in three data sets for 11 pre-specified gene expression signatures.

| | Metastatic versus Primary | | | | | |
|---|---|---|---|---|---|---|
| | ROC AUC | | | signed log10 p-value | | |
| | Merck- | | | Merck- | | |
| Melanoma Tumors | Moffitt | TCGA | M2GEN | Moffitt | TCGA | M2GEN |
| GEP | 0.55 | 0.62 | 0.58 | 1.44 | 3.71 | 1.00 |
| mMDSC | 0.57 | 0.68 | 0.60 | 2.25 | 7.53 | 1.37 |
| gMDSC | 0.47 | 0.45 | 0.55 | −0.54 | −1.06 | 0.56 |
| Angiogenesis | 0.42 | 0.64 | 0.51 | −2.59 | 5.12 | 0.07 |
| Glycolysis | 0.37 | 0.36 | 0.50 | −6.58 | −5.11 | −0.03 |
| Hypoxia | 0.43 | 0.41 | 0.53 | −1.98 | −2.09 | 0.32 |
| MYC | 0.60 | 0.41 | 0.61 | 3.75 | −2.09 | 1.70 |
| Proliferation | 0.65 | 0.55 | 0.62 | 7.81 | 0.97 | 1.91 |
| RAS | 0.55 | 0.41 | 0.55 | 1.43 | −2.15 | 0.48 |
| Stroma/EMT/TGFbeta | 0.26 | 0.53 | 0.33 | −20.11 | 0.55 | −3.33 |
| WNT | 0.35 | 0.44 | 0.43 | −8.12 | −1.09 | −0.89 |

### Example 1D—Results

Univariate analysis of differential gene expression between metastatic versus primary melanoma tumors in Merck-Moffitt data set identified many significantly differentially expressed genes, even after applying Benjamini & Hochberg correction. Out of 8,728 protein coding genes tested, 4,697 had two-sided FDR-adjusted p-value<0.01, of which 2,717 were up-regulated in metastatic tumors compared to primary (ROC AUC>0.5), and the remaining 1,980 genes were down-regulated (ROC AUC<0.5). The resulting list of genes found to be statistically differentially expressed between metastatic versus primary melanoma tumors in Merck-Moffitt data set was further refined to two lists: 128 genes that had FDR-adjusted two-sided p-value<0.01 and ROC AUC>0.7, that were up-regulated in metastatic tumors, and complementary list of 513 genes down-regulated in metastatic tumors that had FDR-adjusted two-sided p-value<0.01 and ROC UC<0.3.

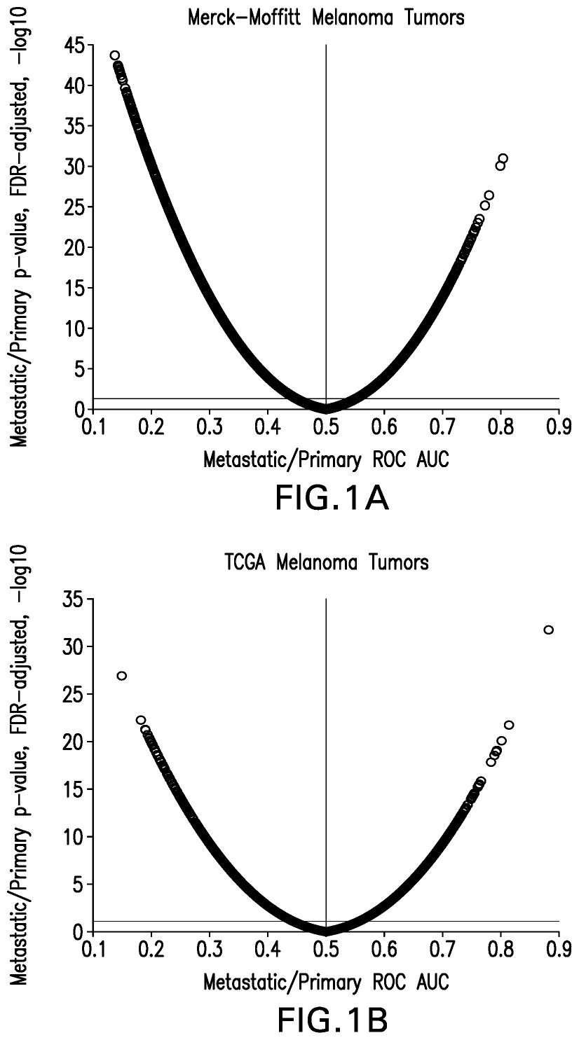

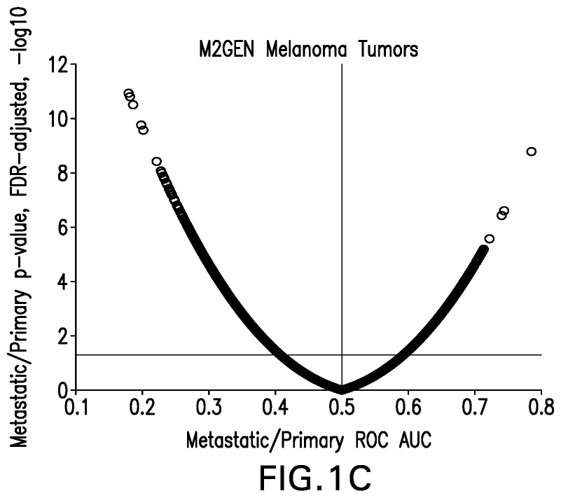

FIGS. 1A, 1B, and 1C show relationship between ROC AUC for metastatic versus primary tumors across all genes screened within the three data sets compared against FDR-adjusted p-value, shown on −log 10 scale.

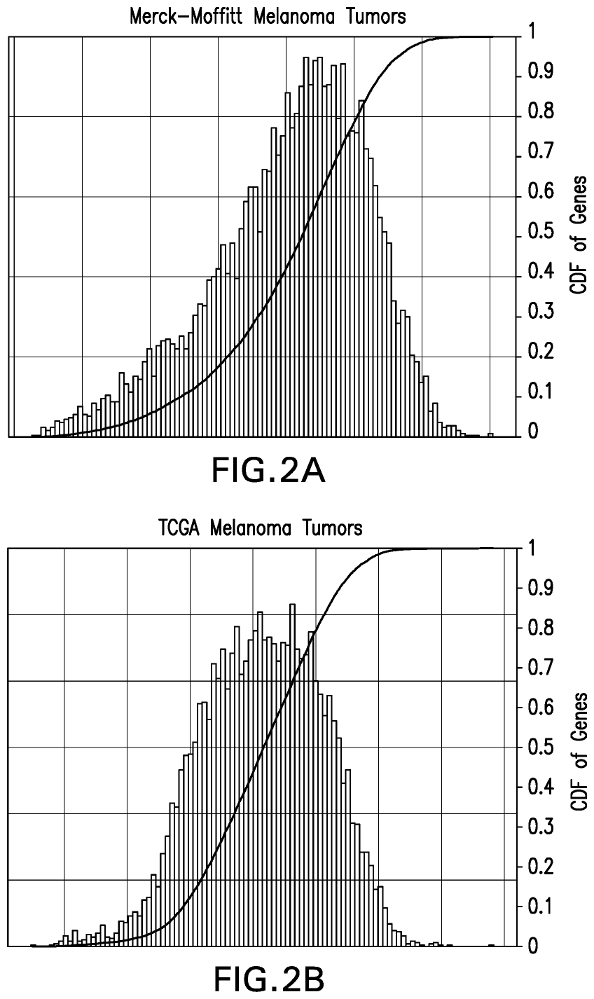

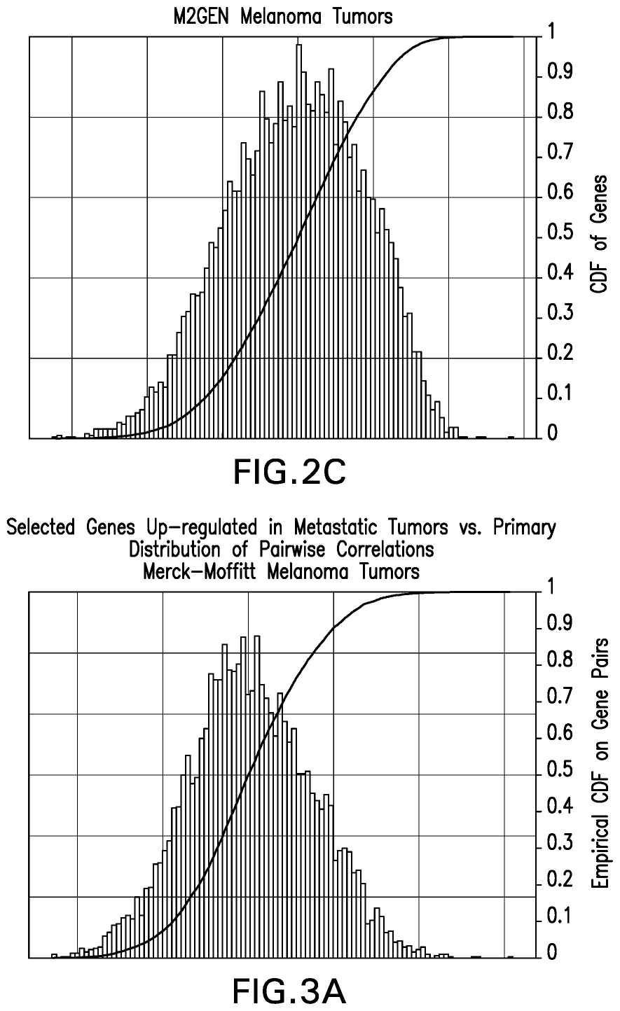

FIGS. 2A, 2B, and 2C show a comparison of the distribution ROC AUC for metastatic versus primary tumors across all genes screened within the three data sets.

Similar to Merck-Moffitt data, strong genome wide differential expression between metastatic and primary melanoma tumors was also observed in TCGA and M2GEN data sets. In TCGA, 3,197 genes (out of 8,728) were observed to have FDR-adjusted two-sided p-value<0.01, of which 1,978 were up-regulated in metastatic tumors (129 out of 1,978 had ROC AUC >0.7), and 1,219 were down-regulated (139 genes out of 1,219 had ROC AUC<0.3). In M2GEN melanoma tumors, given the smaller sample size compared to Merck-Moffitt and TCGA data sets, the number of differentially expressed genes between metastatic and primary melanoma tumors was still highly statistically significant: 506 genes with two-sided FDR adjusted p-value<0.01, 147 genes up-regulated (19, out of 147, with ROC AUC>0.7), and 359 genes down-regulated (with 138, out of 359, having ROC AUC<0.3).

Two selected gene sets identified in Merck-Moffitt data set (128 genes up-regulated and complementary 513 genes down-regulated in metastatic melanoma tumors) show consistent directionality of up- and down-regulation in metastatic versus primary in the other two data sets. Out of 128 genes, 109 (85%) are up-regulated in TCGA (ROC AUC >0.5), and 103 (80%) are up-regulated in M2GEN. Out of the 513 genes, 440 (86%) are down-regulated in TCGA (ROC AUC <0.5), and 503 (98%) are down-regulated in M2GEN. In addition to consistency in the directionality of up or down regulation in metastatic versus primary melanoma tumors, good concordance was observed in terms of p-values for the 128 genes, 81 (63%) were significant in TCGA, and 48% are significant in M2GEN. For the 513 genes, observed concordance was even stronger. 389 (76%) were significant in TCGA, and 437 (85%) were significant in M2GEN. Significance was defined as nominal p-value by Wilcoxon rank sum test below 0.05.

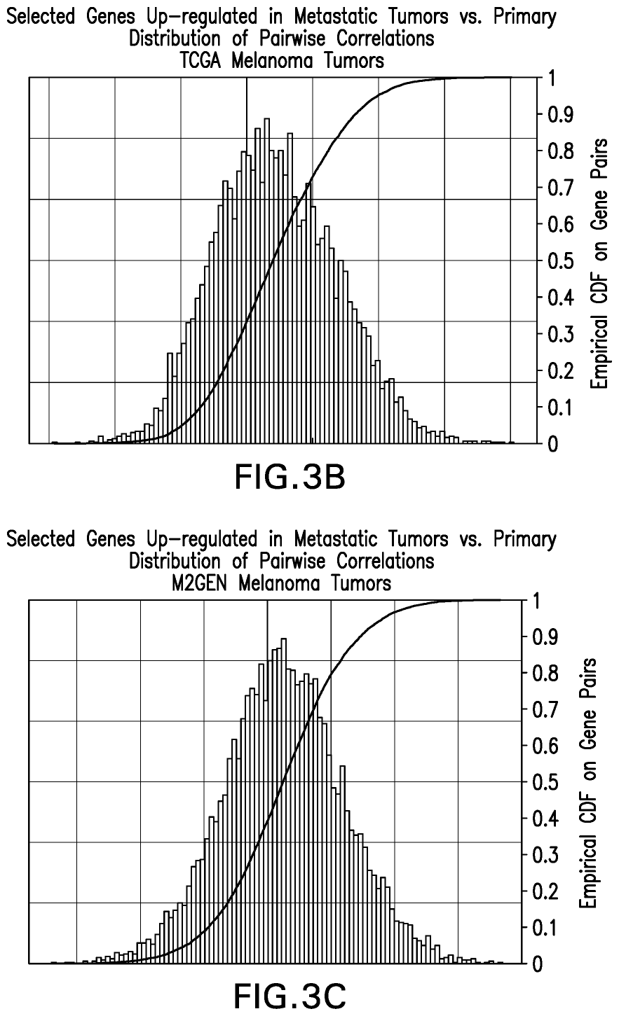

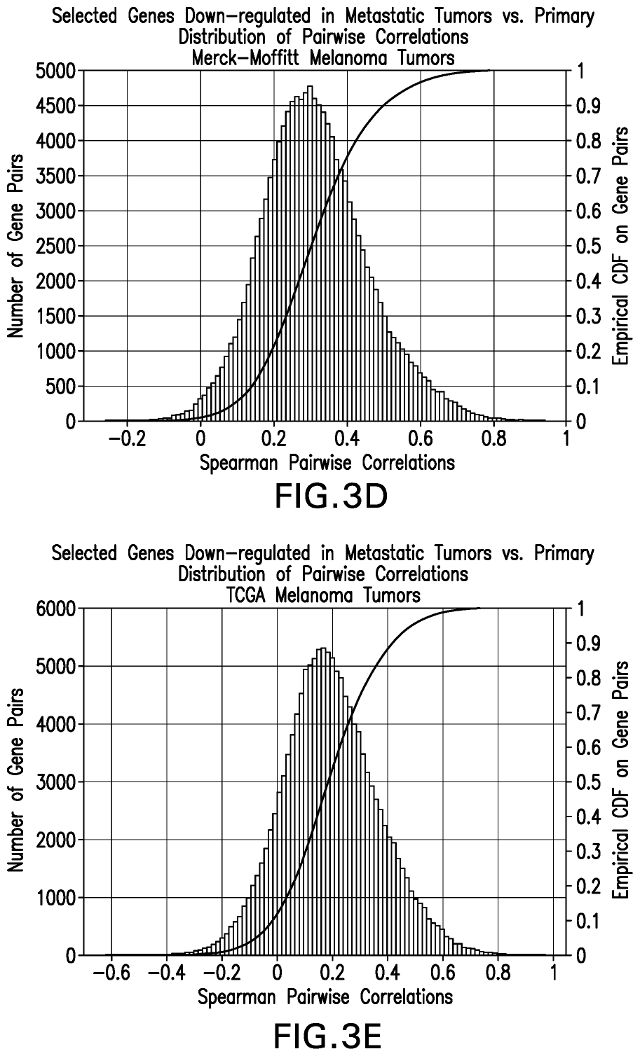

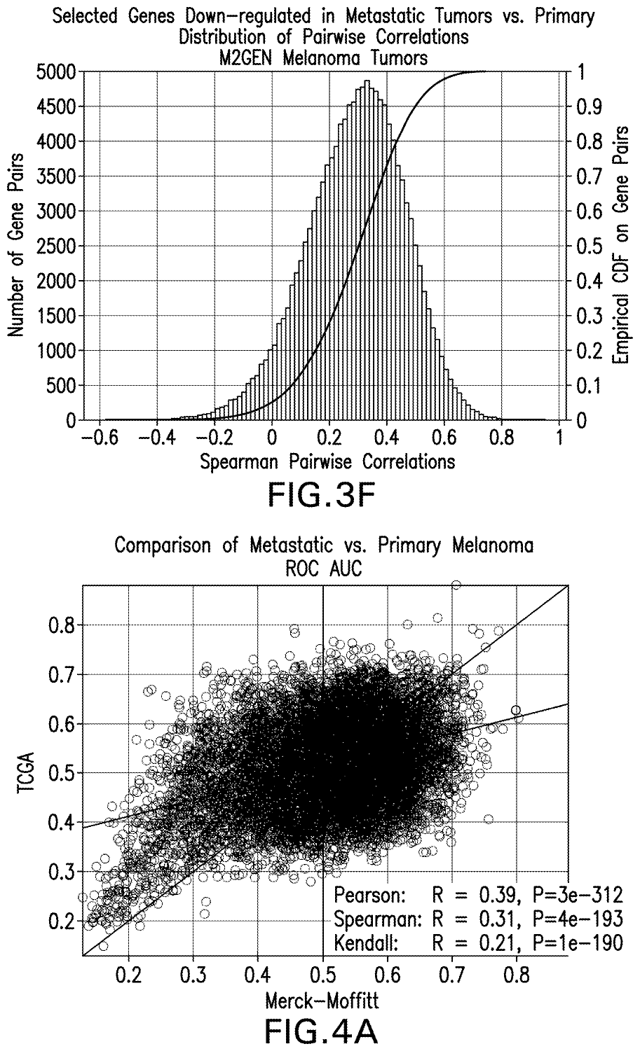

Each set of genes was observed to be coherent and consisting of co-expressed genes, as shown in FIG. 3. Among 123 genes up-regulated in metastatic melanoma tumors, over 90/6 and 70% of all pairwise correlations were positive in Merck-Moffitt and in TCGA data sets respectively. Among 513 genes down-regulated in metastatic tumors, over 95%, 90%, and 95% of all pairwise correlations were positive in Merck-Moffitt, TCGA, and M2GEN data sets respectively.

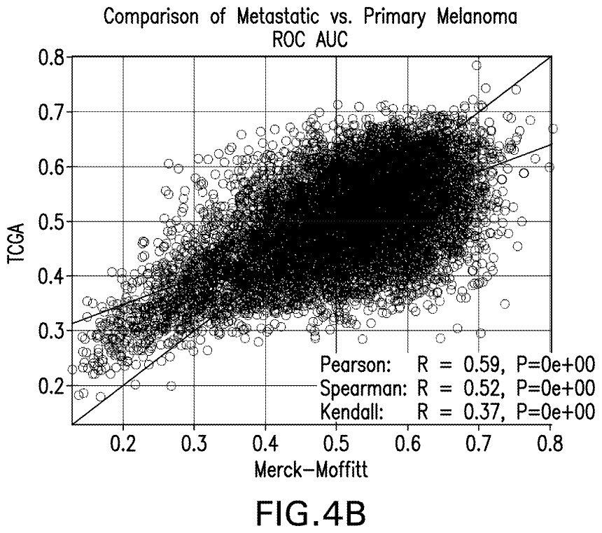

Also, as can be seen in FIG. 4, a high degree of concordance in differential gene expression between metastatic versus primary melanoma tumors, can be observed between what was determined in Merck-Moffitt data and TCGA, as well as M2GEN melanomas, especially among genes down-regulated in metastatic melanomas.

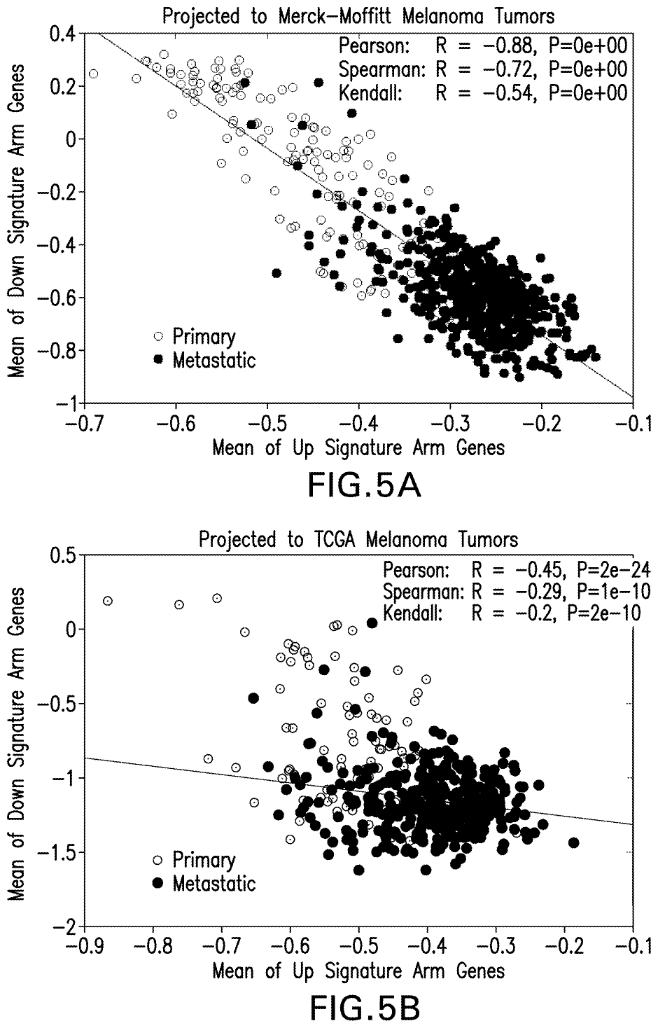

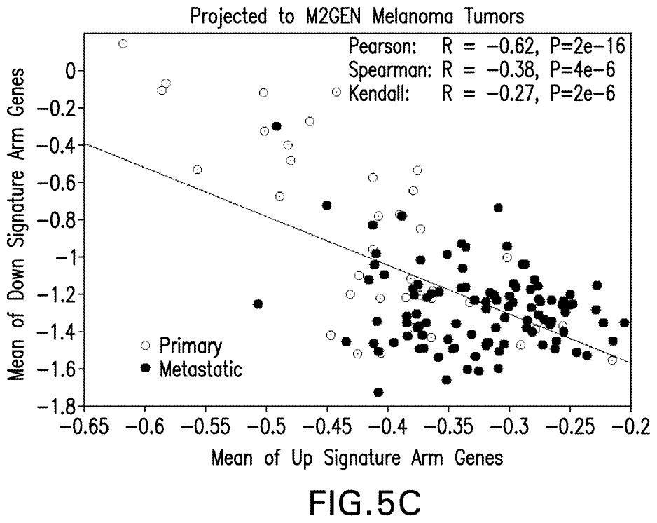

Additionally, as shown on FIG. 5, these two sets of genes were anti-correlated, as can be seen when plotting corresponding signature scores, defined as gene set mean values evaluated in each tumor sample. This in turn supports using the difference in mean expression calculated separately for selected genes found to be up-regulated in metastatic samples and those that were down-regulated as a biomarker, whose values are differentially expressed between primary and metastatic tumors, and are confirmed to be so, when tested and validated in two independent melanoma tumor data sets, not used for biomarker development, such as TCGA and M2GEN melanoma tumors.

FIGS. 5A, 5B, and 5C are are scatterplots that show the coherence of genes selected by differential expression between metastatic versus primary tumors in Merck-Moffitt Melanomas observed in expression data in three data sets. Each plot shows signature-up score, defined as mean expression of selected set of genes found to be statistically significantly up-regulated in Merck-Moffitt metastatic tumors versus primary tumors on x-axes versus signature-down score, defined as mean expression of complementary set of genes selected for being statistically significantly down-regulated in Merck-Moffitt metastatic melanoma tumors compared to primary. Each dot represents a tumor sample in a given data set, labeled by the tumor type (primary or metastatic). Robust linear regression fitted line is shown as well as three correlation coefficients and associated p-values (Pearson, Spearman, and Kendall's tau) FIG. 5A displays the observed relationship between two scores evaluated in Merck-Moffitt data. FIGS. 5B and 5C show results for TCGA and M2GEN respectively.

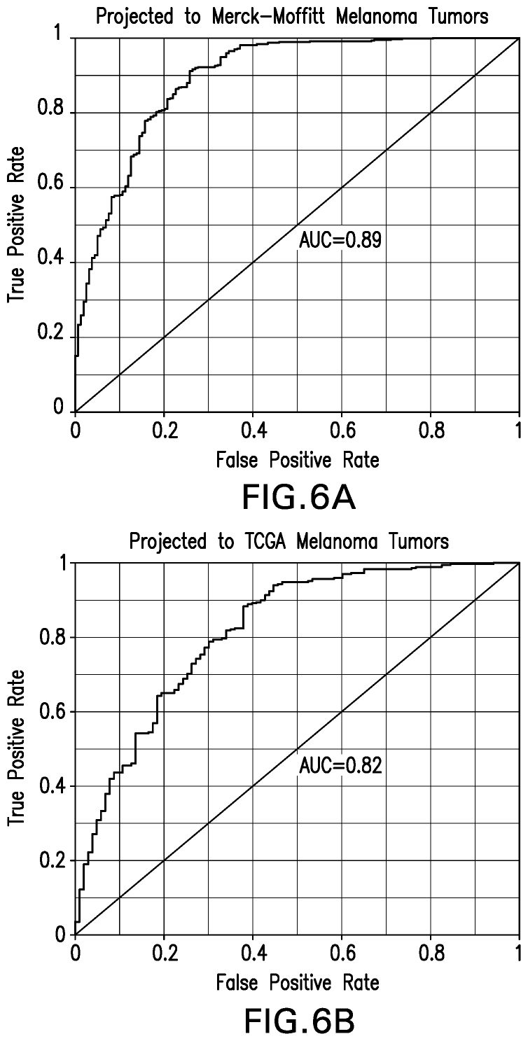

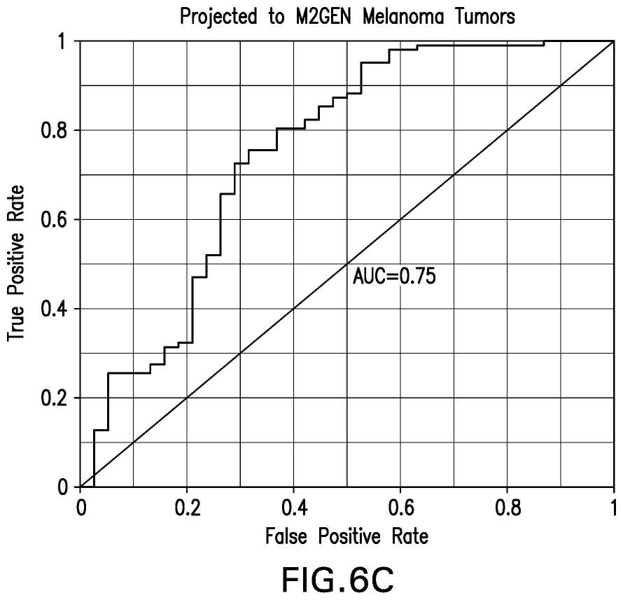

When tested on independent melanoma tumor samples, our proposed gene expression signature was shown to have ROC AUC=0.82 and 0.75 on TCGA and M2GEN data sets respectively (FIG. 6A-C).

FIGS. 6A, 6B, and 6C are ROC AUC curves describing the association between proposed gene expression signature score and metastatic versus primary status in each individual data set. Given the fact that two selected complementary gene sets, used to calculate gene expression signature score, were derived on Merck-Moffitt data only, FIG. 6A represents the case of back-substitution, whereas FIG. 6B and FIG. 6C represent testing on independent data sets not used to develop the signature being tested (Merck-Moffitt, TCGA, and M2GEN in FIG. 6A, FIG. 6B, and FIG. 6C respectively).

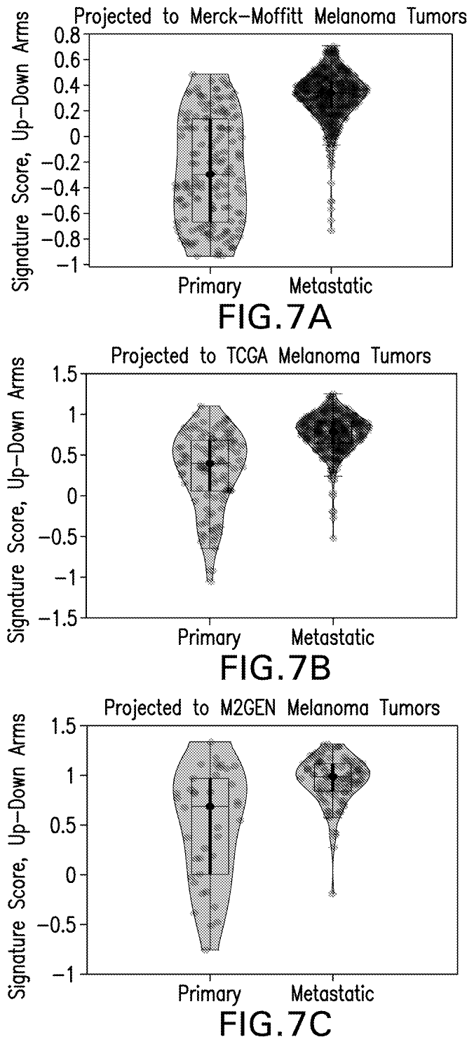

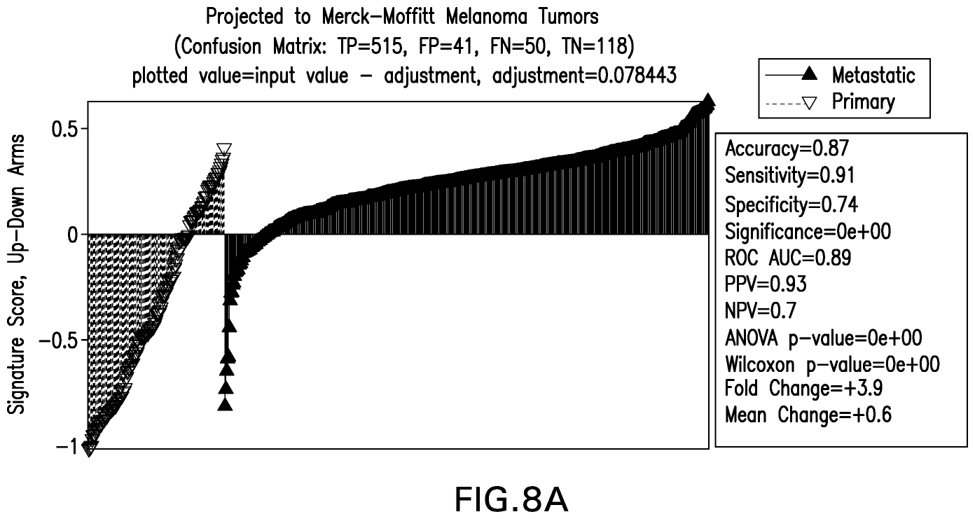

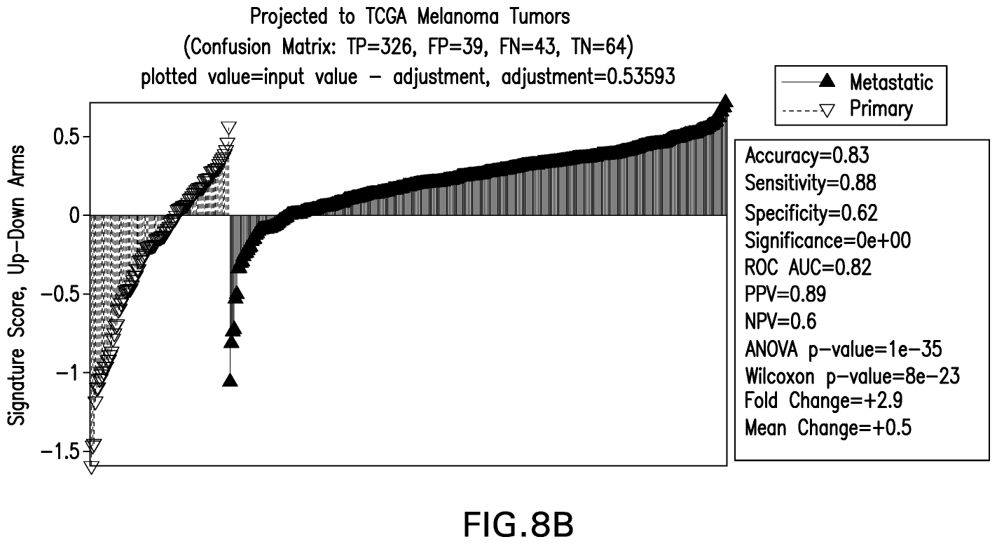

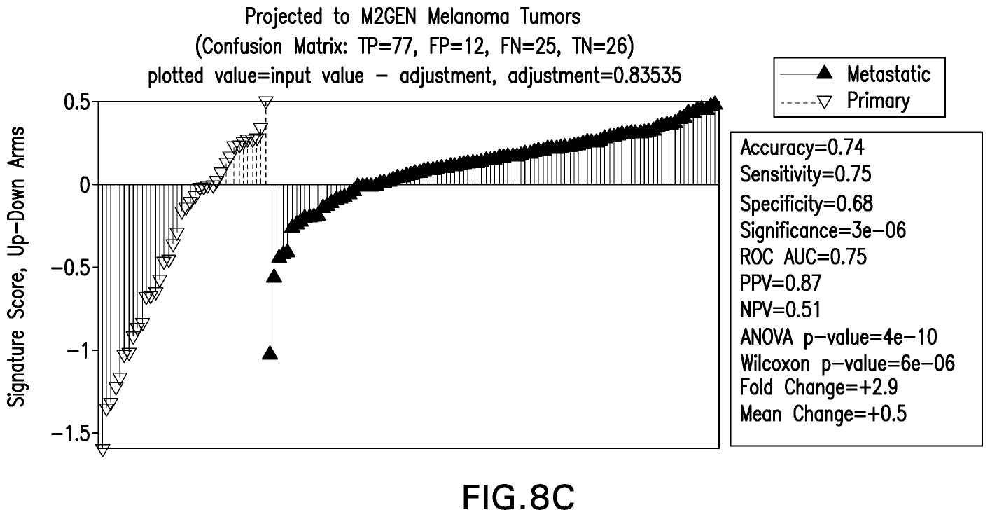

Some primary tumors were observed to have signature score values representative of metastatic tumors (FIG. 7 and FIG. 8). FIGS. 7A. 7B, and 7C are superimposed violin and boxplots illustrating the distributions of proposed gene expression signature score with and between primary and metastatic melanoma tumors in each data set. Distributions in Merck-Moffitt, TCGA, and M2GEN are shown in 7A, 7B, and 7C respectively. Each plotted value (dot) represents a tumor sample and y-axis displays the value of the signature score evaluated in given sample. On each box, the central mark indicates the median, and the bottom and top edges of the box indicate the 25th and 75th percentiles, respectively. The whiskers extend to the most extreme data points not considered outliers, and the outliers are plotted individually using the ‘+’ marker symbol.

FIGS. 8A, 8B, and 8C are sorted waterfall plots illustrating distributions and difference in distributions of proposed gene expression signature scores between metastatic and primary melanoma tumors. Each stem and dot represent individual tumor sample. Primary melanoma tumors are grouped on the left, followed by metastatic melanoma tumors on the right. Y-axes value show gene expression signature scores after applying baseline adjustment calculated as signature score evaluated at the cutoff corresponding to Youden index on ROC curve. TP, FP, FN, and TN abbreviations correspond to the number of True Positives, False Positive, False Negative, and True Negative samples observed at given signature score cutoff. PPV and NPV stands for Positive Predictive Value and Negative Predictive Value respectively. Significance represents Fisher exact test p-value obtained at the specified cutoff. Mean change is equal to the difference in score means between two sets (metastatic versus primary) for signature score (evaluated on log 10-scale), and Fold Change is the ratio in means for two sets on nominal scale.

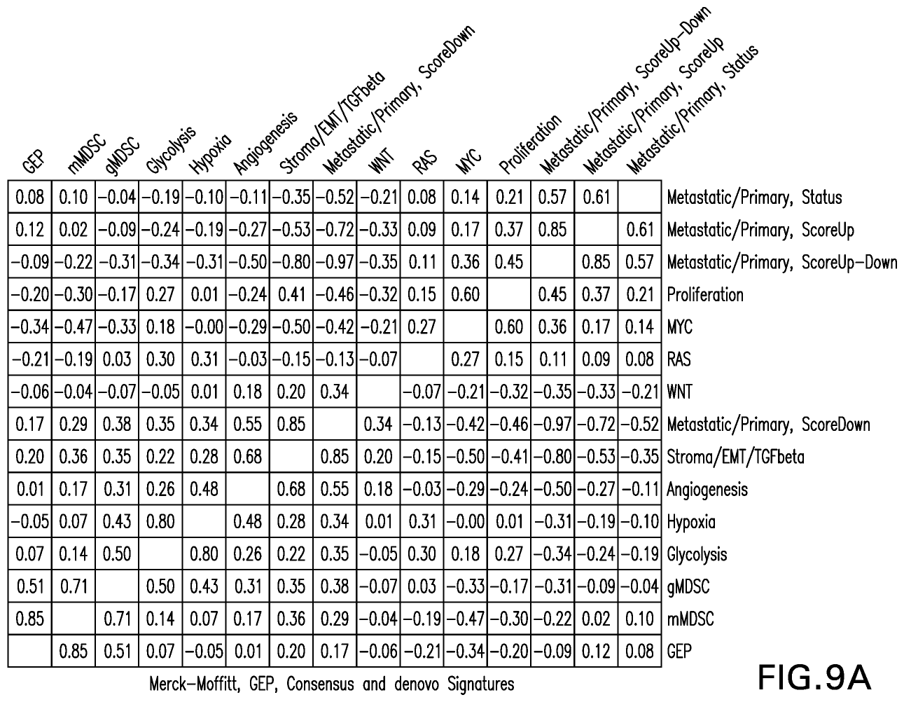

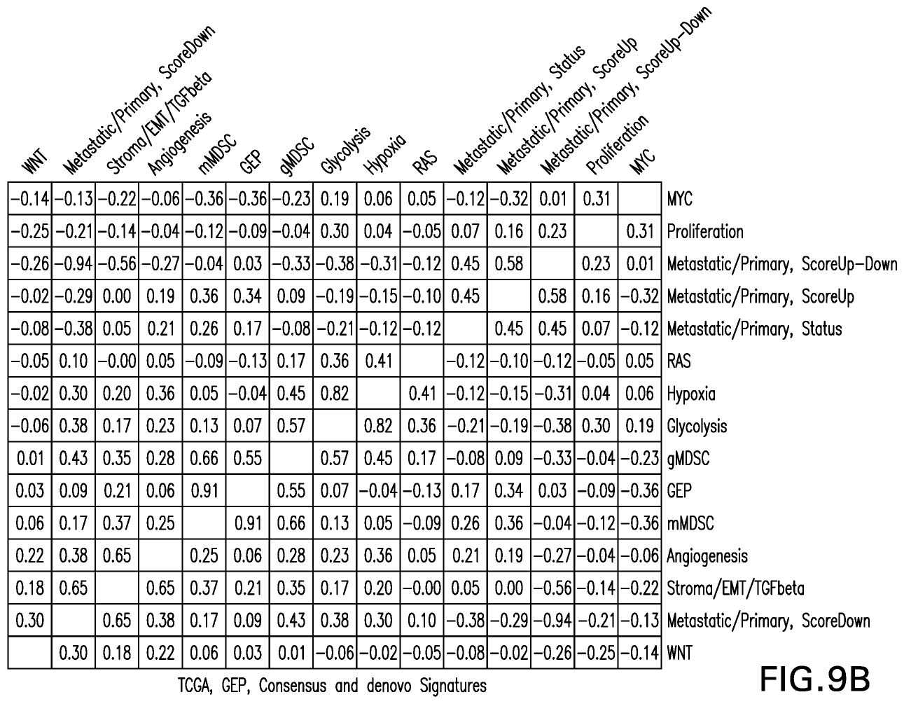

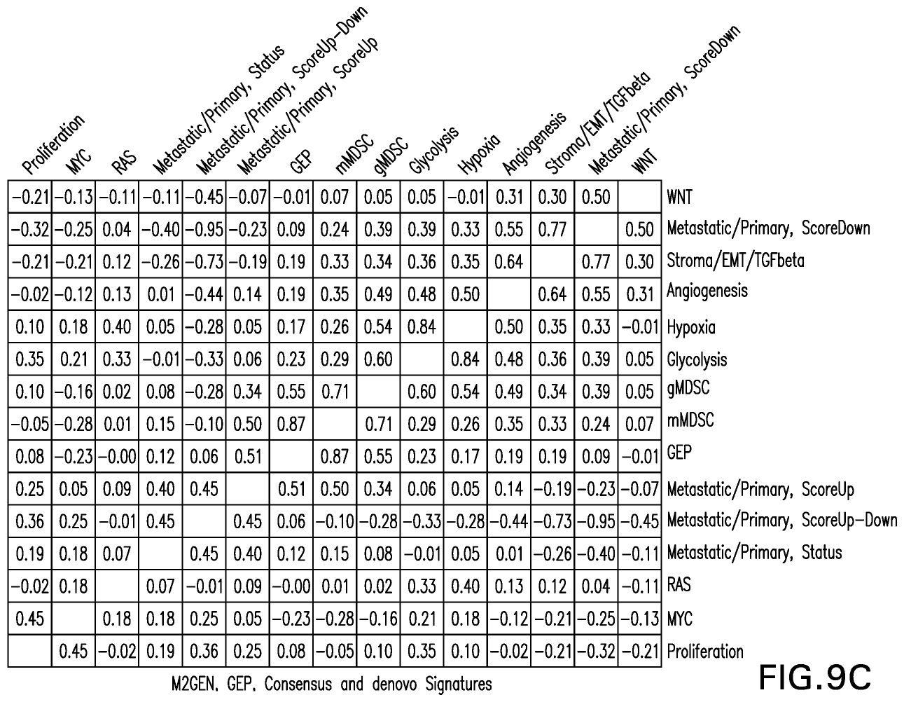

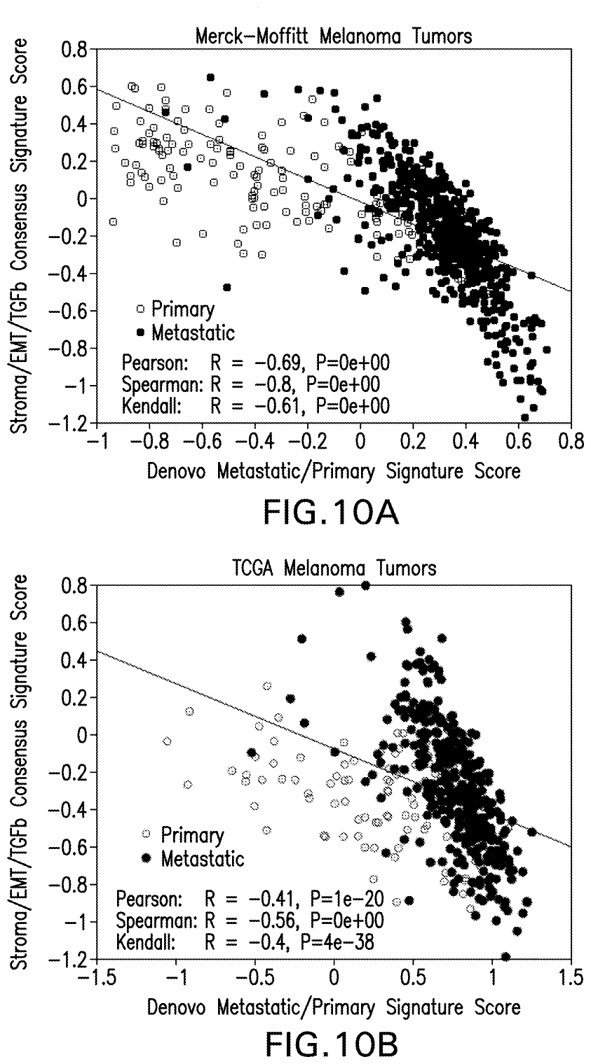

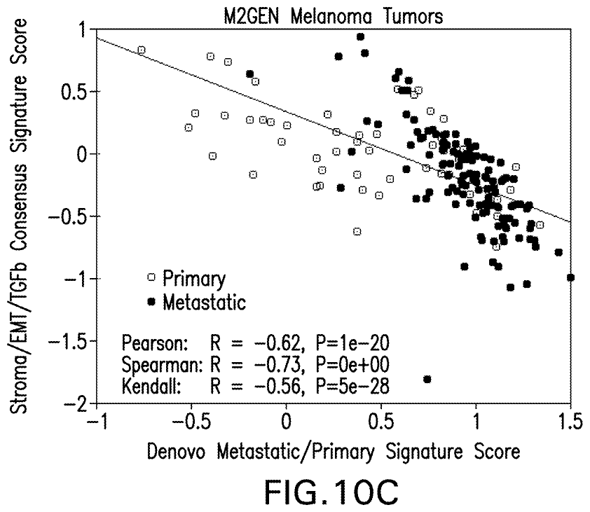

When compared to 11 previously selected (and tested) GEP and consensus signatures, observed to be differentially expressed between metastatic and primary melanoma tumors, FIG. 9 shows that neither up, down, or up-down proposed de novo gene signature scores are highly correlated to prior patterns tested, and thus can be proposed as independent predictors of metastatic potential in primary melanoma tumors.

FIGS. 9A, 9B, and 9C are two-dimensional heat map plots showing correlations among metastatic versus primary status, proposed denovo signature scores, and additional gene expression signatures. They show Spearman correlation coefficients among 11 pre-selected signature scores (T-cell inflamed GEP (Ayers et. al., (2017) IFN-γ-related mRNA profile predicts clinical response to PD-1 blockade; J Clin Invest, 2017 Aug. 1; 127(8):2930-2940) and 10 consensus signatures, (Gastman, R. et al., (2019) Identification of patients at risk of metastasis using a prognostic 31-gene expression profile in subpopulations of melanoma patients with favorable outcomes by standard criteria. J Am Acad Dermatol, vol. 80, 1, 149-157), together with three de novo metastatic versus primary melanoma signature scores (up arm score, down arm score, and the up-down signature score, evaluated as the difference between up and down signature score), along with binary variable indicating metastatic (=1) versus primary (═O) status for each tumor sample in the corresponding data set. Ordering of rows and columns is the same and was determined by hierarchical clustering based on Euclidean distance metric and Ward's linkage. Greyscale color range spans correlation values from −1 (black) to +1 (white, observed on the main diagonal depicting self-correlation). Values of Spearman correlation coefficient between two variables at the intersection of labeling corresponding row and column, rounded to two decimal points, are overlaid. FIGS. 9A, 9B, and 9C correspond to observer pairwise correlations observed in Merck-Moffitt, TCGA, and M2GEN melanoma tumors, respectively, clustered within each data set.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

1. A method of determining the prognosis of a patient who has been diagnosed with melanoma, which comprises:
(a) obtaining or receiving a sample from a tumor from the patient,
(b) determining the patient's gene expression based biomarker profile by determining the expression of 5 or more genes listed in Table 1 (up-regulated gene signature) or 5 or more genes listed in Table 2 (down-regulated gene signature) in the sample,
(c) determining a signature score from the gene expression based biomarker, wherein
(i) for the up-regulated gene signature, if the calculated signature score is equal to or greater than a pre-specified threshold, then the tumor is classified as biomarker positive, and if the calculated signature score is less than the pre-specified threshold, then the tumor is classified as biomarker negative, and
(ii) for the down-regulated gene signature, if the calculated signature score is equal to or less than a pre-specified threshold, then the tumor is classified as biomarker positive, and if the calculated signature is greater than the pre-specified threshold, then the tumor is classified as biomarker negative, and
wherein the patient is determined to have a poor prognosis if the tumor is classified as biomarker positive or a favorable prognosis if the tumor is biomarker negative.

2. (canceled)

3. A method for testing a tumor from a patient for the presence or absence of a biomarker that predicts clinical need for further treatment with a PD-1 antagonist, which comprises:
(a) obtaining or receiving a sample from the patient's tumor,
(b) measuring the raw RNA expression level in the tumor for each gene in a gene expression based biomarker;
(c) normalizing each of the measured raw RNA expression levels;
(d) calculating the arithmetic mean of the normalized RNA expression levels for each of the genes to generate a score for the gene expression based biomarker;
(e) comparing the calculated score to a reference score of the gene expression based biomarker; and
(f) classifying the tumor as biomarker positive or biomarker negative;
wherein the gene expression based biomarker comprises
(i) at least 5 genes selected from the genes listed in Table 1 which have a positive correlation to the signature score,
(ii) at least 5 genes selected from the genes listed in Table 2, which have a negative correlation to the signature score, or
(iii) a combination of at least 5 genes selected from the genes listed in Table 1 having a positive correlation to the signature score and/or the genes listed in Table 2 having a negative correlation to the signature score;
wherein a tumor is biomarker positive if the calculated score is higher than the reference score of the gene expression based biomarker, and wherein a tumor is biomarker negative if the calculated score is lower than the reference score of the gene expression based biomarker, and
wherein a biomarker positive tumor indicates a need for further treatment with a PD-1 antagonist and a biomarker negative tumor does not indicate a need for further treatment with a PD-1 antagonist.

4. The method of claim 3, wherein step (b) further comprises normalizing each of the measured raw RNA levels for each gene in the gene expression based biomarker using the measured RNA levels of a set of normalization genes.

5. The method of claim 4, wherein the set of normalization genes comprises 10-12 housekeeping genes.

6. The method of claim 4, wherein the set of normalization genes comprises at least ten of the genes from Table 3.

7. A method for treating melanoma in a patient having a tumor which comprises administering to the patient a PD-1 antagonist if the tumor is positive for a gene expression based biomarker; wherein the determination of whether the tumor is positive or negative for the gene expression based biomarker was made using a method according to claim 3.

8. A method for treating melanoma in a patient having a tumor which comprises administering to the patient a PD-1 antagonist if the patient is determined to have a poor prognosis, wherein the determination of whether the patient has a favorable or poor prognosis was made using a method according to claim 1.

9. A method for treating melanoma in a patient having a tumor which comprises:
(a) determining if the tumor is positive or negative for a gene expression based biomarker, wherein the determining step comprises:
(i) obtaining a sample from the patient's tumor;
(ii) sending the tumor sample to a laboratory with a request to test the sample for the presence or absence of the gene expression based biomarker;
(iii) receiving a report from the laboratory that states whether the tumor sample is biomarker positive or biomarker negative, wherein the determination of whether the tumor sample is biomarker positive or biomarker negative is determined by a method according to claim 3 and
(b) administering to the patient a PD-1 antagonist if the tumor is positive for the biomarker.

10. The method of claim 9, wherein the positive biomarker status is determined by calculating the expression of 5 or more up-regulated genes selected from the group comprising: ABHD10, ABHD3, ACVR2B, ADAL, ALG13, ANGEL1, ATG16L1, B4GALT3, BRAF, BRSK1, C12orf60, C1orf56, C4A, C7, CCDC151, CCDC93, CCNE1, CD1D, CD38, CD5L, CDC42SE1, CHEK2, CHORDC1, CMTM7, CPOX, CR1, CRELD1, CRNKL1, CSE1L, DARS2, DBNDD2, DDIT4, DEFB108B, DHODH, DNAJB9, DNAJC5B, DPM3, DTNB, EIF4A2, ERP29, ESM1, EXOC4, FAM122B, FANCL, FMNL2, FUBP1, GGA2, GHRH, GLUL, GPN3, HBE1, HELB, HEMK1, INPP5B, KCNJ10, L3MBTL1, LHFPL1, LIPT1, MAGED1, MBOAT1, MDM1, MERTK, METTL3, METTL7B, MGAT4A, MMD, MPI, MRM1, MSH6, MSI2, MSL2, NAPB, NBPF1, NDUFAF3, NLK, NT5DC3, OLIG2, OMA1, OXNAD1, P4HA1, PDIA4, PGBD2, PHF6, PIP5K1A, PMS2, POLR3K, PREPL, RAB3GAP2, RBM39, RBM45, RNF2, RRN3, SEC24A, SFXN2, SIGLEC11, SLC30A3, SNAPC3, SPAG4, SPIN3, SRPRB, SRSF9, STRBP, STX16, SYS1, TAF1A, TGM2, THOC2, TMEM182, TMEM81, TOP1, TP53BP1, TRIM5, TRNT1, TRPM2, UBFD1, URB2, VRK3, WDR76, WDSUB1, XPO1, ZMYND8, ZNF189, ZNF26, ZNF337, ZNF544, ZNF550, ZNF572, and ZNF841.

11. The method of claim 9, wherein the positive biomarker status is calculated by determining the expression level of 5 or more down-regulated genes selected from the group comprising: A4GALT, ABLIM1, ADAM15, ADAM33, ADAMTS12, ADAMTS2, ADAMTS5, ADK, AGTR1, AHNAK, AHNAK2, AKR1C1, AKR1C2, AKR1C3, ALDH3A1, ALDH3B2, ALOXE3, ALS2CL, ANGPTL2, ANO1, ANPEP, ANXA2, ANXA9, APCDD1, APLNR, AQP1, AQP3, AQP5, ARHGEF15, ARHGEF19, ARHGEF4, ARL4D, ARNTL2, ASAP3, ASPN, ASPRV1, ATL3, ATP12A, ATP6V1C2, ATP8B1, B3GNT4, BDKRB2, BICC1, BICD2, BMP1, BMPR2, BOC, BSPRY, BTBD11, C12orf54, C19orf33, CA12, CALML3, CALML5, CAPN1, CAPNS2, CASZ1, CBLC, CCDC113, CCDC120, CCDC3, CCDC92, CCL22, CD109, CD24, CD248, CD34, CD44, CD9, CDA, CDH13, CDH3, CDHR1, CDR1, CDS1, CEACAM19, CH25H, CLDN1, CLDN4, CLEC14A, CLIC3, CLTB, CNFN, COL12A1, COL13A1, COL14A1, COL15A1, COL17A1, COL18A1, COL1A1, COL1A2, COL23A1, COL3A1, COL5A1, COL5A2, COL5A3, COL6A1, COL6A2, COL6A3, COL6A6, COL7A1, COL8A2, COMP, COMTD1, CPA3, CPA4, CPXM1, CPXM2, CPZ, CRABP1, CRABP2, CRCT1, CREB3L1, CRISPLD2, CRYM, CST6, CSTA, CTNNBIP1, CTSG, CTSK, CTTNBP2NL, CXADR, CXCL12, CXCL14, CYB561, CYB5R3, CYP26B1, CYP2S1, CYYR1, DAPL1, DAZAP2, DCN, DEGS1, DEGS2, DENND2C, DGAT2, DHRS1, DIO2, DMKN, DPP4, DPT, DSC2, DSEL, DSP, DST, DUOX1, DUOXA1, DUSP14, EBF1, ECSCR, EDN1, EFNA3, EFNB2, EGLN3, EHD2, ELMO3, ELOVL3, ELOVL4, ELOVL7, EML1, EMP1, EMP2, EN1, EPHA1, EPHB6, EPHX3, EPPK1, EPS8L1, ERBB2, ESRP2, ETS2, EVPL, EXPH5, F10, F2RL1, F2RL2, FADS6, FAM110C, FAM167A, FAM180A, FAM83F, FAM83H, FAT2, FAT4, FBLN1, FBLN2, FBN1, FCER1A, FGF11, FGFR3, FIBIN, FMO1, FOSL2, FOXQ1, FUT1, FZD10, GALNT1, GAN, GAS1, GDPD3, GJA1, GJB2, GJB3, GJB5, GJB6, GLT8D2, GLTP, GNA15, GNAL, GPC1, GPR68, GREM1, GRHL1, GRHL2, GSDMA, HAS3, HDC, HEBP2, HES2, HOPX, HOXD10, HR, HSD11B2, HSPA12B, HTRA1, ID1, IDE, IFF02, IGFBP4, IGFL2, IGFL4, IL1R1, IL1RN, IL20RB, IMPA2, IRX2, IRX3, IRX5, ISM1, ITGB4, IVL, JAM2, JMJD7-, LA2G4B, JUP, KCND3, KCNK6, KCNK7, KCTD11, KIAA1217, KIAA1522, KIF26A, KIT, KITLG, KLC3, KLF10, KLF11, KLF3, KLF4, KLF5, KLF6, KLK10, KLK5, KLK6, KLK8, KRT1, KRT10, KRT15, KRT17, KRT19, KRT2, KRT23, KRT31, KRT5, KRT78, KRT79, KRT80, KRTAP10-12, KRTDAP, LAD1, LAMA2, LAMA3, LAMB3, LCE1A, LCE1B, LCE1D, LCE1F, LCE2A, LCE3A, LCN2, LIMA1, LOXL1, LRRC15, LRRC32, LRRC8E, LTB4R, LTBP1, LUM, LY6D, LY6G6C, LYNX1, LYPD2, LYPD3, LYPD5, MAL2, MALL, MAP7, MARVELD1, MAST4, MEGF6, MEOX1, MFAP4, MFAP5, MICALL1, MINK1, MMP11, MMP2, MMP7, MMRN2, MN1, MPZL2, MRGPRF, MSX2, MXRA5, MXRA8, MYO6, NCCRP1, NDRG4, NDUFA4L2, NEURL1B, NFATC4, NGEF, NIPAL4, NKD2, NLRX1, NMU, NRARP, NTF3, NTN1, NUAK1, OLFM2, OLFML1, OLFML2A, OSR2, OTUB2, OVOL1, PAK6, PALLD, PALMD, PAPPA, PAQR7, PCDH18, PDE2A, PDGFRA, PDGFRB, PDGFRL, PDLIM1, PDPN, PDZK1IP1, PERP, PI16, PI3, PKP1, PKP3, PLA2G4F, PLCH2, PLEC, PLEK2, PLEKHA1, PLIN3, PLP2, PLVAP, PLXDC1, PMFBP1, PPL, PPP1R13L, PPP1R14C, PPP2R3A, PPP4R1, PRG2, PROM2, PRRX1, PRRX2, PRSS22, PRSS27, PRSS3, PRSS8, PSAPLI, PTGES, PTGS1, PTPN21, PTPRF, PYDC1, RAB25, RAB3D, RAET1G, RAPGEFL1, RASAL1, RDH12, RHBG, RHCG, RHOD, RIMS3, RIN1, ROBO4, RORA, RPS6KA4, RSPO1, S100A14, S100A16, S100A2, S100A7, S100A8, S100A9, SBSN, SCNN1A, SDC1, SDCBP2, SDK1, SELP, SERPINB8, SFN, SFRP2, SFTPD, SGPP2, SH2D3A, SH3D19, SH3GL1, SIX2, SLC22A23, SLC24A3, SLC30A1, SLC47A2, SLC6A9, SLCO2A1, SLIT3, SLPI, SLURP1, SMAD1, SMAGP, SMPD3, SNAI2, SNX7, SORBS3, SOX15, SOX18, SOX7, SP6, SPARC, SPINT1, SPINT2, SPNS2, SPON1, SPRR1B, SPRR2D, SPRR2E, SPRR2F, SPRR4, SPTLC3, SSH3, ST14, STAB2, STEAP4, STMN2, STON2, SULT2B1, TACSTD2, TAX1BP3, TBX15, TFCP2L1, TGM1, TGM5, THBD, THRB, TMEM119, TMEM154, TMEM30B, TMEM45A, TMEM79, TMTC3, TNFAIP8L3, TNKS1BP1, TNXB, TP53AIP1, TP63, TPBG, TPPP3, TRIM7, TSHZ3, TSPAN11, TSPAN18, TSPO, TUBA4A, TUFT1, TWIST2, TYRP1, UNC5B, VASN, VDR, VGLL3, VSIG10L, WFDC12, WNT11, WNT3, WNT4, WNT5A, XG, ZBTB7C, ZC3H12A, ZNF185, ZNF296, ZNF385A, ZNF423, and ZNF521.

12. A method for treating melanoma in a patient having a tumor which comprises:
(a) determining or having determined if the tumor is positive or negative for a gene expression based biomarker, wherein the determination of whether the tumor is positive or negative is made by the method of claim 3; and
(b) administering to the patient a PD-1 antagonist if the tumor is positive for the gene expression based biomarker.

13. (canceled)

14. The method of claim 3, wherein the PD-1 antagonist is pembrolizumab.

15. (canceled)

16. (canceled)

17. (canceled)

18. (canceled)

19. (canceled)

20. (canceled)

21. A method of treating melanoma in a patient having a tumor which comprises:
(a) determining if the tumor has an elevated level of a gene expression based biomarker, wherein the determining step comprises:
a. obtaining a sample from the patient's tumor,
b. sending the tumor sample to a laboratory with a request to test the sample for the presence or absence of the gene expression based biomarker,
c. receiving a report from the laboratory that states whether the tumor sample is biomarker positive or biomarker negative, wherein the determination that the tumor sample is biomarker positive is made if the sample has elevated levels of gene expression of 5 or more genes from Table 1 and the determination that the tumor is biomarker negative is made if the sample has lower levels of expression of 5 or more genes from Table 1, and
(b) administering to the patient a PD-1 antagonist if the tumor is positive for the biomarker.

22. The method of claim 21, wherein the positive biomarker status is calculated through the expression of 5 or more up-regulated genes selected from the group comprising: A4GALT, ABLIM1, ADAM15, ADAM33, ADAMTS12, ADAMTS2, ADAMTS5, ADK, AGTR1, AHNAK, AHNAK2, AKR1C1, AKR1C2, AKR1C3, ALDH3A1, ALDH3B2, ALOXE3, ALS2CL, ANGPTL2, ANO1, ANPEP, ANXA2, ANXA9, APCDD1, APLNR, AQP1, AQP3, AQP5, ARHGEF15, ARHGEF19, ARHGEF4, ARL4D, ARNTL2, ASAP3, ASPN, ASPRV1, ATL3, ATP12A, ATP6V1C2, ATP8B1, B3GNT4, BDKRB2, BICC1, BICD2, BMP1, BMPR2, BOC, BSPRY, BTBD11, C12orf54, C19orf33, CA12, CALML3, CALML5, CAPN1, CAPNS2, CASZ1, CBLC, CCDC113, CCDC120, CCDC3, CCDC92, CCL22, CD109, CD24, CD248, CD34, CD44, CD9, CDA, CDH13, CDH3, CDHR1, CDR1, CDS1, CEACAM19, CH25H, CLDN1, CLDN4, CLEC14A, CLIC3, CLTB, CNFN, COL12A1, COL13A1, COL14A1, COL15A1, COL17A1, COL18A1, COL1A1, COL1A2, COL23A1, COL3A1, COL5A1, COL5A2, COL5A3, COL6A1, COL6A2, COL6A3, COL6A6, COL7A1, COL8A2, COMP, COMTD1, CPA3, CPA4, CPXM1, CPXM2, CPZ, CRABP1, CRABP2, CRCT1, CREB3L1, CRISPLD2, CRYM, CST6, CSTA, CTNNBIP1, CTSG, CTSK, CTTNBP2NL, CXADR, CXCL12, CXCL14, CYB561, CYB5R3, CYP26B1, CYP2S1, CYYR1, DAPL1, DAZAP2, DCN, DEGS1, DEGS2, DENND2C, DGAT2, DHRS1, DIO2, DMKN, DPP4, DPT, DSC2, DSEL, DSP, DST, DUOX1, DUOXA1, DUSP14, EBF1, ECSCR, EDN1, EFNA3, EFNB2, EGLN3, EHD2, ELMO3, ELOVL3, ELOVL4, ELOVL7, EML1, EMP1, EMP2, EN1, EPHA1, EPHB6, EPHX3, EPPK1, EPS8L1, ERBB2, ESRP2, ETS2, EVPL, EXPH5, F10, F2RL1, F2RL2, FADS6, FAM110C, FAM167A, FAM180A, FAM83F, FAM83H, FAT2, FAT4, FBLN1, FBLN2, FBN1, FCER1A, FGF11, FGFR3, FIBIN, FMO1, FOSL2, FOXQ1, FUT1, FZD10, GALNT1, GAN, GAS1, GDPD3, GJA1, GJB2, GJB3, GJB5, GJB6, GLT8D2, GLTP, GNA15, GNAL, GPC1, GPR68, GREM1, GRHL1, GRHL2, GSDMA, HAS3, HDC, HEBP2, HES2, HOPX, HOXD10, HR, HSD11B2, HSPA12B, HTRA1, ID1, IDE, IFF02, IGFBP4, IGFL2, IGFL4, IL1R1, IL1RN, IL20RB, IMPA2, IRX2, IRX3, IRX5, ISM1, ITGB4, IVL, JAM2, JMJD7-, LA2G4B, JUP, KCND3, KCNK6, KCNK7, KCTD11, KIAA1217, KIAA1522, KIF26A, KIT, KITLG, KLC3, KLF10, KLF11, KLF3, KLF4, KLF5, KLF6, KLK10, KLK5, KLK6, KLK8, KRT1, KRT10, KRT15, KRT17, KRT19, KRT2, KRT23, KRT31, KRT5, KRT78, KRT79, KRT80, KRTAP10-12, KRTDAP, LAD1, LAMA2, LAMA3, LAMB3, LCE1A, LCE1B, LCE1D, LCE1F, LCE2A, LCE3A, LCN2, LIMA1, LOXL1, LRRC15, LRRC32, LRRC8E, LTB4R, LTBP1, LUM, LY6D, LY6G6C, LYNX1, LYPD2, LYPD3, LYPD5, MAL2, MALL, MAP7, MARVELD1, MAST4, MEGF6, MEOX1, MFAP4, MFAP5, MICALL1, MINK1, MMP11, MMP2, MMP7, MMRN2, MN1, MPZL2, MRGPRF, MSX2, MXRA5, MXRA8, MYO6, NCCRP1, NDRG4, NDUFA4L2, NEURL1B, NFATC4, NGEF, NIPAL4, NKD2, NLRX1, NMU, NRARP, NTF3, NTN1, NUAK1, OLFM2, OLFML1, OLFML2A, OSR2, OTUB2, OVOL1, PAK6, PALLD, PALMD, PAPPA, PAQR7, PCDH18, PDE2A, PDGFRA, PDGFRB, PDGFRL, PDLIM1, PDPN, PDZK1IP1, PERP, PI16, PI3, PKP1, PKP3, PLA2G4F, PLCH2, PLEC, PLEK2, PLEKHA1, PLIN3, PLP2, PLVAP, PLXDC1, PMFBP1, PPL, PPP1R13L, PPP1R14C, PPP2R3A, PPP4R1, PRG2, PROM2, PRRX1, PRRX2, PRSS22, PRSS27, PRSS3, PRSS8, PSAPLI, PTGES, PTGS1, PTPN21, PTPRF, PYDC1, RAB25, RAB3D, RAETIG, RAPGEFL1, RASAL1, RDH12, RHBG, RHCG, RHOD, RIMS3, RIN1, ROBO4, RORA, RPS6KA4, RSPO1, S100A14, S100A16, S100A2, S100A7, S100A8, S100A9, SBSN, SCNN1A, SDC1, SDCBP2, SDK1, SELP, SERPINB8, SFN, SFRP2, SFTPD, SGPP2, SH2D3A, SH3D19, SH3GL1, SIX2, SLC22A23, SLC24A3, SLC30A1, SLC47A2, SLC6A9, SLCO2A1, SLIT3, SLPI, SLURP1, SMAD1, SMAGP, SMPD3, SNAI2, SNX7, SORBS3, SOX15, SOX18, SOX7, SP6, SPARC, SPINT1, SPINT2, SPNS2, SPON1, SPRR1B, SPRR2D, SPRR2E, SPRR2F, SPRR4, SPTLC3, SSH3, ST14, STAB2, STEAP4, STMN2, STON2, SULT2B1, TACSTD2, TAX1BP3, TBX15, TFCP2L1, TGM1, TGM5, THBD, THRB, TMEM119, TMEM154, TMEM30B, TMEM45A, TMEM79, TMTC3, TNFAIP8L3, TNKS1BP1, TNXB, TP53AIP1, TP63, TPBG, TPPP3, TRIM7, TSHZ3, TSPAN11, TSPAN18, TSPO, TUBA4A, TUFT1, TWIST2, TYRP1, UNC5B, VASN, VDR, VGLL3, VSIG10L, WFDC12, WNT11, WNT3, WNT4, WNT5A, XG, ZBTB7C, ZC3H12A, ZNF185, ZNF296, ZNF385A, ZNF423, and ZNF521.

23. (canceled)

24. (canceled)

25. A method of treating melanoma in a patient having a tumor which comprises:
(a) determining if the tumor has a lower expression level of a gene expression based biomarker, wherein the gene expression based biomarker comprises 5 or more genes from Table 2, wherein the determining step comprises:
(i) obtaining a sample from the patient's tumor,
(ii) sending the tumor sample to a laboratory with a request to test the sample for the presence or absence of the gene expression based biomarker,
(iii) receiving a report from the laboratory that states whether the tumor sample is biomarker positive or biomarker negative, wherein the determination that the tumor sample is biomarker positive is made if the sample has decreased levels of gene expression of 5 or more genes from Table 2 and the determination that the tumor is biomarker negative is made if the sample has elevated levels of expression of 5 or more genes from Table 2, and
(b) administering to the patient a PD-1 antagonist if the tumor is positive for the biomarker, wherein
(i) for the up-regulated gene expression signature, if the calculated signature score is equal to or greater than a pre-specified threshold, then the tumor is classified as biomarker positive, and if the calculated signature score is less than the pre-specified threshold, then the tumor is classified as biomarker negative, and
wherein the patient is determined to have a poor prognosis if the tumor is classified as biomarker positive or a favorable prognosis if the tumor is biomarker negative.

26. The method of claim 25, wherein the 5 or more up-regulated genes selected from the group comprising: ABHD10, ABHD3, ACVR2B, ADAL, ALG13, ANGEL1, ATG16L1, B4GALT3, BRAF, BRSK1, C12orf60, C1orf56, C4A, C7, CCDC151, CCDC93, CCNE1, CD1D, CD38, CD5L, CDC42SE1, CHEK2, CHORDC1, CMTM7, CPOX, CR1, CRELD1, CRNKL1, CSE1L, DARS2, DBNDD2, DDIT4, DEFB108B, DHODH, DNAJB9, DNAJC5B, DPM3, DTNB, EIF4A2, ERP29, ESM1, EXOC4, FAM122B, FANCL, FMNL2, FUBP1, GGA2, GHRH, GLUL, GPN3, HBE1, HELB, HEMK1, INPP5B, KCNJ10, L3MBTL1, LHFPL1, LIPT1, MAGED1, MBOAT1, MDM1, MERTK, METTL3, METTL7B, MGAT4A, MMD, MPI, MRM1, MSH6, MSI2, MSL2, NAPB, NBPF1, NDUFAF3, NLK, NT5DC3, OLIG2, OMA1, OXNAD1, P4HA1, PDIA4, PGBD2, PHF6, PIP5K1A, PMS2, POLR3K, PREPL, RAB3GAP2, RBM39, RBM45, RNF2, RRN3, SEC24A, SFXN2, SIGLEC11, SLC30A3, SNAPC3, SPAG4, SPIN3, SRPRB, SRSF9, STRBP, STX16, SYS1, TAF1A, TGM2, THOC2, TMEM182, TMEM81, TOP1, TP53BP1, TRIM5, TRNT1, TRPM2, UBFD1, URB2, VRK3, WDR76, WDSUB1, XPO1, ZMYND8, ZNF189, ZNF26, ZNF337, ZNF544, ZNF550, ZNF572, and ZNF841.

27. (canceled)

28. The method of claim 21, wherein the PD-1 antagonist is pembrolizumab.

29. (canceled)

30. (canceled)

31. (canceled)

32. (canceled)
